(19) United States
(12) Patent Application Publication
(10) Pub. No.: US 2004/0146516 A1
(43) Pub. Date: Jul. 29, 2004

(54) Lumen-exposed molecules and methods for targeted delivery

(75) Inventors: Paul Roben, San Diego, CA (US); Anthony C. Stevens, San Diego, CA (US)

Correspondence Address: WILSON SONSINI GOODRICH & ROSATI, 650 PAGE MILL ROAD, PALO ALTO, CA, 943041050

(73) Assignee: Utah Ventures II L.P., Salt Lake City, UT

(21) Appl. No.: 10/794,899

(22) Filed: Mar. 5, 2004

Related U.S. Application Data
(63) Continuation-in-part of application No. 10/165,603, filed on Jun. 7, 2002.
(63) Continuation-in-part of application No. 09/528,742, filed on Mar. 20, 2000.
(63) Continuation-in-part of application No. PCT/US03/10195, filed on Mar. 31, 2003.
(60) Provisional application No. 60/297,021, filed on Jun. 8, 2001.
(60) Provisional application No. 60/305,117, filed on Jul. 12, 2001.
(60) Provisional application No. 60/139,579, filed on Jun. 17, 1999.
(60) Provisional application No. 60/369,452, filed on Apr. 1, 2002.

(51) Int. Cl. A61K 48/00; A61K 39/395

(52) U.S. Cl. 424/178.1; 514/44

(57) ABSTRACT

The present invention provides novel methods and kits for labeling and isolating tissue-specific or organ-specific lumen-exposed molecules. In addition, the present invention provides tissue-specific or organ-specific lumen-exposed polypeptides, which were isolated by the methods herein. Furthermore the present invention provides therapeutic complexes comprising ligands that bind the tissue-specific or organ-specific lumen-exposed polypeptides attached to therapeutic moieties for targeted treatment and prevention.

### CROSS REFERENCE

This application is a continuation-in-part of U.S. patent application Ser. No. 10/165,603, filed Jun. 7, 2002, which claims priority to U.S. Provisional Application Serial No. 60/297,021, filed Jun. 8, 2001 and 60/305,117, filed Jul. 12, 2001. This application is also a continuation-in-part of U.S. patent Ser. No. 09/528,742, filed Mar. 20, 2000, which claims priority to U.S. Provisional Application Serial No. 60/139,579, filed Jun. 15, 1999. This application is also a continuation-in-part of PCT/US03/10195, filed Mar. 23, 2003, which claims priority to U.S. Provisional Application Serial No. 60/369,452, filed Apr. 1, 2003. All of the above references are incorporated herein by reference in their entirety for all purposes

### BACKGROUND

Currently, when drugs are conventionally administered to a patient, they circulate throughout the entire body of the patient. As a result, exntremely high dosages are required to reach therapeutic levels in the desired organ. This non-targeted delivery of high dosages of drugs results in systemic toxicity and severe side-effects.

Targeted delivery of therapeutic or diagnostic agents to specific organs or tissues is much safer and more effective than delivery of a drug to an entire individual, as is the case by conventional administration techniques. The ability to specifically deliver a composition (e.g., a drug or gene) to a specific organ or tissue in vivo allows much smaller amounts of the drug to be administered thereby reducing associated side effects.

Conventional means to achieve this sort of “targeted” or organ-specific delivery includes the use of implants (e.g., Elisseeff (1999) Proc. Natl. Acad. Sci. USA 96:3104-3107), stents or catheters (see, e.g., Murphy (1992) Circulation 86:1596-1604), or vascular isolation of an organ (e.g., liver, see, e.g., Vahrmeijer (1998) Semin. Surg. Oncol. 14:262-268). However, these techniques are invasive, traumatic and can cause extensive inflammatory responses and fibrocellular proliferation (see, e.g., van der Giessen (1996) Circulation 94:1690-1697).

A more sophisticated strategy is the targeted delivery of compounds to a tissue-specific or organ-specific molecule exposed on the luminal surface of the vasculature. Previous attempts at tissue-specific or organ-specific delivery depended on sites within the tissue that were inaccessible to the compounds due to the natural barrier of the vasculature. Hence the importance of identifying accessible, tissue-specific or organ-specific molecules exposed on the luminal surface of the vasculature. For example, vasculature-targeted chemotherapy, i.e., the destruction of tumor blood vessels with cytotoxic agents, makes use of biochemical differences between angiogenic and resting blood vessels (see, e.g., Ruoslahti (1999) Adv. Cancer Res. 76:1-20). This approach may minimize or eliminate some of the problems associated with conventional solid-tumor targeting, such as poor tissue penetration and drug resistance. Eliminating tumor blood supply using anti-angiogenic agents can have dramatic anti-tumor effects. Targeting chemotherapeutic agents to the tumor vasculature kills tumor blood vessels in addition to having the usual anti-tumor activities of the drug. This approach can result in increased efficacy and reduced toxicity of anti-tumor agents.

However, the versatility and scope of any biochemical targeting strategy is dependent on the in vivo or in situ identification of tissue-specific or organ-specific molecules expressed on the luminal surface of the vasculature. One strategy is to identify tissue-specific or organ-specific molecule differences in vivo is by screening peptide libraries expressed on the surface of bacteriophage (see, e.g., Rajotte (1998) J. Clin. Invest. 102:430-437). However, this method may miss many potential tissue-specific or organ-specific molecules because it is dependent on the ability of fusion proteins to bind to cell surface molecules with sufficient affinity to isolate such molecules.

Another strategy is to selectively radioiodinate lumen-exposed polypeptides in situ. (see, e.g., Schnitzer (1990) Eur. J. Cell Biol. 52:241-251). However, this method is limited because it only labels polypeptides containing tyrosine residues and does not facilitate isolating the labeled molecule.

Another approach coats lumen-exposed cells with cationized silica particles followed by polyanion crosslinkers in situ. (See, e.g., Schnitzer, et al., U.S. Pat. Nos. 5,281,700; 5,776,770; 5,914,127). Tissue is then homogenized and cell membranes bound to the silica are isolated by density gradients. This method may result in a significant fraction of non-lumen-exposed molecules contaminating the isolated fraction. In the Schnitzer-silica particle technique, once the cells are homogenized, all intracellular molecules can bind to the silica-polyanion complex. When whole membranes are isolated with this technique, molecules not exposed to the luminal surface are also isolated.

Another approach used in situ was to label isolated lung proteins by perfusing the pulmonary artery with the non-cleavable cell membrane impermeant biotinylation reagent sulfosuccinimidyl 6-biotin-amido hexanoate, which labels amine groups of polypeptides (De La Fuente (1997) Amer. J. of Physiol. 272:L461-L470). Tissue homogenates were incubated with streptavidin-agarose beads. Elution of the biotinylated polypeptides from the streptavidin required harsh denaturing conditions, as the biotin-stepavidin binding affinity is approximately 10⁻¹⁵ M⁻¹. This resulted in significant contamination with non-specifically binding polypeptides and other non-lumen exposed molecules in the eluate. This method is also flawed in that significant amounts of naturally biotinylated proteins not normally exposed to the lumen in vivo are also isolated.

Because of the increased demand for use of more sophisticated drug delivery techniques, such as the biochemical strategy of targeted delivery of drugs and genes to only specific organs and/or tissues, different ways of identifying and isolating tissue-specific or organ-specific molecules are needed. The present invention addresses these and other needs.

### SUMMARY OF THE INVENTION

This invention provides novel methods and kits to label and isolate lumen-exposed molecules, particularly polypeptides, that are expressed in a tissue-specific or organ-specific manner on the luminal side of cells lining perfusible spaces. This invention also provides compositions and methods for targeting specific tissues and delivering therapeutics to such tissues.

In one aspect, the present invention provides a method of labeling a molecule exposed on a luminal surface of a perfusible space in situ or in vivo comprising the steps of providing a cell membrane impermeable reagent comprising three domain: (i) a first domain comprising a chemical moiety capable of covalently and non-specifically binding to a molecule exposed on the luminal surface of a cell lining a perfusible space in situ and in vivo; (ii) a second domain comprising a labeling domain; and (iii) a third domain situated between the first and second domains linking the first domain and the second domain by a cleavable chemical moiety. The cleavable chemical moiety preferably does not cleave under in vivo conditions but will cleave under reducing but not denaturing conditons. To label a tissue-specific or organ-specific molecule, the membrane impermeable reagent is administered into the perfusible space of an intact organ or an intact animal to react the cell membrane impermeable reagent with the molecules expressed on the luminal surface of the cell lining of the perfusible space and label a lumen-exposed molecule.

In alternative embodiments, the present invention provides a method of isolating tissue-specific or organ-specific molecules by adminstering in vivo, in situ, or in vitro, into a perfusible space a cell membrane impermeable reagent wherein the second domain comprises a binding domain. Furthermore, a ligand may be added wherein the ligand binds to the binding domain of the cell membrane impermeable reagent.

In another aspect, the present invention provides tissue-specific or organ-specific therapeutic complexes wherein the therapeutic complex comprises (i) a ligand which binds to a tissue-specific or organ-specific lumen-exposed molecule; (ii) a therapeutic moiety; and (iii) a linker that links the ligand to the therapeutic moiety. Such therapeutic complexes can be used to diagnose conditions associated with expression, over-expression, or under-expression of lumen-exposed molecules and treat conditions that would benefit from targeted therapeutics.

The details of the invention are set forth in the accompanying drawings and the description below. A further understanding of the nature and advantages of the present invention is realized by reference to the remaining portions of the specification, the figures and claims.

All publications, patents and patent applications cited herein are hereby expressly incorporated by reference for all purposes.

### BRIEF DESCRIPTION OF THE DRAWINGS

FIG. 1 is a depiction of a typical therapeutic complex interacting with an endothelial cell surface, tissue-specific molecule.

FIGS. 2A-D show the immunohistochemistry of tissue sections from a rat which was injected with either CD71 or a control antibody. FIG. 2A is brain from a rat injected with CD71, FIG. 2B is brain from a rat injected with the control antibody, FIG. 2C is lung from a rat injected with CD71, FIG. 2D is lung from a rat injected with the control antibody.

FIG. 3 shows a polyacrylamide gel of luminal proteins isolated from lung. Dipeptidyl peptidase IV is labeled DPP-4.

FIGS. 4A-F are a series of immunohistograms of various tissues showing binding of an anti-dipeptidyl peptidase antibody to luminal tissue in kidney and lung.

FIG. 5 shows a polyacrylamide gel of another set of luminal proteins isolated from lung. Carbonic Anhydrase IV is labeled CA-4.

FIG. 6 shows a polyacrylamide gel of luminal proteins isolated from pancreas. Zymogen granule 16 protein is labeled ZG16P.

FIGS. 7A-F are a series of immunohistograms of various tissues showing binding of a MAdCAM antibody to luminal tissue in pancreas and colon.

FIGS. 8A-F are a series of immunohistograms of various tissues showing binding of a Thy-1 (CD90) antibody to luminal tissue in the kidney.

FIG. 9 shows a polyacrylamide gel of luminal proteins isolated from prostate. The albumin fragment is labeled T406-608.

FIGS. 10A-D are a series of immunohistograms of various tissues showing binding of OX-61 to dipeptidyl peptidase N, which is expressed on the luminal surface of the vasculature of the lung.

FIGS. 11A-D are a series of immunohistograms of various tissues showing binding of OST-2 to MadCam-1, which is expressed on the luminal surface of the vasculature of the pancreas and colon.

FIGS. 12A-F are a series of immunohistograms of various tissues showing binding of OX-7 to CD90, which is expressed on the luminal surface of the vasculature of the kidney.

FIGS. 13A-F are a series of immunohistograms of various tissues showing binding of an anti-carbonic anhydrase IV antibody to carbonic anhydrase IV, which is expressed on the luminal surface of the vasculature of the heart and lung.

FIGS. 14A-E are a series of immunohistograms of lung showing a profile of the binding of OX-61 to dipeptidyl peptidase IV over a twenty-four hour timecourse.

FIGS. 15A-D are a series of immunohistograms of pancreas showing a profile of the binding of OST-2 to MadCam-1 over a forty-eight hour timecourse.

FIGS. 16A-F are a series of immunohistograms of kidney showing a profile of the binding of OX-7 to CD90 over an eight hour timecourse.

FIGS. 17A-C are graphs which show the fraction of the injected dose of Europium-labeled OX-61 that localized to lung over a twenty-four hour time period. The dashed line indicates the maximum level of isotype control antibody that bound to any of the indicated tissues at any time point.

FIGS. 18A-C are graphs which show the fraction of the injected dose of Europium-labeled anti-influenza IgG2A isotype control antibody that localized to specific tissues over a twenty-four hour time period.

FIGS. 19A-C are graphs which show the fraction of the injected dose of Europium-labeled OST-2 that localized to pancreas over a twenty-four hour time period. The dashed line indicates the maximum level of isotype control antibody that bound to any of the indicated tissues at any time point.

FIG. 20 is a graph which shows the fraction of the injected dose of Europium-labeled anti-carbonic anhydrase IV antibody that localized to heart and lung over a twenty-four hour time period.

FIG. 21 is a graph which shows the amount of injected ¹²⁵I-labeled OX-61 that localized to various tissues and fluids over an eight hour time period.

FIG. 22 is an immunohistogram of a section of lung which shows the transcytotic transport of OX-61 by dipeptidyl peptidase IV.

FIG. 23 is an immunohistogram of a section of kidney which shows the transcytotic transport of OX-7 by CD90.

FIG. 24 is an immunohistogram of a section of pancreas which shows that OST-2 binds to MadCam-1 on the luminal surface of the vasculature but is riot transported across the endothelium.

FIG. 25 is an immunohistogram of a section of lung which shows that anti-carbonic anhydrase IV antibody binds to carbonic anhydrase IV on the luminal surface of the vasculature but is not transported across the endothelium.

FIGS. 26A-F are a series of immunohistograms of various tissues showing binding of an OX-61/gentamicin therapeutic complex to dipeptidyl peptidase IV, which is expressed on the luminal surface of the vasculature of the lung.

FIGS. 27A-D are a series of immunohistograms of various tissues showing binding of an OX-61/doxorubicin therapeutic complex to dipeptidyl peptidase IV, which is expressed on the luminal surface of the vasculature of the lung.

FIG. 28 is an immunohistogram of a section of lung which shows the transcytotic transport of an OX-61/gentamicin therapeutic complex by dipeptidyl peptidase IV.

FIG. 29 is an immunohistogram of a section of lung which shows the transcytotic transport of an OX-61/doxorubicin therapeutic complex by dipeptidyl peptidase IV.

FIGS. 30A-F are a series of immunohistograms of various tissues showing binding of an OST-2/gentamicin therapeutic complex to MadCam-1, which is expressed on the luminal surface of the vasculature of the colon and pancreas.

FIGS. 31A-F are a series of immunohistograms of various tissues showing binding of an OST-2/doxorubicin therapeutic complex to MadCam-1, which is expressed on the luminal surface of the vasculature of the colon and pancreas.

FIGS. 32A-B are graphs which show the amount of free gentamicin that accumulated in the lung and the kidney over an eighteen hour time period compared to the amount that was delivered to these tissue in DSPC-DPP therapeutic complexes.

FIGS. 33A-B are graphs which show the amount of free gentamicin that accumulated in various tissues over an eighteen hour time period compared to the amount that was delivered to these tissue in EPC-DPP therapeutic complexes and untargeted liposomes.

FIGS. 34A-B are graphs which show the amount of free gentamicin that accumulated in various tissues over an eighteen hour time period compared to the amount that was delivered to these tissue in DSPC-DPP therapeutic complexes and untargeted liposomes.

FIG. 35 is a graph which shows the efficacy of both free gentamicin and gentamicin in EPC-DPP therapeutic complexes in the treatment of lung infections.

FIG. 36 depicts a photograph of an SDS polyacrylamide gel that shows an approximately 40 kDa polypeptide that is present in the sample of pig brain but which is not present in the other tissues.

FIG. 37 depicts a photograph of an SDS polyacrylamide gel that shows an approximately 85 kDa polypeptide that is present in the sample of pig brain but which is not present in the other tissues.

FIG. 38 depicts a photograph of an SDS polyacrylamide gel that shows an approximately 35 kDa polypeptide that is present in the sample of pig brain but which is not present in the other tissues.

FIG. 39 depicts a photograph of an SDS polyacrylamide gel that shows an approximately 80 kDa polypeptide that is present in the sample of pig heart but which is not present in the other tissues.

FIG. 40 depicts a photograph of an SDS polyacrylamide gel that shows two approximately 47 kDa polypeptides that are present in the sample of pig heart but which is not present in the other tissues.

FIGS. 41A-C depict a photograph of SDS polyacrylamide gels that shows an approximately 55 kDa polypeptide that is present in the sample of pig heart but which is not present in the other tissues.

FIG. 42 depicts a photograph of an SDS polyacrylamide gel that shows an approximately 17 kDa polypeptide that is present in the sample of pig heart but which is not present in the other tissues.

FIG. 43 depicts a photograph of an SDS polyacrylamide gel that shows an approximately 125 kDa polypeptide that is present in the sample of pig heart but which is not present in the other tissues.

FIG. 44 depicts a photograph of an SDS polyacrylamide gel that shows an approximately 100 kDa polypeptide that is present in the sample of pig lung and heart but which is not present in the other tissues.

FIG. 45 depicts a photograph of an SDS polyacrylamide gel that shows an approximately 25 kDa polypeptide that is present in the sample of pig lung but which is not present in the other tissues.

FIGS. 46A-D depict photographs of two-dimensional gels that show an approximately 48 kDa polypeptide that is present in the sample of lung but which is not present in the other tissues.

FIGS. 47A-D depict photographs of two-dimensional gels that show an approximately 125 kDa polypeptide that is present in the sample of lung but which is not present in the other tissues.

FIGS. 48A-D depict photographs of two-dimensional gels that show an approximately 45 kDa polypeptide that is present in the sample of pig pancreas but which is not present in the other tissues.

FIGS. 49A-D show the immunohistochemistry of tissue sections from a rat which was injected with either an antibody specific for CD71 (OX-26) or a control (albumnin specific) antibody. FIG. 49A shows brain from a rat injected with biotin-labeled OX-26, FIG. 49B shows brain from a rat injected with biotin-labeled monoclonal antibody specific for albumin, FIG. 49C shows lung from a rat injected with biotin-labeled OX-26, FIG. 49D shows lung from a rat injected with biotin-labeled monoclonal antibody specific for albumin.

FIGS. 50A-E are a series of immunohistograms showing various tissue sections taken from a rat that was injected with a biotin-labeled monoclonal antibody specific for folate binding protein.

FIGS. 51A-F are a series of immunohistograms showing various tissue sections taken from a rat that was injected with gentmicin that was linked to a monoclonal antibody specific for folate binding protein.

FIG. 52 illustrates a representation of a stained polyacrylamide gel electrophoresis (PAGE) separating lumen-exposed molecules from rat brains, lungs, kidneys, hearts, liver and fat, isolated using an exemplary method of the invention, as described in Example 1, below.

FIG. 53 illustrates a representation of a stained PAGE separating polypeptides eluted from beads under both “mild conditions” (left panel) (i.e., an exemplary method of the invention) and “harsh conditions” (right panel); harsh conditions being boiling in a sample buffer as described in Example 39, below.

FIG. 54 (upper panel) illustrates a representation of the results of a Western blot of a PAGE separating vascular lumen-exposed polypeptides, prepared by the methods of the invention, stained with an antibody that recognizes a polypeptide that is only expressed on the lumen of vascular endothelial cells (PECAM-1) and an antibody that recognizes a polypeptide only expressed intracellularly (the Golgi 58 kDa polypeptide), as described in Example 39, below. FIG. 54A (lower panel) shows a Western blot of total tissue homogenate stained with anti-Golgi 58 kDa polypeptide antibody.

FIG. 55 illustrates a representation of the results of a protein stained PAGE (FIG. 55A) and a Western blot of this gel probed with a streptavidin-fluorescent probe (FIG. 55B), as described in detail in Example 40. Streptavidin beads added to membrane preparation to purify naturally biotinylated proteins were first eluted using “milder” elution conditions (left lanes of FIGS. 55A and 55B); followed by elution under “harsh conditions” (right lanes of FIGS. 55A and 55B), as described in Example 40.

FIG. 56 illustrates a representation of the results of a protein stained PAGE comparing harsh elution conditions with LC-Biotin versus mild elution conditions with S-S biotin for the elution of proteins from liver and heart preparations, as described in Example 2.

FIG. 57 illustrates a representation of a PVDF of a “Grid Digest” identifying organ-specific luminal-exposed vascular proteins wherein B=brain, C=colon, H=heart, K=kidney, Li=liver, Lu=lung, Pa=panaceas, Smin=small intestine.

FIG. 58 illustrates a representation of a PVDF used for N-terminal sequencing identification of an organ-specific luminal exposed vascular protein.

### DETAILED DESCRIPTION OF THE INVENTION

In one aspect, the present invention provides a novel means to label and/or isolate molecules, particularly polypeptides, which are exposed on the lumen side of cells lining perfusible spaces in a tissue, organ or whole intact organism. These perfusible spaces include, e.g., vascular, ductal, CSF space, peritoneum, eye, fascial spaces, and other perfusible tissue spaces. In particular the tissue-specific or organ-specific lumen-exposed molecules identified are well suited for “tagging” the particlular tissue or organ from which they are derived. The “tagged” reagent-reacted molecule can be reacted with a binding domain ligand (e.g., avidin, where the binding domain is biotin) for isolation. In particular, a reagent-reacted or “tagged” lumen-exposed molecule may be isolated by washing away of non-reagent reacted molecules (e.g., substantially all non-bound molecules), followed by cleaving of the chemical moiety under conditions whereby none or an insignificant amount of binding domain is separated from its ligand. Other embodiments for the methods of identifying tissue-specific and organ-specific molecules are identified in U.S. patent applciation Ser. No. 09/528,742, filed Mar. 20, 2000, incorporated herein by refrence for all purposes.

The method disclosed in U.S. patent application Ser. No. 09/528,742 permits the in vivo isolation of all proteins that are exposed on the inner surface of blood vessels from different tissues. All other proteins that make up the tissues (which are the vast majority) are discarded in the process. The resulting set of luminally exposed vascular proteins can then be separated and analyzed biochemically to identify each protein individually. By comparing the set of proteins expressed in each tissue, proteins are identified that are specific to a given tissue. Proteins of interest are then sequenced. Ligands are obtained that specifically bind to the target protein. These ligands, upon binding to the target protein, or the protein that is tissue-specifically luminally expressed, preferably does not activate a specific signal transduction pathway in the cell it binds to, but may activate the process of transcytosis or pinocytosis.

In another aspect, the present invention provides both compositions and methods for delivery to a specific tissue or organ whether or not such a tissue or an organ is in a diseased state. Specifically, the invention utilizes tissue-specific or organ-specific lumen-exposed molecules to localize the therapeutic complexes described herein by binding these complexes to the lumen-exposed molecules. This embodiment allows for localization and concentration of a pharmaceutical agent to a specific tissue or organ, thus increasing the therapeutic index of the pharmaceutical agent. Localization also decreases the chances of side effects and may allow one to use a lower concentration of the agent to achieve the same results. Accordingly, the agents that may have previously been considered effective but with unacceptable side effects may be rendered usable. Localization to a lumen-exposed tissue-specific or organ-specific molecules affords the added advantage that a single ligand can be used to treat a variety of diseases involving the same tissue or organ. For example, a tissue-specific or organ-specific ligand can be used to target different therapeutic agents depending on the disease state of the tissue in need.

I. Definitions

The term “avidin” as used herein means any biotin-binding compound such as avidin, streptavidin, any modified or mutant avidin produced by laboratory techniques which is capable of binding biotin or a functional equivalent of biotin, any compound designed to function like avidin, and equivalents thereof. See, e.g., Green (1970) Methods Enzymol. 18A:418-424; Green (1965) Biochem. J. 94:23c-24c; Schray (1988) Anal. Chem. 60:853-855; Mock (1985) Analytical Biochem. 151:178-181; Ding (1999) Bioconjug. Chem. 10:395-400; U.S. Pat. No. 6,022,951.

The term “biotin” as used herein means biotin, any modified biotin, and also includes biotin analogs and equivalents thereof, e.g., biotin methyl ester, desthiobiotin, diaminobiotin or 2-iminobiotin. See, e.g., Hofmann (1982) Biochemistry 21:978-984; Reznik (1998) Proc. Natl. Acad. Sci. USA 95:13525-13530; Torreggiani (1998) Biospectroscopy 4:197-208.

The term “cell membrane impermeable reagent” as used herein means a reagent that cannot enter or pass through the lipid bilayer of a cell membrane; e.g., the cell membrane impermeable reagents of the invention, when perfused into tissue spaces, will only bind to molecules exposed to the lumen of the space (assuming the membranes of the cells lining the lumen are intact).

The term “homolog” or “homologous” as used herein refers to a polypeptide or an oligonucleotide having at least 50%, more preferably 60%, more preferably 70%, more preferably 80%, or more preferably 90% identitical or similar monomer units as compared to a selected amino acid or nucleic acid sequence; or to a polypeptide or an oligonucleotide having at least 5, more preferably 10, more preferably 20, more preferably 40, or more preferably 80 consecutive monomer units (e.g., amino acid, nucleic acid, peptide nucleic acid) that are identical or similar to a selected amino acid or nucleic acid sequence; or to a portion, modification or derivative of a selected amino acid or nucleic acid sequence; or to a polypeptide or oligonucleotide that is functionally identical or similar to a selected amino acid or nucleic acid sequence (e.g., same gene or protein only from a different animal). Identity or similarity of nucleic acids may be determined using the FASTA version 3.008 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997)).

The term “intact organ” as used herein means an organ, or a section or piece thereof, whose basic anatomical architecture is intact, e.g., its vasculature (e.g., venules, arterioles, capillaries, lymph) or sinus spaces or the like have not been disrupted such that perfusion of a cell membrane impermeable reagent into the lumen of the vessel or sinus (or other perfusible space) will only label lumen-exposed molecules.

The term “isolated,” as used herein, when referring to a molecule or composition (e.g., an isolated cell-membrane impermeable reagent or tissue- or organ-specific molecule) means that the molecule or composition is separated from at least one other compound, such as a protein, DNA, RNA, lipid, carbohydrate, or other contaminants with which it is associated in vivo or in its naturally occurring state. Thus, a tissue- or organ-specific molecule is considered isolated when it has been isolated from any other component with which it is naturally associated. An isolated composition can, however, also be substantially pure. An isolated composition can be in a homogeneous state. It can be in a dry or an aqueous solution. Purity and homogeneity can be determined, e.g., using any analytical chemistry technique, as described herein.

The term “luminal surface” or “lumen” as used herein means the surface of any perfusible space, e.g., the lumen-exposed surface of cells lining a perfusible space, e.g., endothelial cells in a vascular space (e.g., the lumen of an artery, vein, capillary, sinus, and the like).

The term “organ-specific molecule” or “tissue specific molecule” as used herein refers to a molecule (e.g., polypeptide, lipid, carbohydrate, etc.) that is preferentially expressed on a specific tissue (e.g., muscles, skin), organ (e.g., liver, lung, heart, brain), group of organs (e.g., all nervous or digestive tract tissues or organs) or cell type (e.g., hematopoietic cells), allowing a majority of the therapeutic complex to bind to that tissue, organ, group of organs or cell types after administration. Tissue-specific or organ-specific molecules can also include those expressed on normal versus pathological sets of cells (e.g., as with tumor specific antigens); those expressed on developmentally distinct phenotypes (e.g., polypeptides in angiogenic blood vessels versus those in “resting”/non-growing blood vessels). Tissue-specific or organ-specific molecules may be found at a considerably higher concentration in one or a few tissues than in the others. For example, a tissue-specific or organ-specific molecule may be highly upregulated in the lung compared to other tissues but can be dosed to be even more specific based on the statistical distribution of binding throughout the vasculature. Proper, often lower, dosing of the therapeutic complex would be given such that the amounts that appear randomly at non-targeted tissue would render little or no side effects.

The term “full-length” as used herein when referring to a polynucleotide means a polynucleotide sequence that comprises an entire polypeptide coding region that is flanked by at least one start codon and at least one stop codon and encodes a full-length polypeptide. When referring to a polypeptide “full-length” means a protein having the amino acid sequence of a protein that is functional when expressed in its native state in vivo or an unprocessed precursor thereof. Although the sequence of the full-length polypeptide may correspond to the sequence of the functional protein, the full-length polypeptide need not be functional.

The terms “peptide,” “protein” and “polypeptide” as used herein are interchangeable. Additionally, the terms “lumen exposed” and “luminally expressed” are used interchangeably.

The term “perfusible space” as used herein means any tissue or organ space that can be perfused with a cell-impermeant reagent, e.g., any vascular or lymphatic lumen, the CSF space, lumens of ducts, vitreous-aqueous humor space of the eye, fascial planes, and the like, including spaces only present under disease, inflammatory or other conditions, e.g., cysts, tumors, and the like.

The term “target protein” as used herein means a tissue-specific or organ-specific lumen-exposed protein.

The term “ligand” as used herein means a molecule that specifically binds to or has affinity to a tissue-specific or organ-specific molecule. The amount of affinity necessary to be “specifically bound” can be determined functionally.

The term “linker” as used in conjunction with a therapeutic complex refers to any bond, molecule or other vehicle that links or connects the ligand and the therapeutic moiety.

The term “therapeutic moiety” as used herein refers to any type of substance, which can be used to affect a certain outcome. The outcome can be positive or negative. Alternatively, the outcome can simply be diagnostic. The outcome may also be subtler such as simply changing the molecular expression in a cell. The therapeutic moiety may also be an enzyme, which allows conversion of a prodrug into the corresponding pharmaceutical agent. Examples of therapeutic moieties include, but are not limited to, antibodies, antiviral agents, antifungal agents, antisense molecules, radionucleotides, proteins, a small or large organic or inorganic molecule, polysaccharides, immunomodulators, immunosuppressors, chemotherapeutic agents, antineoplastic agents, contrast agents, prodrugs, hormonal agents, and toxins. Examples of immunomodulators, include, but are not limited to, azathioprine, 6-mercaptopurine, cyclosporine, methotrexate, interleukin-2, beta-D-glucan and beta-D-glucan protein complex, OX-2, interleukin-10/mda-7, poxvirus growth factor, serpins, and a type I interferon-binding protein.

II. Methods of Detection and Isolation

The methods herein can be practiced in conjunction with any method or protocol known in the art and described in the scientific and patent literature. The various compositions (e.g., natural or synthetic compounds, polypeptides, peptides, nucleic acids, and the like) used to practice the methods herein can be isolated from a variety of sources, genetically engineered, amplified, and/or expressed recombinantly. Alternatively, these compositions (e.g., any or all domains of the membrane impermeable reagents of the invention) can be synthesized in vitro by well-known chemical synthesis techniques, as described in, e.g., Organic Syntheses Collective Volumes, Gilman et al. (Eds) John Wiley & Sons, Inc., NY; Venuti (1989) Pharm Res. 6:867-873; Carruthers (1982) Cold Spring Harbor Symp. Quant. Biol. 47:411-418; Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Beaucage (1981) Tetra. Lett. 22:1859; U.S. Pat. No. 4,458,066.

Techniques for the manipulation and isolation of organs, tissues, cells, nucleic acids, polypeptides are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Cell Membrane Impermeant Reagents

The invention provides methods for labeling and isolating molecules exposed to perfusible spaces, particularly tissue-specific and organ-specific molecules. Alternatively, the methods can be used to identify and isolate molecules expressed only under certain conditions, e.g., at particular stages of development or aging (e.g., “senescent endothelial cells,” see, e.g., Garlanda (1997) Arterioscler. Thromb. Vasc. Biol. 17:1193-1202), after exposure to particular hormones or cytokines (e.g., lymphokines), in inflamed, infected, or diseased tissues, molecules preferentially expressed on one or more tissues, or the like.

The cell membrane impermeable reagents have at least three domains: a first domain comprising a chemical moiety capable of covalently and non-specifically binding to a molecule expressed on the luminal surface of a cell lining a perfusible space in situ or in vivo; a second domain having a labeling domain (for labeling) or a binding domain (for isolating); and, a third domain situated between the first and second domains linking the first domain to the second domain by a cleavable chemical moiety, wherein the cleavable chemical moiety will not cleave under in vivo or physiologic (or equivalent) conditions and can be cleaved under relatively mild conditions.

The cell membrane impermeable reagents can be administered, for example, in vivo or in situ, into a perfusible space whereupon the first domain binds covalently and non-specifically to molecules expressed on the luminal surface of tissues. The second domain (e.g., labeling domain or binding domain) can then be utilized to detect and/or isolate the lumen-exposed molecules. By comparing molecules exposed on various luminal surfaces, the identification of molecules unique to a particular tissue(s) can be identified.

In any of the embodiments herein, the cell membrane impermeable reagent can further comprise a fourth domain that is either a labeling domain or a binding domain.

Moieties Capable of Covalent and Non-Specific Binding to Luminal Molecules

The first domain of the cell membrane impermeable reagent comprises a chemical moiety capable of covalently and non-specifically binding to a molecule expressed on the luminal surface of a cell lining a perfusible space in situ or in vivo. The moiety can be reactive to, e.g., amine, carboxyl, carbohydrate or sulfhydryl groups on the luminally-expressed molecule. The chemistry and, reagents for such reactions are well known in the art; see, e.g., catalog of Pierce Chemicals (Rockville, Ill.); http://www.piercenet.com/Products/.

Chemical moieties capable of covalently and non-specifically binding lumen-exposed molecules include amine reactive moieties, e.g., sulfo-NHS ester groups. They react to form a stable covalent bond with amine groups at a pH of about 7 to 9. Such exemplary membrane impermeable cross-linking reagents (which are cleavable) include: thiobis-(sulfosuccinimidyl) proprionate groups or sulfosuccinimidyl suberate (see, e.g., Conrad (1985) Int. Arch. Allergy Appl. Immunol. 77:228-231); sulfosuccinimidyl-2-(biotinamido) ethyl-1,3-dithiopropioate, e.g., Sulfobiotin-X-NHS™, (Pierce Chemicals catalog, 21331T). These compounds can be designed to be cleavable under mild, reducing conditions, using, e.g., dithiothreitol (DTT); mild conditions being preferably 8-50 mg/ml DTT, pH 6-12, 20-25° C. for about 1-30 minutes, more preferably 9-25 mg/ml DTT, pH 7-11, 21-24° C. for about 1-15 minutes, or even more preferably 10-15 mg/ml DTT, pH 8-10, 22-23° C. for about 1-5 minutes. See also, e.g., Shimkus (1985) Proc. Natl. Acad. Sci. 82:2593-2597; Duhamel (1985) J. Histochem. Cytochem. 33:711-714; Gottardi (1995) Am. J. Physiol. 268: F285-F295; Soukup (1995) Bioconjugate Chemistry 6: 135-138.

Other useful chemical moieties capable of covalently and non-specifically binding lumen-exposed molecules are consumable catalysts, e.g., crosslinking agents such as carbodiimide or carbamoylonium (see, e.g., U.S. Pat. Nos. 4,421,847; 4,877,724). With these crosslinking agents, one of the reactants must have a carboxyl group and the other an amine or sulfhydryl group. The crosslinking agent first reacts selectively with the carboxyl group, preferably a carboxyl group on a protein, then is split out during reaction of the “activated” carboxyl group with an amine on the crosslinking reagent, to form an amide linkage between the protein and crosslinking agent, thus covalently bonding the two moieties. See, e.g., U.S. Pat. No. 5,817,774.

Alternatively, sulfhydryl reactive moieties can be used, e.g., maleimide reactive groups such as N-(4-carboxycyclohexylmethyl)maleimide groups can acylate in aqueous or organic media within 2 minutes at room temperature. Maleimide reacts with —SH groups at pH 6.5 to 7.5, forming stable thioether linkages. See, e.g., U.S. Pat. Nos. 5,063,109 and 5,053,520.

Carbohydrate-binding moieties an also be used, e.g., an oxidized carbohydrate specific hydrazide, such as 4-(4-N-Maleimidophenyl) butyric acid hydrazide hydrochloride and its homologues having 2 to 8 carbon atoms in the aliphatic chain connecting the carbonyl and phenyl groups of the spacer. See, e.g., U.S. Pat. Nos. 6,015,556; 5,889,155.

Binding Domains

In various embodiments, the second domain of the cell membrane impermeable reagent comprises a binding domain. The term “binding domain” refers to any molecular entity that has a binding affinity to a second molecular entity referred to herein as a ligand. Binding domains are useful in detection, purification and isolation of tissue-specific or organ-specific molecules.

In one embodiment, the binding domain can be any chemical moiety having a known ligand that can be manipulated to identify the lumen-exposed molecule or to isolate such molecule. Preferably, a binding domain moiety has substantially little affinity for most naturally occurring molecules, or in particular, those that would otherwise be expected to be present in a tissue assayed. Alternatively, if the binding domain moiety has a significant affinity for certain naturally occurring molecules, it is expected that such molecules would be present in relatively lesser amounts or have less affinity for the binding domain than the ligand chosen in the purification process (e.g., the chemical moiety capable of binding covalently and non-specifically to the lumen exposed molecules).

Binding domains are useful for detection and/or purification of tissue-specific or organ-specific lumen-exposed molecules. Examples of binding domains include, but are not limited to, biotin, polypeptides, nucleic acids, peptide nucleic acids, organic and inorganic molecules, chelates, a peptide nucleic acid, a naturally occurring or synthetic organic molecule, a chelate (e.g., metal chelating peptides such as polyhistidine tracts and histidine-tryptophan modules that allow purification on immobilized metals), protein A domains that allow purification on immobilized immunoglobulin, and a domain utilized in the FLAGS extension/affinity purification system (Immunex Corp, Seattle, Wash.).

In one embodiment, the binding domain is biotin and its immobilized ligand is avidin or streptavidin. While mammalian cells have significant amounts of naturally biotinylated polypeptides, the use of cleavable membrane impermeant reagents in the methods of the invention allow for the generation of a substantially pure preparation of lumen-exposed molecules and avoid contamination by naturally biotinylated polypeptides.

Cleavable Chemical Moieties

The third domain of the cell membrane impermeable reagent comprises a cleavable chemical moiety that will not cleave under in vivo conditions. It is a “linking domain” situated between the first and second domains. The membrane impermeant reagents of the invention can comprise any cleavable chemical moiety that will not cleave under in vivo conditions and, if a binding domain is present, that can be cleaved without disrupting the binding of the binding domain to a binding domain ligand; such cleavable chemical moieties are well known in the art. For example, disulfide groups can be used; with exemplary mild conditions for cleavage including, e.g., at 37° C. with about 10 to 50 mg/ml dithiothreitol (DTT) at pH 8.5 within 30 minutes disulfides are quantitatively cleaved (the disulfides reduced, in this example); or, disulfides also cleaved with, e.g., about 1% to about 5% β-mercaptoethanol (2-ME), or equivalents.

Alternatively, peptide or oligonucleotide domains can be cleaved by addition of enzymes that recognize specific sequences (e.g., restriction enzymes for specific nucleic acid sequences). For example, the cleavable domain can include a cleavable linker sequences cleavable by endopeptidases, such as, e.g, Factor Xa, enterokinasef (Invitrogen, San Diego, Calif.) plasmin, enterokinase, kallikrem, urokmase, tissue plasminogen activator, clostripain, chymosin, collagenase, Russell's Viper Venom Protease, post-proline cleavage enzyme, V8 protease, thrombin.

The cleavable chemical moiety can also be a disulfide group, a periodate-cleavable glycol, a dithionite-cleavable diazobond, a hydroxylamine-cleavable ester or a base-labile sulfone. The cleavable chemical moiety also can be any chemical entity cleavable by an enzymatic reaction, e.g., a nucleic acid (e.g., an oligonucleotide) that is cleavable by a restriction enzyme, or a peptide domain cleavable by an enzyme, e.g., an endopeptidase. Preferably any such chemical entity cleavable by an enzymatic reaction can be cleaved under mild, non-denaturing conditions. Example of mild conditions includes non-denaturing conditions comprising approximately physiologic pH, about 22° C. to 37° C., physiologic salt conditions, or equivalent conditions. When the cleavable domain is a disulfide group, an exemplary set of mild conditions comprises about 10-mg/ml dithiothreitol (DTT), at pH 9, for about 1 to 2 minutes at about room temperature in a solution equivalent to physiologic salt conditions (to be used if the cleavable moiety is a disulfide, or equivalent, group).

Labeling or Detectable Domains

The invention provides methods of labeling a molecule exposed on a luminal surface of a perfusible space for its detection (and, if desired) utilizing a cell membrane impermeable reagent comprising three domains: a first chemical moiety domain capable of covalently and non-specifically binding to tissue-specific or organ-specific lumen-exposed molecules, a second labeling or detectable domain moiety and a third cleavable chemical domain between said first and second domains.

The labeling domain can be any composition that is detectable (directly or indirectly) or that is capable of specifically binding to a second composition (which can be immobilized, e.g., on a bead). Such compositions include, but are not limited to, various enzymes, prosthetic groups, colorimetric compositions, fluorescent materials, luminescent materials, bioluminescent materials, and radioactive material. Examples of suitable enzymes include horseradish peroxidase, alkaline phosphatase, β-galactosidase, or acetylcholinesterase. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminal. Examples of bioluminescent materials include luciferase, luciferin, and aequorin. See, e.g., U.S. Pat. Nos. 6,022,748; 6,007,994. Radioisotopes or radionucleotides can be used as labeling or detectable moieties, e.g., Sc, Fe, Pb, Ga, Y, Bi, Mn, Cu, Cr, Zn, Ge, Mo, Tc, Ru, In, Sn, Re, Sr, Sm, Lu, Eu, Dy, Sb, W, Po, Ta or TI ions. Exemplary radionucleotides include H-3, S-35,1-125,1-131, P-32, Y-90, Re-188, At-211, Bi-212 and the like. Fluorescent metal ion can be used, e.g., metals of atomic number 57 to 71; e.g., ions of the metals La, Ce, Pr, Nd, Pin, Sin, Eu, Gd, Tb, Dy, Ho, Er, Tin, Yb, and Lu. In another embodiment, the label can comprise a paramagnetic elements suitable for the use in magnetic resonance imaging (MRI) applications, e.g., elements of atomic number 21 to 29, 43, 44 and 57 to 71, e.g., Cr, V, Mn, Fe, Co, Ni, Cu, La, Ce, Pr, Nd, Pin, Sin, Eu, Gd, Tb, Dy, Ho, Er, Tin, Yb and Lu.

In alternative embodiments, the labeling domain of the impermeable reagent can be a polypeptide (e.g., a ligand or epitope), a nucleic acid or a peptide nucleic acid (PNA) (e.g., capable of specifically hybridizing to its complementary sequence), a fluorescent molecule, a colorimetric agent, a radionuclide, a naturally occurring or a synthetic organic molecule or a chelate. In a preferred embodiment, the polypeptide can be a polyhistidine.

In some embodiments, the labeling domain is a fourth domain in a cell membrane impermeable reagent wherein the second domain is a binding domain.

Labeling and Isolating

The compositions here can be used to label and/or isolate molecules exposed on the lumen of a perfusible space, especially tissue-specific or organ-specific molecules. Such molecules (“tagged” molecules) can be attached directly or indirectly to the membrane or cells lining of the perfusible space (e.g., extracellular matrix molecules, deposits or buildups present only in certain pathologic, inflammatory, infectious conditions or at particular stages of development). Examples of lumen-exposed “tagged” molecules include, but are not limited to, polypeptides, lipids, carbohydrates (e.g., polysaccharides), nucleic acids, peptide nucleic acids, etc.

The perfusible space of the present invention can be any vascular vessel (e.g., ventricles, atrium, arteries, arterioles, capillaries, veins, renal artery, lobar artery, interlobar artery, arcuate artery, small interlobular artery, afferent arterioles, arcuate vein, interlobar vein, renal vein, ducts of exocrine and endocrine glands). The perfusible space can also be a lumen of a cerebral spinal fluid (CSF) space. The perfusible space can also be a lumen of a lymphatic vessel, an endocrine or exocrine duct, a pore, or equivalent thereto. The perfusible space can be an ejaculatory duct or prostatic urethra. Furthermore, the cell lining of the perfusible space can be lined with endothelial cells, epithelial cells, or both. In preferred embodiments, the perfusible space is one that belongs to any of the following organs and/or tissues: heart, lung, brain, liver, kidney, colon, pancreas, prostate, central nervous system, skin, digestive tract, and the eye.

Perfusion of a perfusible space can be accomplished by any means known in the art. Such methodologies include, for example, aortic arch flush, as in, e.g., Woods (1999) J. Trauma 47:1028-1036; arterial cannula in the supraceliac aorta, as in e.g., Mishima (1999) Ann. Thorac. Surg. 67:874-875; coaxial catheter systems permitting movement in three dimensions, as in, e.g., Lauer (1999) J. Am. Coll. Cardiol. 34:1663-1670; cardiac catheterization by a transhepatic approach as in, e.g., McLeod (1999) Heart 82:694-696; central venous catheterization as in, e.g., Albuquerque (1998) Curr. Opin. Clin. Nutr. Metab. Care 1:297-304; placement, of central venous catheters as in, e.g., Cavatorta (1999) Clin. Nephrol. 52:191-193, or Ball (1999) Anaesthesia 54:819, and the like. In various embodiment, the methods of the invention comprise perfusion, or infusion, cell membrane impermeable reagents into lymphatic ducts. Such methodologies are well known in the art, e.g., cannulation as in Chuang (1986) J. Surg. Res. 41:563-568; direct cannulation mediastinal lymphatics as in Leeds (1981) Invest. Radiol. 16:193-200; see also, e.g., Tran (1993) Perit. Dial. Int. 13:270-279. Preferably, the cell membrane impermeable reagent is administered (perfused or infused) intrathecally into epithelial lined perfusible spaces, such as, e.g., exocrine and endocrine ducts and pores, respiratory epithelium (e.g., nasal epithelium, bronchi, lungs, sinuses), cerebral spinal fluid space (CSF), digestive tract and colon epithelium (mouth, pharynx, esophagus, stomach, intestines, colon), kidney epithelium (e.g., capsular epithelium and glomerular epithelium), prostate epithelium, bladder, etc. In other embodiments, perfusion or infusion of the cell membrane impermeable reagent is administered (perfused or infused) into endothelial lined perfusible spaces, such as, for example, endothelium of the prostate gland, endothelial cells of the pulmonary artery, glomerular endothelial, and endothelial cells.

The compositions for administration will commonly comprise a buffered solution comprising a cell membrane impermeable reagent in a pharmaceutically acceptable carrier, e.g., an aqueous carrier. A variety of carriers can be used, e.g., buffered saline and the like. These solutions can be sterile, e.g., generally free of undesirable matter. These compositions may be sterilized by conventional, well-known sterilization techniques. The compositions may contain pharmaceutically acceptable auxiliary substances as required to approximate physiological conditions such as pH-adjusting and buffering agents, toxicity adjusting agents and the like, for example, sodium acetate, sodium chloride, potassium chloride, calcium chloride, sodium chloride, sodium lactate and the like. The exact concentration of cell membrane impermeable reagents, and the frequency of administration can also be adjusted by routine determinations.

The cell membrane impermeable reagents of the invention will commonly be administered into a perfusible space of an intact organ or tissue, or into an intact animal in a buffered aqueous solution comprising the cell membrane impermeable reagent. Concentrations of reagent can vary under the circumstances, e.g., from about 0.5 to about 10 mg/ml; optimal buffers and dosages can be determined by routine methods. In one embodiment, two separate cell membrane impermeable reagents are co-administered.

The cell membrane impermeable reagents can be delivered directly or indirectly into a perfusible space by any means known in the art. Examples of methods of administration or delivery of the cell membrane impermeable reagent include, but are not limited to, systemically (e.g., intravenously), regionally, or locally (e.g., intra- or peri-tumoral or intracystic injection) by, e.g., intraarterial, intratumoral, intravenous (IV), parenteral, intra-pleural cavity, topical, oral, or local administration, as subcutaneous, intratracheal (e.g., by aerosol) or transmucosal (e.g., buccal, bladder, vaginal, uterine, rectal, nasal mucosa), intra-tumoral (e.g., transdermal application or local injection). For example, intra-arterial injections can be used to have a “regional effect,” e.g., to focus on a specific organ (e.g., brain, liver, spleen, lungs). For example, intra-hepaatic artery injection can be used to localize delivery of cell membrane impermeable reagents to the liver; or, intra-carotid artery injection to localize delivery of cell membrane impermeable reagents to the brain (e.g., occipital artery, auricular artery, temporal artery, cerebral artery, maxillary artery, etc.). Administration can be made into intact organs or tissues in vivo or in situ into an intact animal.

The perfusible spaces are perused in situ, in vivo, or in vitro with a cell membrane impermeable reagent herein to react the reagent with the lumen exposed molecules. The cell membrane impermeable reagent used for isolation preferably comrpses of three domains wherein (i) a first domain comprising a chemical moiety capable of covalently and non-specifically binding to a molecule exposed on the luminal surface of a cell or tissue lining a perfusible space (including extracellular matrix, connective tissue, and the like) in situ or in vivo, (ii) a second domain comprising a binding domain, and (iii) a third domain situated between the first and second domains linking the first domain to the second domain by a cleavable chemical moiety, wherein the cleavable chemical moiety will not cleave under in vivo conditions.

After perfusion, the tagged molecule is isolated by making an isolate, homogenate, or extract preparation from the cell, tissue, or organ being analyzed. Preparations can be made by any means known in the art. The preparations are then reacted with a ligand that has an affinity for the cell membrane impermeable reagent or more preferably to the binding domain of the cell membrane impermeable reagent. After contacting the reagent-reacted molecules in the isolate, homogenate or extract with the ligand, one or more non-bound molecule or substantially all of the non-bound molecules from the ligand-bound fraction are removed (e.g., by washing or by electrophoresis).

Subsequently, the reagent-reacted molecule is isolated by cleaving the cleavable chemical moiety of the cell membrane impermeable reagent. The condition used for cleaving the cleavable chemical moiety does not dissociate the binding domain from the ligand. Preferably, the condition used for cleaving the cleavable chemical moiety does not denature the reacted and isolated molecule. Therefore, the condition for cleaving the chemical moiety preferably comprises a mild condition, which is reducing and non-denaturing. After cleaving the cleavable chemical moiety, the reagent-reacted molecule can be further isolated by elution from the binding domain and the ligand.

In one example, a cell membrane impermeable reagent comprises three domains (i) a first domain comprising a chemical moiety capable of covalently and non-specifically binding to molecules exposed on the luminal surface of said perfusible space in situ or in vivo, (ii) a second domain comprising a biotin binding domain, and (iii) a third domain comprising a disulfide cleavable chemical moiety situated between the first and second domains linking the first domain to the second domain. The cell membrane impermeable reagent is administered to a perfusible space e.g., in an intact organ or an intact animal to react the cell membrane impermeable reagent with lumen-exposed molecules. The reagent-reacted lumen-exposed molecules are subsequently isolated by contacting the isolate or homogenate with an immobilized avidin or streptavidin molecules and removing substantially all of the non-immobilized molecules. In various embodiments, the ligand can be immobilized, e.g., on a bead, membrane, a gel, a fiber, or the like.

The method of isolating can further comprise the step of comparing the reagent-reacted molecules from different organs or tissues to identify a tissue-specific or organ-specific molecule, wherein the tissue-specific or organ-specific molecule is exposed on the luminal surface of the perfusible space of only one of the compared organs or tissues. A molecule is tissue-specific or organ-specific wherein it appears in some but not all, or one but not all of the tissues.

Alternatively, reagent-reacted molecules from the different tissue states (e.g., at different stages of development, aging, apoptosis, before and/or after exposure to particular hormones, cytokines (e.g., lymphokines), neurotransmitters, insults, drugs, injury, infection, disease, or treatment, etc.) may be compared. The comparison may identify a “state” or “reaction”-specific molecule, wherein such molecules are exposed only on the luminal surface of tissues or organs from one state or reaction. The identification and isolation of state and reaction-specific molecules may be utilized to diagnose and/or treat state-specific targets.

Analysis of Isolated Molecules

This invention provides methods to isolate molecules (e.g., organ- or tissue-specific polypeptides) exposed on a luminal surface of a perfusible space. These molecules, e.g., carbohydrates, lipids, polypeptides, and the like can be analyzed and quantified by any of a number of general means well known to those of skill in the art. These include, e.g., analytical biochemical methods such as NMR, spectrophotometry, electrospray ionization (e.g., Fourier transform ion cyclotron resonance mass spectrometry; see, e.g., U.S. Pat. No. 6,011,260), radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immuno-fluorescent assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting and affinity chromatography.

Polypeptides identified as tissue-specific or organ-specific can be separated or purified from other lumen-exposed molecules in the preparation by methods well known in the art. Such methods include, but are not limited to, ammonium sulfate precipitation, PEG precipitation, immunoprecipitation, standard chromatography, immunochromatography, size exclusion chromatography, ion exchange chromatography, hydrophobic interaction chromatography, affinity chromatography, BPLC two-dimensional electrophoresis, 1D electrophoresis and preparative electrophoresis. These and other well-known methods of protein purification may be found in Guide to Protein Purification (M. V. Deutcher, ed.), Methods Enzymol. vol. 182, Academic Press, San Diego, Calif. (1990). The purity of the protein product obtained can be assessed using techniques such as SDS PAGE.

Purified and partially purified polypeptides that have been identified as tissue-specific or organ-specific can be sequenced using methods well known in the art. If the final step of the purification protocol is electrophoresis, the purified or partially purified band (or spot for a two dimensional electrophoresis) corresponding to the polypeptide of interest can be excised from the gel. The polypeptide is then recovered from the polyacrylamide gel using known techniques such as, electroelution into membrane traps, diffusion out of homogenized gel slices, or homogenization then processing using a Microcon® filter (Millipore). N-terminal amino acid sequence can be obtained by subjecting the purified polypeptide to Edman degradation. In addition, the internal amino acid sequence can be obtained by digesting the polypeptide of interest with proteases or cyanogen bromide. For example, the polypeptide of interest can be trypsinized or subject to digestion with V8 protease. The peptide fragments are then separated by HPLC. The sequence of purified peptide fragments are determined by standard amino acid sequencing methods, such as Edman degradation, digestion with carboxypepsidase Y followed by Matrix Assisted Laser Desorbtion Ionization-Time Of Flight (MALDI-TOF) mass spectrometry or Quadrupole-Time Of Flight (Q-TOF) tandem mass spectrometry.

The amino acid sequences or partial amino acid sequences obtained for the tissue-specific or organ-specific lumen-exposed polypeptides can be used as a query sequence for database searching methods using software such as Basic Local Alignment Search Tool (BLAST). BLAST is a family of programs for database similarity searching. The BLAST family of programs includes: BLASTN, a nucleotide sequence database searching program, BLASTX, a protein database searching program where the input is a nucleic acid sequence; and BLASTP, a protein database searching program. BLAST programs embody a fast algorithm for sequence matching, rigorous statistical methods for judging the significance of matches, and various options for tailoring the program for special situations.

In one example, the N-terminal or internal polypeptide sequences obtained kidney-specific lumen-exposed polypeptides includes SEQ ID NOs.: 17-26, 37, 38, 41, 64, and 66; the N-terminal or internal polypeptide sequences obtained lung-specific lumen-exposed polypeptides includes SEQ ID NO.: 27, 38, 41, 43, and 45; the N-terminal or internal polypeptide sequences obtained colon-specific lumen-exposed polypeptides includes SEQ ID NOs.: 28-29, 48 and 50; the N-terminal or internal polypeptide sequences obtained prostate-specific lumen-exposed polypeptides includes SEQ ID NOs.: 30, and 56-59; the N-terminal or internal polypeptide sequences obtained heart-specific lumen-exposed polypeptides includes SEQ ID NOs.: 43, 45, 74-76, 78, 80, 85, 90-93, 95, and 102; the N-terminal or internal polypeptide sequences obtained brain-specific lumen-exposed polypeptides includes SEQ ID NOs.: 60, 62, 70-71, and 89; and the N-terminal or internal polypeptide sequences obtained pancreas-specific lumen-exposed polypeptides includes SEQ ID NOs.: 48, 50, 52, 54, 103 and 104.

Any and all of the above polypeptides can be used to query a nonredundant protein database (National Center for Biotechnology Information). The identity or similarity of the polypeptide sequence to database sequences can be identified using BLASTP with the default parameters. (Altschul, S. F. et al. Gapped BLAST and PSI-BLAST: A New Generation of Protein Database Search Programs, Nucleic Acid Res. 25: 3389-3402 (1997)).

Alternatively, the peptide sequences that are identified as described herein can be analyzed against protein database sequences using the MS PATTERN ver. 4.0.0 software available from the University of California San Francisco, Protein Prospector internet site (prospector.ucsf.edu). For example, each sequenced fragment can be used as a query sequence against various publicly available protein sequence databases, such as the NCBI non redundant (nr) database, SwissProt and Owl. For each fragment, the database set is restricted to proteins having a molecular mass within about +/−25 kDa of the molecular mass of the protein from which the query fragment is obtained. Further specificity can be obtained by requiring the N-terminal query sequences align near the N-terminus of a matching database sequence. If the N-terminal query sequence matches within 60 amino acids of the N-terminus of a database sequence, the N-terminal portion of the database sequence is further analyzed by using the program SIGNALP to determine the location of any N-terminal signal sequences and cleavage sites.

For each of the sequenced fragments, the first query of the analysis requires that the amino acid sequence of the fragment exactly match a database sequence. If no match is obtained from the first query, successive iterations are performed until a sequence match is obtained for each of the fragments analyzed. A match is considered significant only if the aligned portions of the polypeptide display at least 60% sequence identity, more preferably at least 70% sequence identity, more preferably at least 80% sequence identity and more preferably at least 90% sequence identity. If tryptic sequence fragments are used as query sequences, both sequence fragments are required to match the same database protein at level of at least 60% identity, more preferably at least 70% sequence identity, more preferably at least 80% sequence identity and more preferably at least 90% sequence identity. Those sequence fragments that have less than 60% sequence identity to a polypeptide in the database are considered to be unmatched.

Database searching also provides a method for identifying the polynucleotide sequences that encode the polypeptides identified using BLAST or other equivalent search algorithm. These polynucleotide sequences as well as polynucleotide sequences encoding homologous polypeptides from other species can then be used to design oligonucleotide primers which can be used to obtain a full-length cDNA or a cDNA fragment which encodes the polypeptide of interest or a portion thereof. For peptide sequences which have no database match, a degenerate primer can be designed using the sequenced peptide fragment. Using RACE PCR, the entire full-length cDNA or a portion thereof can be obtained. (See Bertling, W. M., et al. (1993) PCR Methods Appl. 3: 95-99; Frohman, M. A. (1991) Methods Enzymol. 218: 340-362; PCR Protocols: A Guide to Methods and Applications, (M. A. Innis, ed.), Academic Press, San Diego, Calif. (1990)). These polynucleotides can then be sequenced using methods well known in the art.

The polynucleotide sequences obtained using the above methods can be used in further database searching to identify homologous polynucleotide sequences and corresponding homologous polypeptide sequences from other organisms. Homologous polypeptides can also be used for tissue-specific or organ-specific targeting using therapeutic complexes. The homologous polypeptides described herein are those that have both a similar or identical amino acid sequence or a similar or substantially similar biological activity as a tissue-specific or organ-specific lumen-exposed polypeptide identified as described herein. Homologous polypeptides can be from the same of different species. Homologous polypeptides can contain amino acid substitutions, additions or deletions provided that the molecules remain biologically equivalent to the polypeptides that are obtained by the methods described herein.

Homologous polypeptides are proteins that are encoded by polynucleotides that are capable of hybridizing with an oligonucleotide probe that hybridizes with a cDNA sequence that encodes a tissue-specific or organ-specific lumen-exposed polypeptide. Examples of cDNA sequences encoding kidney-specific lumen-exposed polypeptides include SEQ ID NOs.: 2, 4, 6, 8, 10, 12, 39-40, 65, 67, and homologs thereof; cDNA sequences encoding lung-specific lumen-exposed polypeptides include SEQ ID NO.: 14, 114, and homologs thereof; cDNA sequences encoding colon-specific lumen-exposed polypeptides include SEQ ID NO.: 16, 49, 51, and homologs thereof; cDNA sequences encoding heart-specific lumen-exposed polypeptides include SEQ ID NO.: 40-46, 52, and homologs thereof; cDNA sequences encoding brain-specific lumen-exposed polypeptides include SEQ ID NO.: 61, 63, 38, 39, and homologs thereof; cDNA sequences encoding pancreas-specific lumen-exposed polypeptides include SEQ ID NO.: 49, 51, 53, 55, 120-121 and homologs thereof; and cDNA sequences encoding prostate-specific lumen-exposed polypeptides include SEQ ID NOs.: 32, 34, and homologs thereof.

The oligonucleotide probes that bind the above-described polynucleotides can be considerably shorter than the entire sequence, but should be at least 25, preferably at least 40, more preferably at least 100, even more preferably at least 200, and still more preferably at least 400 nucleotides in length. Longer probes can also be used. Both DNA and RNA probes can be used. The probes are labeled for detecting the corresponding gene (for example, with ³²P, ³³p, biotin, or avidin).

The full-length cDNAs encoding the homologous tissue-specific or organ-specific lumen-exposed polypeptides identified as described herein can be obtained by nucleic acid hybridizations methods under moderate stringency conditions. Such methods are well known in the art. (J. Sambrook, E. F. Fritsch, and T. Maniatus, Molecular Cloning, A Laboratory Manual, 2d edition, Cold Spring Harbor, N.Y., (1989)). An example of a hybridization performed at moderate stringency conditions is the hybridization of an oligonucleotide probe to carrier-bound polynucleotides in 6× sodium chloride/sodium citrate (SSC) at about 45° C. followed by one or more washes in 0.2×SSC containing 0.1% SDS at about 42-65° C.

The amino acid sequences of the homologous polypeptides can differ from the amino acid sequence of tissue-specific or organ-specific lumen-exposed polypeptides by an insertion or deletion of one or more amino acid residues and/or the substitution of one or more amino acid residues by different amino acid residues. Preferably, amino acid changes are of a minor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small connector peptide of up to about 20-25 residues; or a small extension that facilitates purification by changing net charge or another function, such as a poly-histidine tract, an antigenic epitope or a binding domain.

Nucleic acid expression vectors, containing a polynucleotide that encodes a tissue-specific or organ-specific lumen-exposed polypeptide or a portion thereof can be constructed. Such expression vectors can include, for example, a polynucleotide encoding a kidney-specific lumen-exposed polypeptide having a nucleic acid sequence selected from the group consisting of SEQ ID NOs.: 2, 4, 6, 8, 10, 12, 39-40, 65, 67, or homologs thereof; a polynucleotide encoding a lung-specific lumen-exposed polypeptide having a nucleic acid sequence of SEQ ID NO.: 14, 114, or homologs thereof; a polynucleotide encoding a colon-specific lumen-exposed polypeptide having a nucleic acid sequence consisting of SEQ ID NO.: 16, 49, 51, or homologs thereof, a polynucleotide encoding a prostate-specific lumen-exposed polypeptide having a nucleic acid sequence selected from the group consisting of SEQ ID NOs.: 32, 34 or homologs thereof; a polynucleotide encoding a pancreas-specific lumen-exposed polypeptide having a nucleic acid sequence selected from the group consisting of SEQ ID NO.: 49, 51, 53, 55, 120-121, or homologs thereof; a polynucleotide encoding a heart-specific lumen-exposed polypeptide having a nucleic acid sequence selected from the group consisting of SEQ ID NOs.: 40-46, 52, or homologs thereof; a polynucleotide encoding a brain-specific lumen-exposed polypeptide having a nucleic acid sequence selected from the group consisting of SEQ ID NOs.: 61, 63, 38, 39, or homologs thereof.

Expression vectors containing a polynucleotide that encodes a polypeptide homologous to a tissue-specific or organ-specific lumen-exposed polypeptide, or portion thereof are also contemplated.

A variety of nucleic acid expression vectors suitable for the expression of tissue-specific or organ-specific lumen-exposed polypeptides are well known in the art. Many of these expression vectors include one or more regulatory sequences that are selected on the basis of the host cells to be used for expression. These regulatory sequences are operably linked to the polynucleotide of interest that is to be expressed. Several of these regulatory sequences, which include promoters, enhancers and other expression control elements, are described in Gene Expression Technology (Goeddel, D. V., ed.), Methods Enzymol. vol. 185, Academic Press, San Diego, Calif. (1990).

It will be appreciated by those of ordinary skill in the art that the design of an expression vector depends on a variety of factors. Some of these factors include, but are not limited to, the choice of the host cell to be transformed, the level of expression of protein desired, the ability to regulate protein expression, localization of the expressed protein, and ease of purification of the expressed protein. Recombinant expression vectors that are useful in the expression of the polypeptides described herein can be introduced into a host cell then induced to produce proteins or peptides, including fusion proteins or peptides, that are encoded by the polynucleotides obtained by the methods described herein.

Recombinant expression vectors can be designed for expression of tissue-specific or organ-specific lumen-exposed polypeptides in prokaryotic or eukaryotic cells. For example, a polypeptide of interest can be expressed in bacterial cells such as E. coli, insect cells (using baculovirus expression vectors), yeast cells or mammalian cells. Suitable host cells are discussed further in Gene Expression Technology (Goeddel, D. V., ed.), Methods Enzymol. vol. 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification (affinity handle). Fusion expression vectors often contain a proteolytic cleavage site that is introduced at the junction of the fusion moiety and the recombinant protein. This cleavage site enables separation of the recombinant protein from the fusion moiety during or subsequent to the purification of the fusion protein. Enzymes useful in facilitating the cleavage of fusion proteins at their cognate recognition sequences include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET lld (Studier et al., Gene Expression Technology (Goeddel, D. V., ed.), Methods Enzymol. vol. 185, Academic Press, San Diego, Calif., pp. 60-89, (1990)). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21(DE3) or HMS174(DE3) from a resident prophage harboring a T7 gnI gene under the transcriptional control of the lacUV 5 promoter.

One strategy that can be used to maximize recombinant protein expression in E. coli is to express the protein in a host bacteria with an impaired capacity to proteolytically cleave the recombinant protein (Gottesman, S., Gene Expression Technology (Goeddel, D. V., ed.), Methods Enzymol. vol. 185, Academic Press, San Diego, Calif., pp. 119-128, (1990)). Another strategy is to alter the nucleic acid sequence of the polynucleotide to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard techniques known in the art.

Vectors that are used for the expression of recombinant proteins in yeast are also useful in the expression of a tissue-specific or organ-specific lumen-exposed polypeptide. Examples of vectors useful for expression in the yeast Saccharomyces cerevisae include pYepSec1 (Baldari, et al., (1987) Embo J. 6:229-234), pMFa (Kujan and Herskowitz, (1982) Cell 30:933-943), pJRY88 (Schultz et al., (1987) Gene 54:113-123), pYES2 (Invitrogen Corporation, San Diego, Calif.), and picZ (Invitrogen Corp, San Diego, Calif.).

Alternatively, the tissue-specific or organ-specific lumen-exposed polypeptide can be expressed in insect cells using baculovirus expression vectors. Examples of baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In cases where expression in mammalian cells is desired, the tissue-specific or organ-specific lumen-exposed polypeptide can be expressed using a mammalian expression vector. Examples of mammalian expression vectors include pCDM8 (Seed, B. (1987) Nature 329:840) and pMT2PC (Kaufmnan et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The host cell into which the expression vector is introduced can be any prokaryotic or eukaryotic cell. The expression vector can be introduced into these cells via conventional transformation or transfection techniques, including but not limited to calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual 2nd, ed, Cold Spring Harbor Laboratory, Cold Spring Harbor Laboratory Press, Cold Spring Harbor, N.Y., 1989), as well as other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding the tissue-specific or organ-specific lumen-exposed polypeptide of interest or can be introduced on a separate vector. Cells stably transfected with the introduced nucleic acid can be identified by drug selection (e.g., cells that have incorporated the selectable marker gene will survive, while the other cells die).

Kits

The invention provides kits that contain the cell membrane impermeable reagents of the invention in suitable buffers for administration (perfusion) to intact organs, tissues or animals. In one embodiment, the kits also contain printed matter setting forth instructions for practicing the methods of the invention, as set forth herein.

The invention provides a kit comprising a cell membrane impermeable reagent comprising three domains: (i) a first domain comprising an active moiety capable of covalently and non-specifically binding to a molecule expressed on the luminal surface of a cell lining a perfusible space in situ or in vivo, (ii) a second domain comprising a binding domain, and, (iii) a third domain comprising a disulfide moiety situated between the first and second domains linking the first domain to the second domain; and printed matter instructing use of the cell membrane impermeable, reagent for administration into a lumen of an intact organ or an intact animal to react the cell membrane impermeable reagent with a molecule expressed on the luminal surface to isolate the reagent-reacted molecule. In one embodiment of the kit, the binding domain of the cell membrane impermeable reagent is biotin and the printed matter instructs isolation of the reagent-reacted molecules by contact with an immobilized avidin or streptavidin molecule and removing substantially all of the non-immobilized molecules. In another embodiment, the printed matter instructs administration into a lumen of an artery, a arteriole, a capillary or a vein.

III. Tissue-Specific or Organ-Specific Lumen-Exposed Molecules

Tissue-specific or organ-specific lumen exposed molecules isolated using the methods disclosed herein can be used to deliver therapeutic agents to specific tissue or tissues of choice. In one aspect, a therapeutic complex comprises a ligand that binds specifically to a target protein or a tissue-specific or organ-specific lumen exposed molecule. Examples of therapeutic complexes include, but are not limited to, antibodies (e.g., polyclonal, monoclonal, humanized) antibody fragments (e.g., Fab, Fab′ and Fab′₂) or single chain Fv. In another aspect, a therapeutic complex comprises a ligand or binding-agent, which binds specifically to a tissue-specific or organ-specific lumen-exposed molecule and furthermore is linked to one or more therapeutic agents to be delivered to the tissue expressing the lumen-exposed molecule. Such therapeutics complex typically comprises of the ligand, a therapeutic moiety (e.g., agents) and a linker linking the therapeutic moiety to the ligand. In a preferred embodiment, tissues targeted or tissues expressing a lumen-exposed molecule include kidney, lung, prostate, colon, brain, heart, pancreas, kidney, gut or any combination thereof. Examples of tissue-specific or organ-specific lumen-exposed molecules, ligands, therapeutic moieties, and linkers are disclosed in U.S. application Ser. No. 10/165,603, which claims priority to U.S. Provisional Application Serial Nos. 60/297,021 and 60/305,117; and in US/PCT 03/10195, which claims priority to U.S. Provisional Application Serial No. 60/369,452, incorporated herein by reference in their entirety for all purposes.

Therapeutic Complex

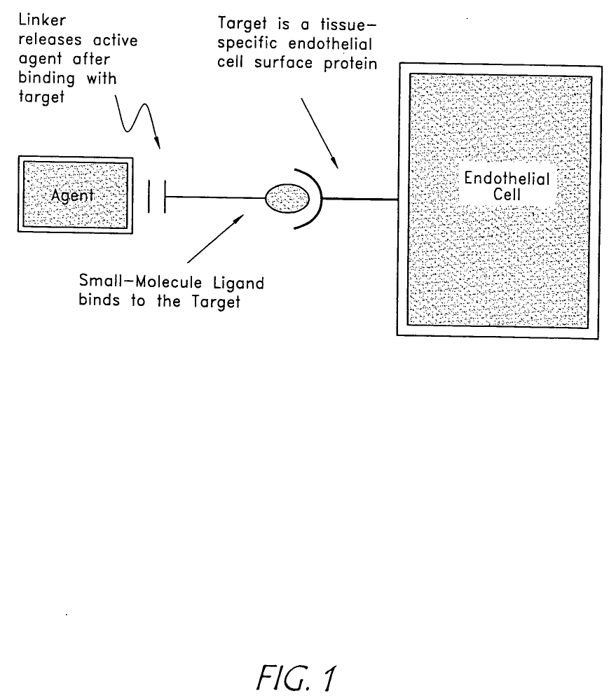

The therapeutic complexes of the invention bind to the target proteins, for example from the pancreas, lung, muscle, intestine, prostate, kidney, and brain to specifically deliver a therapeutic moiety to the tissue or organ of choice. The therapeutic complexes are composed of at least one ligand, a linker, and at least one therapeutic moiety. See FIG. 1. However, the attachment of the three types of components of the therapeutic complex can be envisioned to have a large number of different embodiments, e.g., polyvalent system can be used in which multiple therapeutic agents are linked to a single ligand by any method known in the art. The therapeutic moiety can be one or more of any type of molecule which is used in a therapeutic or diagnostic way. For example, the therapeutic moiety can be an antibiotic which needs to be taken up by a specific tissue. The therapeutic complex can be envisioned to concentrate and target the antibiotic to the tissue where it is needed, thus increasing the therapeutic index of that antibiotic. Alternatively, the therapeutic moiety can be for in vivo or in vitro diagnostic purposes.

In one aspect, the present invention provides for a method of delivering a therapeutic agent to a specific tissue or organ comprising administering a therapeutically effective amount of a therapeutic complex, wherein said therapeutic complex comprises: (i) a ligand which binds to a tissue-specific or organ-specific lumen-exposed molecule, (ii) a therapeutic moiety, and (iii) a linker which links said therapeutic moiety to said ligand. The therapeutic complexes of the present invention bind to the target proteins, for example from the kidney, colon, prostate, heart, pancreas, lung, heart, and brain, to specifically deliver a therapeutic moiety to the tissue or organ of choice. The therapeutic complexes are composed of at least one ligand, a linker, and at least one therapeutic moiety as illustrated in FIG. 1. However, the attachment of the three types of components of the therapeutic complex can be envisioned to have a large number of different embodiments. The therapeutic moiety can be one or more of any type of molecule which is used in a therapeutic or diagnostic way. For example, the therapeutic moiety can be an antibiotic which needs to be taken up by a specific tissue. The therapeutic complex can be envisioned to concentrate and target the antibiotic to the tissue where it is needed, thus increasing the therapeutic index of that antibiotic. Alternatively, the therapeutic moiety can be for diagnostic purposes. Further examples of the use of therapeutic complexes in the specific embodiments of the present invention will be outlined in more detail in the section entitled “Type of Therapeutic Complex Interactions”.

Ligands

A ligand when referring to a therapeutic complex, may be a protein, RNA, DNA, small molecule, peptide nucleic acid, antibody, or any other type of molecule that can bind to target proteins, or more preferably, a lumen-expressed tissue-specific or organ-specific protein. In one embodiment, the ligand is an antibody, or part thereof, which specifically binds to a luminally expressed, tissue-specific or organ-specific molecule. Usually, the ligand recognizes an epitope which does not participate in the binding of a natural ligand. The ligand of the lumen-expressed tissue-specific or organ-specific endothelial protein can be identified by any technique known to one of skill in the art, for example, using a two-hybrid technique, a combinatorial library, or producing an antibody molecule.

In one example, purified tissue-specific or organ-specific lumen-exposed molecules identified by the methods disclosed herein can be utilized to generate antibodies directed to a tissue-specific or organ-specific lumen-exposed molecule of interest. Such antibodies may be utilized as ligands or binding agents. For example, an antibody or binding agent that can bind to a kidney-specific lumen exposed molecule such as CD98, CD108, CD10, CD13, or a combination thereof, may be utilized as a ligand for targeting therapeutic complexes to kidney tissue. Similarly, binding agents and antibodies that bind Ectonucleotide Pyrophosphatase/Phosphodiesterase 5 may be utilized as a ligand for targeting therapeutic complexes to lung tissue. The present invention also contemplates the use of CD73-binding agents or antibodies as colon-specific ligands. And furthermore, it is contemplated by the present invention that Na/K ATPase beta-1 subunit-binding agents and antibodies may provide a prostate-specific ligand. Methods for producing antibodies to tissue-specific or organ-specific molecules are disclosed herein.

The target tissue-specific or organ-specific molecule may be an integral membrane protein (such as a receptor) or may be a ligand itself. Should the tissue-specific or organ-specific molecule be a ligand which binds to a luminally expressed protein, the ligand, or a fragment thereof which exhibits the lumen and tissue-specificity or organ-specificity, is used in the construction of the therapeutic complex of the invention. Alternatively, antibodies, antibody fragments, or antibody complexes specific to, or with similar binding characteristics to, the luminally exposed ligand molecule may be used in the construction of the therapeutic complex of the invention.

Should the tissue-specific or organ-specific lumen-exposed protein (target protein) is a receptor, natural ligands can be identified by one of skill in the art in a number of different ways. For example, a two-hybrid technique can be used. Alternatively, high-throughput screening can be used to identify peptides which can act as ligands. Other methods of identifying ligand are known to one of skill in the art.

In one embodiment, the ligand of the therapeutic complex uses a different epitope than the natural ligand of the receptor target protein, so that there is no competition for binding sites.

In another embodiment, the ligand is an antibody molecule and preferably the antibody molecule has a higher specificity or binds to the tissue-specific or organ-specific lumen-exposed receptor target protein in such a way that it will not be necessary to compete with the natural ligand.

Antibodies and fragments can be made by standard methods (See, for example, E. Harlow et al., Antibodies, A Laboratory Manual, Cold Spring Harbor Laboratory, Cold Spring Harbor, N.Y., 1988). However, the isolation, identification, and molecular construction of antibodies have been developed to such an extent that the choices are almost inexhaustible. Therefore, examples of antibody parts, and complexes will be provided with the understanding that this can only represent a sampling of what is available.

The smallest fragment to bear the antigen-binding site is the Fv portion of an antibody, a 26 kDa heterodimer consisting of the amino-terminal variable domains of the heavy and light chains. (Bird et al. (1988) Science 242:423-426). The antigen-binding moiety can be located in a whole antibody, antibody fragment, or subfragment. Antibodies can be whole immunoglobulin (IgG) of any class, e.g., IgG, IgM, IgA, IgD, IgE, chimeric antibodies or hybrid antibodies with dual or multiple antigen or epitope specificities, or fragments, such as F(ab′).sub.2, Fab′, Fab and the like, including hybrid fragments. Any immunoglobulin or any natural, synthetic, or genetically engineered protein that acts like an antibody by binding to luminally-exposed molecules can be used to target the therapeutic complex.

Preparations of polyclonal antibodies can be made using standard methods which are well known in the art. Antibodies can include antiserum preparations from a variety of commonly used animals, e.g., goats, primates, donkeys, swine, rabbits, horses, hens, guinea pigs, rats, or mice, and even human antisera after appropriate selection and purification. Animal antisera are raised by inoculating the animals with immunogenic epitopes of the tissue-specific or organ-specific lumen-exposed molecules isolated by the methods disclosed herein. The animals are then bled and the serum or an immunoglobulin-containing serum fraction is recovered.

Hybridoma-derived monoclonal antibodies (human, monkey, rat, mouse, or the like) are also suitable for use in the present invention and have the advantage of high specificity. They are readily prepared by what are now generally considered conventional procedures for the immunization of mammals with preparations such as, the immunogenic epitopes of the tissue-specific or organ-specific lumen-exposed molecules isolated by the methods disclosed herein, fusion of immune lymph or spleen cells with an immortal myeloma cell line, and isolation of specific hybridoma clones. More unconventional methods of preparing monoclonal antibodies are not excluded, such as interspecies fusions and genetic engineering manipulations of hypervariable regions, as it is primarily the specificity of the antibodies for the tissue-specific or organ-specific lumen-exposed molecules that affects their utility in the present invention.

In one embodiment, the antibody is a single chain Fv region. Antibody molecules have two generally recognized regions, in each of the heavy and light chains. These regions are the so-called “variable” region, which is responsible for binding to the specific antigen in question, and the so-called “constant” region, which is responsible for biological effector responses such as complement binding, binding to neutrophils and macrophages, etc. The constant regions are not necessary for antigen binding. The constant regions have been separated from the antibody molecule, and variable binding regions have been obtained. Therefore, the constant regions are clearly not necessary for the binding action of the antibody molecule when it is acting as the ligand portion of the therapeutic complex.

The variable regions of an antibody are composed of a light chain and a heavy chain. Light and heavy chain variable regions have been cloned and expressed in foreign hosts, while maintaining their binding ability. Therefore, it is possible to generate a single chain structure from the multiple chain aggregate (the antibody), such that the single chain structure will retain the three-dimensional architecture of the multiple chain aggregate.

Fv fragments which are single polypeptide chain binding proteins having the characteristic binding ability of multi-chain variable regions of antibody molecules, can be used for the ligand of the present invention. These ligands are produced, for example, following the methods of Ladner et al., U.S. Pat. No. 5,260,203, issued Nov. 9, 1993, using a computer based system and method to determine chemical structures. These chemical structures are used for converting two naturally aggregated but chemically separated light and heavy polypeptide chains from an antibody variable region into a single polypeptide chain which will fold into a three dimensional structure very similar to the original structure of the two polypeptide chains. The two regions may be linked using an amino acid sequence as a bridge.

The single polypeptide chain obtained from this method can then be used to prepare a genetic sequence coding therefor. The genetic sequence can then be replicated in appropriate hosts, further linked to control regions, and transformed into expression hosts, wherein it can be expressed. The resulting single polypeptide chain binding protein, upon refolding, has the binding characteristics of the aggregate of the original two (heavy and light) polypeptide chains of the variable region of the antibody.

In a further embodiment, the antibodies are multivalent forms of single-chain antigen-binding proteins. Multivalent forms of single-chain antigen-binding proteins have significant utility beyond that of the monovalent single-chain antigen-binding proteins. A multivalent antigen-binding protein has more than one antigen-binding site, which results in an enhanced binding affinity. The multivalent antibodies can be produced using the method disclosed in Whitlow et al., U.S. Pat. No. 5,869,620 issued Feb. 9, 1999. The method involves producing a multivalent antigen-binding protein by linking at least two single-chain molecules, each single chain molecule having two binding portions of the variable region of an antibody heavy or light chain linked into a single chain protein. In this way the antibodies can have binding sites for different parts of an antigen or have binding sites for multiple antigens.

In one embodiment, the antibody is an oligomer. The oligomer is produced as in PCT/EP97/05897, filed Oct. 24, 1997, by first isolating a specific ligand from a phage-displayed library. Oligomers overcome the problem of the isolation of mostly low affinity ligands from these libraries, by oligomerizing the low-affinity ligands to produce high affinity oligomers. The oligomers are constructed by producing a fusion protein with the ligand fused to a semi-rigid hinge and a coiled coil domain from Cartilage Oligomeric Matrix Protein (COMP). When the fusion protein is expressed in a host cell, it self assembles into oligomers.

Preferably, the oligomers are peptabodies (Terskikh et al., Biochemistry 94:1663-1668 (1997)). Peptabodies can be exemplified as IgM antibodies which are pentameric with each binding site having low-affinity binding, but able to bind in a high affinity manner as a complex. Peptabodies are made using phage-displayed random peptide libraries. A short peptide ligand from the library is fused via a semi-rigid hinge at the N-terminus of the COMP (cartilage oligomeric matrix protein) pentamerization domain. The fusion protein is expressed in bacteria where it assembles into a pentameric antibody which shows high affinity for its target. Depending on the affinity of the ligand, an antibody with very high affinity can be produced.

Preferably the antibody, antibody part or antibody complex of the present invention is produced in humans or is “humanized” (i.e. non-immunogenic in a human) by recombinant or other technology. Such antibodies are the equivalents of the monoclonal and polyclonal antibodies disclosed herein, but are less immunogenic, and are better tolerated by the patient.

Humanized antibodies may be produced, for example, by replacing an immunogenic portion of an antibody with a corresponding, but non-immunogenic portion (i.e. chimeric antibodies) (See, for example, Robinson, et al., International Patent Publication No. PCT/US86/02269; Akira, et al., European Patent Application No. 184,187; Taniguchi, European Patent Application No. 171,496; Morrison, et al., European Patent Application No. 173,494; Neuberger, et al., PCT Application No. WO86/01533; Cabilly, et al., European Patent Application No. 125,023; Better, et al., Science 240:1041-1043 (1988); Liu, et al., Proc. Natl. Acad. Sci. USA 84:3439-3433 (1987); Liu, et al., J. Immunol. 139:3521-3526 (1987); Sun, et al., Proc. Natl. Acad. Sci. USA 84:214-218 (1987); Nishimura, et al., Canc. Res. 47:999-1005 (1987); Wood, et al., Nature 314:446-449 (1985)); Shaw et al., J. Natl. Cancer Inst. 80:1553-1559 (1988)). General reviews of “humanized” chimeric antibodies are provided by Morrison, (Science, 229:1202-1207 (1985)) and by Oi, et al., BioTechniques 4:214 (1986)).

Suitable “humanized” antibodies can be alternatively produced by CDR or CEA substitution (Jones, et al., Nature 321:552-525 (1986); Verhoeyan et al., Science 239:1534 (1988); Bsidler, et al., J. Immunol. 141:4053-4060 (1988).

Other types of antibodies can be generated and used to construct the therapeutic complexes of the invention. For example, chimeric antibodies which comprise portions derived from two different species, such as a human constant region and a murine variable or binding region, can be constructed. The portions derived from two different species can be joined together chemically by conventional techniques or can be prepared as single contiguous proteins using genetic engineering techniques. DNA encoding the proteins of both the light chain and heavy chain portions of the chimeric antibody can be expressed as contiguous proteins. Chimeric antibodies can be constructed as disclosed in International Publication Number WO 93/03151. Binding proteins can also be prepared which are derived from immunoglobulins and which are multivalent and multispecific, such as the “diabodies” described in International Publication No. WO 94/13804.

Antibodies can be purified by methods well known in the art. For example, antibodies can be affinity purified by passing the antibodies over a column to which a tissue-specific or organ-specific lumen-exposed molecule is bound. The bound antibodies can then be eluted from the column, using a buffer with a high salt concentration.

Small molecules are any non-biopolymeric DNA, RNA, organic, or inorganic molecules such as macrocycles, alkene isomers, and many of what is typically thought of as drugs in the pharmaceutical industry. These molecules are often identified through combinatorial processes. In particular, a ligand can be identified using a process called “docking”, an approach to rational drug design which seeks to predict the structure and binding free energy of a ligand-receptor complex given only the structures of the free ligand and receptor. Typically, these small molecules are used to bind to a specific protein and elicit an effect. However, it is envisioned in this context that they would simply be used to bind a specific protein and thus localize the attached drug to the required organs.

Ligands can also be produced, for example, using a library of expression vectors which contain stochastically generated polynucleotide sequences. Host cells containing the expression vectors are cultured so as to produce polypeptides encoded by the polynucleotide sequences. The polypeptides can then be screened for the ability to bind to a tissue-specific or organ-specific lumen-exposed molecule of interest by using protein binding assays known in the art, such as electrophoresis through a non-denaturing gel, column chromatography, the yeast two-hybrid assay, and the like. This method of generating binding molecules is taught in U.S. Pat. No. 5,763,192. Computer-aided molecular design can also be used to generate ligands. (See, Caflisch, A. (1996) J. Comput. Aided Mol. Des. 10:372-96).

Linkers

The “linker” as used herein is any bond, small molecule, or other vehicle which allows the ligand and the therapeutic moiety to be targeted to the same area, tissue, or cell. Preferably, the linker is cleavable.

In one embodiment the linker is a chemical bond between one or more ligands and one or more therapeutic moieties. Thus, the bond may be covalent or ionic. An example of a therapeutic complex where the linker is a chemical bond would be a fusion protein. In one embodiment, the chemical bond is acid sensitive and the pH sensitive bond is cleaved upon going from the blood stream (pH 7.5) to the transcytotic vesicle or the interior of the cell (pH about 6.0). Alternatively, the bond may not be acid sensitive, but may be cleavable by a specific enzyme or chemical which is subsequently added or naturally found in the microenvironment of the targeted site. Alternatively, the bond may be a bond that is cleaved under reducing conditions, for example a disulfide bond. Alternatively, the bond may not be cleavable.

Any kind of acid cleavable or acid sensitive linker may be used. Examples of acid cleavable bonds include, but are not limited to: a class of organic acids known as cis-polycarboxylic alkenes. This class of molecule contains at least three carboxylic acid groups (COOH) attached to a carbon chain that contains at least one double bond. These molecules as well as how they are made and used is disclosed in Shen, et al. U.S. Pat. No. 4,631,190. Alternatively, molecules such as amino-sulfhydryl cross-linking reagents which are cleavable under mildly acidic conditions may be used. These molecules are disclosed in Blattler et al., U.S. Pat. No. 4,569,789.

Alternatively, the acid cleavable linker may be a time-release bond, such as a biodegradable, hydrolyzable bond. Typical biodegradable carrier bonds include esters, amides or urethane bonds, so that typical carriers are polyesters, polyamides, polyurethanes and other condensation polymers having a molecular weight between about 5,000 and 1,000,000. Examples of these carriers/bonds are shown in Peterson, et al., U.S. Pat. No. 4,356,166. Other acid cleavable linkers may be found in U.S. Pat. Nos. 4,569,789 and 4,631,190 or Blattner et al. in Biochemistry 24: 1517-1524 (1984). The linkers are cleaved by natural acidic conditions, or alternatively, acid conditions can be induced at a target site as explained in Abrams et al., U.S. Pat. No. 4,171,563.

Examples of linking reagents which contain cleavable disulfide bonds (reducable bonds) include, but are not limited to “DPDPB”, 1,4-di-[3′-(2′pyridyldithio)propionamido]butane; “SADP”, (N-succinimidyl(4-azidophenyl)1,3′dithiopropionate); “Sulfo-SADP” (Sulfosuccinimidyl (4-azidophenyldithio)propionate; “DSP”—Dithio bis (succinimidylproprionate); “DTSSP”—3,3′-Dithio bis (sulfosuccinimidylpropionate); “DTBP”—dimethyl 3,3′-dithiobispropionimidate-2 HCl, all available from Pierce Chemicals (Rockford, Ill.).

Examples of linking reagents cleavable by oxidation are “DST”—disuccinimidyl tartarate; and “Sulfo-DST”—disuccinimidyl tartarate. Again, these linkers are available from Pierce Chemicals.

Examples of non-cleavable linkers are “Sulfo-LC-SMPT”—(sulfosuccinimidyl 6-[alpha-methyl-alpha-(2-pyridylthio)toluamido}hexanoate; “SMPT”; “ABH”—Azidobenzoyl hydrazide; “NHS-ASA”—N-Hydroxysuccinimidyl-4-azidosalicyclic acid; “SASD”—Sulfosuccinimidyl 2-(p-azidosalicylamido)ethyl-1,3-dithiopropionate; “APDP”—N-(4-[p-azidosalicylamido]butyl)-3′(2′-pyidyldithio) propionamide; “BASED”—Bis-[beta-(4-azidosalicylamido)ethyl] disulfide; “HSAB”—N-hydroxysuccinimidyl-4 azidobenzoate; “APG”—p-Azidophenyl glyoxal monohydrate; “SANPAH”—N-Succimiidyl-6(4′-azido-2′-mitrophenyl-amimo)hexanoate; “Sulfo-SANPAH”—Sulfosuccinimidyl 6-(4′-azido-2′nitrophenylamino)hexanoate; “ANB-NOS”—N-5-Azido-2-nitrobenzyoyloxysuccinimide; “SAND”—Sulfosuccinimidyl-2-(m-azido-o-mitrobenzamido)-ethyl-1,3′-dithiopropionate; “PNP-DTP”—p-nitrophenyl-2-diazo-3,3,3-trifluoropropionate; “SMCC”—Succinimidyl 4-(N-maleimidomethyl)cyclohexane-1-carboxylate; “Sulfo-SMCC”—Sulfosuccinimidyl 4-(N-maleimidomethyl)cyclohexane-1-carboxylate; “MBS”—m-Maleimidobenzoyl-N-hydroxysuccinimide ester; “sulfo-MBS”-m-Maleimidobenzoyl-N-hydroxysulfosuccinimide ester; “SIAB”—N-Succinimidyl(4-iodoacetyl)aminobenzoate; “Sulfo-SIAB”—N-Sulfosuccinimidyl(4-iodoacetyl)aminobenzoate; “SMPB”—Succinimidyl 4-(p-malenimidophenyl)butyrate; “Sulfo-SMPB”—Sulfosuccinimidyl 4-(p-malenimidophenyl)butyrate; “DSS”—Disuccinimidyl suberate; “BSSS”—bis(sulfosuccinimidyl) suberate; “BMH”—Bis maleimidohexane; “DFDNB”—1,5-difluoro-2,4-dinitrobenzene; “DMA”—dimethyl adipimidate 2 HCl; “DMP”—Dimethyl pimelimidate-2HCl; “DMS”—dimethyl suberimidate-2-HCl; “SPDP”—N-succinimidyl-3-(2-pyridylthio)propionate; “Sulfo-HSAB”—Sulfosuccinimidyl 4-(p-azidophenyl)butyrate; “Sulfo-SAPB”—Sulfosuccinimidyl 4-(p-azidophenylbutyrate); “ASIB”-1-p-azidosalicylamido)-4-(iodoacetamido)butane; “ASBA”—4-(p-Azidosalicylamido)butylamine. All of these linkers are available from Pierce Chemicals.

In another embodiment the linker is a small molecule such as a peptide linker. In one embodiment the peptide linker is not cleavable. In a further embodiment the peptide linker is cleavable by base, under reducing conditions, or by a specific enzyme. In one embodiment, the enzyme is indigenous. Alternatively, the small peptide may be cleavable by a non-indigenous enzyme which is administered after or in addition to the therapeutic complex. Alternatively, the small peptide may be cleaved under reducing conditions, for example, when the peptide contains a disulfide bond. Alternatively, the small peptide may be pH sensitive. Examples of peptide linkers include: poly(L-Gly), (Poly L-Glycine linkers); poly(L-Glu), (Poly L-Glutamine linkers); poly(L-Lys), (Poly L-Lysine linkers). In one embodiment, the peptide linker has the formula (amino acid)ₙ, where n is an integer between 2 and 100, preferably wherein the peptide comprises a polymer of one or more amino acids.

In a further embodiment, the peptide linker is cleavable by proteinase such as one having the sequence Gly-(D)Phe-Pro-Arg-Gly-Phe-Pro-Ala-Gly-Gly (SEQ ID. NO: 35) (Suzuki, et al. 1998, J. Biomed. Mater. Res. Oct. 42(1):112-6). This embodiment has been shown to be advantageous for the treatment of bacterial infections, particularly Pseudomonas aeruginosa. Gentamicin or an alternate antibiotic is cleaved only when the wounds are infected by Pseudomonas aeruginosa because there is significantly higher activity of thrombin-like proteinase enzymes then in non-infected tissue.

In a further embodiment the linker is a cleavable linker comprising, poly(ethylene glycol) (PEG) and a dipeptide, L-alanyl-L-valine (Ala-Val), cleavable by the enzyme thermolysin. This linker is advantageous because thermolysin-like enzyme has been reported to be expressed at the site of many tumors. Alternatively, a 12 residue spacer Thr-Arg-His-Arg-Gln-Pro-Arg-Gly-Trp-Glu-Gln-Leu (SEQ ID NO: 36) may be used which contains the recognition site for the protease furin (Goyal, et al. Biochem. J. 2000 Jan. 15; 345 Pt 2:247-254).

The chemical and peptide linkers can be bonded between the ligand and the therapeutic moiety by techniques known in the art for conjugate synthesis, i.e. using genetic engineering, or chemically. The conjugate synthesis can be accomplished chemically via the appropriate antibody by classical coupling reactions of proteins to other moieties at appropriate functional groups. Examples of the functional groups present in proteins and utilized normally for chemical coupling reactions are outlined as follows. The carbohydrate structures may be oxidized to aldehyde groups that in turn are reacted with a compound containing the group H₂NNH—R (wherein R is the compound) to the formation of a C═NH—NH—R group. The thiol group (cysteines in proteins) may be reacted with a compound containing a thiol-reactive group to the formation of a thioether group or disulfide group. The free amino group (at the amino terminus of a protein or on a lysine) in amino acid residues may be reacted with a compound containing an electrophilic group, such as an activated carboxy group, to the formation of an amide group. Free carboxy groups in amino acid residues may be transformed to a reactive carboxy group and then reacted with a compound containing an amino group to the formation of an amide group.

The linker may alternatively be a liposome. Many methods for the preparation of liposomes are well known in the art. For example, the reverse phase evaporation method, freeze-thaw methods, extrusion methods, and dehydration-rehydration methods. See Storm, et al. PSTT 1:19-31 (1998).

The liposomes may be produced in a solution containing the therapeutic moiety so that the substance is encapsulated during polymerization. Alternatively, the liposomes can be polymerized first, and the biologically active substance can be added later by resuspending the polymerized liposomes in a solution of a biologically active substance and treating with sonication to affect encapsulation of the therapeutic moiety. The liposomes can be polymerized in the presence of the ligand such that the ligand becomes a part of the phospholipid bilayer. In one embodiment, the liposome contains the therapeutic moiety on the inside and the ligand on the outside.

The liposomes contemplated in the present invention can comprise a variety of structures. For example, the liposomes can be multilamellar large vesicles (MLV), oligolamellar vesicles (OLV), unilamellar vesicles (UV), small unilamellar vesicles (SUV), medium sized unilamellar vesicles (MUV), large unilamellar vesicles (LUV), giant unilamellar vesicles (GUV), or multivesicular vesicles (MVV). Each of these liposome structures is well known in the art. See Storm, et al. PSTT 1:19-31 (1998).

In one embodiment, the liposome is a “micromachine” that avulses pharmaceuticals for example by the application of specific frequency radio waves. In another embodiment, the liposomes can be degraded such that they will release the therapeutic moiety in the targeted cell, for example, the liposomes may be acid or alkaline sensitive, or degraded in the presence of a low or high pH, such that the therapeutic moiety is released within the cell. Alternatively, the liposomes may be uncharged so that they may be taken up by the targeted cell. The liposomes may also be pH sensitive or sensitive to reducing conditions.

One type of liposome which may be advantageously used in the present invention is that identified in Langer et al., U.S. Pat. No. 6,004,534, issued Dec. 21, 1999. In this application a method of producing modified liposomes which are prepared by polymerization of double and triple bond-containing monomeric phospholipids is disclosed. These liposomes have surprisingly enhanced stability against the harsh environment of the gastrointestinal tract. Thus, they have utility for oral and/or mucosal delivery of the therapeutic moiety. It has also been shown that the liposomes may be absorbed into the systemic circulation and lymphatic circulation. The liposomes are generally prepared by polymerization (i.e., radical initiation or radiation) of double and triple bond-containing monomeric phospholipids.

In other embodiments of the present invention, the linker can also be a liposome having a long blood circulation time. Such liposomes are well known in the art. See U.S. Pat. Nos. 5,013,556; 5,225,212; 5,213,804; 5,356,633; and 5,843,473. Liposomes having long blood circulation time are characterized by having a portion of their phospholipids derivatized with polyethylene glycol (PEG) or other similar polymer. In some embodiments, the end of the PEG molecule distal to the phospholipid may be activated so a to be chemically reactive. Such a reactive PEG molecule can be used to link a ligand to the liposome. One example of a reactive PEG molecule is the maleimide derivative of PEG described in U.S. Pat. No. 5,527,528.

In yet other embodiments, the linker may be a microcapsule, a nanoparticle, a magnetic particle, and the like (Kumar, J. Pharm. Sci., May-August 3(2) 234-258, 2000; and Gill et al., Trends Biotechnol. Nov. 18(11):469-79, 2000), with the lipophilic therapeutic moiety on or in the container, and the container functioning as the linker in the therapeutic complex.

Alternatively, the linker may be a photocleavable linker. For example, a 1-2-(nitrophenyl)-ethyl moiety can be cleaved using 300 to 360 nm light (see Pierce catalog no. 21332ZZ). It can be envisioned that the photocleavable linker would allow activation and action of the drug in an even more specific area, for example a particular part of the organ. The light could be localized using a catheter into the vessel. Alternatively, light may be used to localize treatment to a specific part of the digenstive tract and the light may be manipulated through a natural orifice to the area. Alternatively, the light can be surgically manipulated to the area.

Alternatively, the linker may not be cleavable, but the therapeutic moiety or ligand is. An example of this is when the therapeutic moiety is a prodrug and the enzyme, which cleaves the prodrug, is administered with the therapeutic complex. Alternatively, the enzyme is part of the therapeutic complex or indigenous and the prodrug is administered separately. Preferably, the enzyme or prodrug, which is administered separately, is administered within about 48 hours of the first administration. Alternatively, the prodrug or enzyme, which is administered separately, is administered between about 1 minute and 24 hours of the first administration, more preferably the prodrug or enzyme, which is administered separately, is administered between about 2 minutes and 8 hours. The prodrug or enzyme, which is administered separately, may be readministered at a later date and may continue to be administered until the effect of the drug is not longer needed or until the enzymatic cleavage of all of the drug is effected.

Therapeutic Moieties

The “therapeutic moiety” could be any chemical, molecule or complex, which effects a desired result. Examples of therapeutic moieties include but are not limited to anti-neoplastic agents, contrast agents, toxins, radionucleotide agents, paramagnetic agents, immunosuppressive agents, antisense oligonucleotides, and protein agents including surfactants and clotting proteins. Preferably, a therapeutic moiety is preferably lipophilic, which will help it enter the targeted cell.

It can be envisioned that the therapeutic moiety can be any chemotherapeutic agent, alkylating agents (nitrogen mustards, ethylenimines, alkyl sulfonates, nitrosoureas, and triazenes), antimetabolites (folic acid analogs such as methotrexate, pyrimidine analogs, and purine analogs), natural products and their derivatives (antibiotics, alkaloids, enzymes), hormones (adrenocorticosteroids, progestins, estrogens), antagonists, and other compositions that can act as an anti-neoplastic agent. Alternatively, the therapeutic moiety can be an antisense oligonucleotide which acts as an anti-neoplastic agent, or a protein which activates apoptosis in a neoplastic cell.

Other examples of therapeutic moieties include neuroeffectors, anesthetics, anti-inflammatories, blood-pressure modulators, anti-protozoan, anti-bacterial, anti-fungal, toxins, anti-coagulants, vitamins, signaling labels, chromogenic labels, non-enzymatic labels, catalytic labels, chemiluminescent labels, and prodrugs.

Antineoplastic agents include, for example, alkylating agents, antibiotic agents, anti-metabolic agents, biologic agents, hormonal agents, and plant-derived agents.

Alkylating agents are polyfunctional compounds that have the ability to substitute alkyl groups for hydrogen ions. These compounds react with phosphate, amino hydroxyl, sulfhydryl, carboxyl, and imidazole groups, and can lead to an abnormal base pairing and interference with DNA replication, transcription of RNA and disruption of DNA function. Thus, alkylating agents are cell cycle phase-nonspecific agents bc they exert their activity independently of the specific phase of the cell cycle. Examples of alkylating agents include but are not limited to chlorambucil, cyclophosphamide, ifosfamide, mechloreethamine, melphalan, uracil mustard, thiotepa, busulfan, carmustine, lomustine, streptozocin, altretamine, dacarbazine, procarbazine, carboplatin, and cisplatin.

Antibiotic agents that are effective against a variety of human tumors and may be used as therapeutic moieties include for example anthracyclines such as, doxorubicin, daunorubicin, epirubicin and idarubicin; mitomycin C, bleomycin, dactinomycin, and plicamycin. Doxorubicin is preferably targeted for lung tissue and/or prostate tissue as it is a therapeutic agent for both lung carinoma and prostatic carcinoma. Doxorubicin may also be used to target other soft-tissue with carcinoma. Epirubicin is preferably used as a therapeutic moiety when targeting the GI tract such as in the treatment of GI carcinoma. Idarubicin can be used as a therapeutic moiety for a variety of solid tumors. Mitomycin C is preferably used as a therapeutic moiety when targeting colon tissue and/or lung tissue as in the treatment of colorectal and lung carcinoma.

Antimetabolic agents are a large group of anticancer drugs that can interfere with the metabolic processes necessary for the physiology and proliferation of cancer cells. The major groups of antimetabolic agents are the antifols, the purine analogues, and the pyrimidine analogues. Antifols include compounds such as methotrexate. Purine analogues include compounds such as hydroxyurea and mercaptopurine. Pyrimidine analogues include compounds such as fluorouracil, floxuridine, cytarabine, pentostatin, fludarabine phosphate, cladribine and gemcitabine. Other antimetabolic agents in clued leucovorin, hydroxyurea and asparaginase. Methotrexate is a preferred antimetabolic agent used in treatment of lung cancer. On the other hand, fluorouracil is preferably used when targeting pancreatic and/or colon tissue for the treatment of colon and pancreatic cancer. Agemcitabine is another antimetabolic agent that is preferably used to treat locally advanced or metastatic pancreatic cancer.

Biological agents are biological reagents that may elicit tumor regression. Examples of biological agents include interleukins (IL) (e.g., IL-1, IL-2, IL-4, IL-6, IL-7, and IL-12); interferons (e.g., IFN α); bacillus Calmette-Guerin, levamisole, colony-stimulating factors (CSFs) (e.g., erthtopoietin, granulocyte-CSF, and macrophage colony stimulatin gfactor), octreotide and its analogues (e.g., somatostatin, prosomatostatin and preprosomatostatin), and retinoids (e.g., retinoic acid and isotretinoin).

Hormonal agents can be used to affect the growth and proliferation of their target organs (e.g., ovaries and prostate). Hormonal agents regulate such hormones as estrogen, progestins, and androgens. Examples of antiestrogens include toremifene and raloxifene; examples of antiandrogens include bicalutamide and nilutamide; and examples of aromatase inhibitors anastrozole and tetrazole. Hormonal agents preferably used as therapeutic moieties in the treatment of prostate cancer include diethylstilbestreol, flutamide, goserelin acetate, ketoconazole, leuprolide, bicalutamide, and nilutamide.

Plant derived agents may also be used to target cancer tissues. Examples of plant-derived chemotherapeutic agents include alkaloids, such as vincristine, vinblastine, vindesine, vinzolidine and vinorelbine; taxenes, such as paclitaxel and docetaxel; epipodophylotoxins, such as etoposide and teniposide; and camptothecin and its derivative, including topotecan and irinotecan. For lung cancer, preferred plant-derived agents include vincristine, vinblastine, topotecan, docetaxe, and vinorelbine. Such agents can be targeted to lung tissue using therapeutic complex that will bind lung tissue.

The contrast agents may be any type of contrast agent known to one of skill in the art. The most common contrast agents basically fall into one of four groups; X-ray reagents, radiography reagents, magnetic resonance imaging agents, and ultrasound agents. The X-ray reagents include ionic, iodine-containing reagents as well as non-ionic agents such as Omnipaque (Nycomed) and Ultravist (Schering). Radiographic agents include radioisotopes as disclosed below. Magnetic Resonance Imaging reagents include magnetic agents such a Gadolinium and iron-oxide chelates. Ultrasound agents include microbubbles of gas and a number of bubble-releasing formulations.

The radionucleotide agents, like all other agents, may be diagnostic or therapeutic. Examples of radionucleotide agents that are generally medically useful include: Y, Ln, Cu, Lu, Tc, Re, Co, Fe and the like such as ⁹⁰Y, ¹¹¹Ln, ⁶⁷Cu, ⁷⁷Lu, ⁹⁹Tc and the like, preferably trivalent cations, such as ⁹⁰Y and ¹¹¹Ln. Radionucleotide agents that are suitable for imaging organs and tissues in vivo via diagnostic gamma scintillation photemetry include the following: γ-emitting radionuclides: ¹¹¹Ln, ^{113m}Ln, ⁶⁷Ga, ⁶⁸Ga, ^{99m}Tc, ⁵¹Cr, ¹⁹⁷Hg, ²⁰³Hg, ¹⁶⁹Yb, ⁸⁵Sr, and ⁸⁷Sr. The preparation of chelated radionucleotide agents that are suitable for binding by Fab′ fragments is taught in U.S. Pat. No. 4,658,839 (Nicoletti et al.). Examples of therapeutic radionucleotide agents that are suitable β-emitters include ⁶⁷Cu, ¹⁸⁶Rh, ¹⁸⁸Rh, ¹⁸⁹Rh, ¹⁵³Sm, ⁹⁰Y, and ¹¹¹Ln.

Paramagnetic agents are paramagenetic metal ions suitable for use as imaging agents in MRI include the lanthanide elements of atomic number 57-70, or the transition metals of atomic numbers 21-29, 42 or 44. U.S. Pat. No. 4,647,447 (Gries et al.) teaches MRI imaging via chelated paramagnetic metal ions.

Antisense oligonucleotides have a potential use in the treatment of any disease caused by over-expression of a normal gene, or expression of an aberrant gene. Antisense oligonucleotides can be used to reduce or stop expression of that gene. Examples of oncogenes which can be treated with antisense technology and references which teach specific antisense molecules which can be used include: c-Jun and cFos (U.S. Pat. No. 5,985,558); HER-2 (U.S. Pat. No. 5,968,748) E2F-1 (Popoff, et al. U.S. Pat. No. 6,187,587), SMAD 1-7 (U.S. Pat. Nos. 6,159,697; 6,013,788; 6,013,787; 6,013,522; and 6,037,142), and Fas (Dean et al. U.S. Pat. No. 6,204,055). Other oligonucleotide agents include interfering RNA, mRNA, cDNA, and genomic DNA for gene therapy.

Proteins which may be used as therapeutic agents include apoptosis inducing agents such as pRB and p53 which induce apoptosis when present in a cell (Xu et al. U.S. Pat. No. 5,912,236), and proteins which are deleted or underexpressed in disease such as erythropoietin (Sytkowski, et al. U.S. Pat. No. 6,048,971).

The therapeutic moiety can be any type of neuroeffector, for example, neurotransmittors or neurotransmitter antagonists may be targeted to an area where they are needed without the wide variety of side effects commonly experienced with their use.

The therapeutic moiety can be an anesthetic such as an opioid, which can be targeted specifically to the area of pain. Side effects, such as nausea, are commonly experienced by patients using opioid pain relievers. The method of the present invention would allow the very specific localization of the drug to the area where it is needed, such as a surgical wound or joints in the case of arthritis, which may reduce the side effects.

The therapeutic moiety can be an anti-inflammatory agent such as histamine, H¹-receptor antagonists, and bradykinin. Alternatively, the anti-inflammatory agent can be a non-steroidal anti-inflammatory such as salicylic acid derivatives, indole and indene acetic acids, and alkanones. Alternatively, the anti-inflammatory agent can be one for the treatment of asthma such as corticosteroids, cromollyn sodium, and nedocromil. The anti-inflammatory agent can be administered with or without the bronchodilators such as B²-selective andrenergic drugs and theophylline.

The therapeutic moiety can be a diuretic, a vasopressin agonist or antagonist, angiotensin, or renin, which specifically affect a patient's blood pressure.

The therapeutic moiety can be any pharmaceutical used for the treatment of protozoan infections such as tetracycline, clindamycin, quinines, chloroquine, mefloquine, trimethoprimsulfamethoxazole, metronidazole, and oramin. The ability to target pharmaceuticals or other therapeutics to the area of the protozoal infection is of particular value due to the very common and severe side effects experienced with these antibiotic pharmaceuticals.

The therapeutic moiety can be any anti-bacterial such as sulfonamides, quinolones, penicillins, cephalosporins, aminoglycosides, tetracyclines, chloramphenicol, erythromycin, isoniazids and rifampin.

The therapeutic moiety can be any pharmaceutical agent used for the treatment of fungal infections such as amphotericins, flucytosine, miconazole, and fluconazole.

The therapeutic moiety can be any pharmaceutical agent used for the treatment of viral infections such as acyclovir, vidarabine, interferons, ribavirin, zidovudine, zalcitabine, reverse transcriptase inhibitors, and protease inhibitors. It can also be envisioned that virally infected cells can be targeted and killed using other therapeutic moieties, such as toxins, radioactive atoms, and apoptosis-inducing agents.

The therapeutic moiety can be chosen from a variety of anticoagulant, anti-thrombolyic, and anti-platelet pharmaceuticals.

It can be envisioned that diseases resulting from an over- or under-production of hormones can be treated using such therapeutic moieties as hormones (growth hormone, androgens, estrogens, gonadotropin-releasing hormone, thyroid hormones, adrenocortical steroids, insulin, and glucagon). Alternatively, if the hormone is over-produced, antagonists or antibodies to the hormones may be used as the therapeutic moiety.

Various other possible therapeutic moieties include vitamins, enzymes, and other under-produced cellular components and toxins such as diptheria toxin or botulism toxin.

Alternatively, the therapeutic moiety may be one that is typically used in in vitro diagnostics. Thus, the ligand and linker are labeled by conventional methods to form all or part of a signal generating system. The ligand and linker can be covalently bound to radioisotopes such as tritium, carbon 14, phosphorous 32, iodine 125 and iodine 131 by methods well known in the art. For example, ¹²⁵I can be introduced by procedures such as the chlorogine-T procedure, enzymatically by the lactoperoxidase procedure or by the prelabeled Bolton-Hunter technique. These techniques plus others are discussed in H. Van Vunakis and J. J. Langone, Editors, Methods in Enzymology, Vol. 70, Part A, 1980. See also U.S. Pat. No. 3,646,346, issued Feb. 29, 1972, and Edwards et al., U.S. Pat. No. 4,062,733, issued Dec. 13, 1977, respectively, for further examples of radioactive labels.

Therapeutic moieties also include chromogenic labels, which are those compounds that absorb light in the visible ultraviolet wavelengths. Such compounds are usually dyestuffs and include quinoline dyes, triarylmethane dyes, phthaleins, insect dyes, azo dyes, anthraquimoid dyes, cyanine dyes, and phenazoxonium dyes.

Fluorogenic compounds can also be therapeutic moieties and include those which emit light in the ultraviolet or visible wavelength subsequent to irradiation by light. The fluorogens can be employed by themselves or with quencher molecules. The primary fluorogens are those of the rhodamine, fluorescein and umbelliferone families. The method of conjugation and use for these and other fluorogens can be found in the art. See, for example, J. J. Langone, H. Van Vunakis et al., Methods in Enzymology, Vol. 74, Part C, 1981, especially at page 3 through 105. For a representative listing of other suitable fluorogens, see Tom et al., U.S. Pat. No. 4,366,241, issued Dec. 28, 1982, especially at column 28 and 29. For further examples, see also U.S. Pat. No. 3,996,345.

These non-enzymatic signal systems are adequate therapeutic moieties for the present invention. However, those skilled in the art will recognize that an enzyme-catalyzed signal system is in general more sensitive than a non-enzymatic system. Thus, for the instant invention, catalytic labels are the more sensitive non-radioactive labels.

Catalytic labels include those known in the art and include single and dual (“channeled”) enzymes such as alkaline phosphatase, horseradish peroxidase, luciferase, β-galactosidase, glucose oxidase (lysozyme, malate dehydrogenase, glucose-6-phosphate dehydrogenase) and the like. Examples of dual (“channeled”) catalytic systems include alkaline phosphatase and glucose oxidase using glucose-6-phosphate as the initial substrate. A second example of such a dual catalytic system is illustrated by the oxidation of glucose to hydrogen peroxide by glucose oxidase, which hydrogen peroxide would react with a leuco dye to produce a signal generator. (A further discussion of catalytic systems can be found in Tom et al., U.S. Pat. No. 4,366,241, issued Dec. 28, 1982 (see especially columns 27 through 40). Also, see Weng et al., U.S. Pat. No. 4,740,468, issued Apr. 26, 1988, especially at columns 2 and columns 6, 7 and 8.

The procedures for incorporating enzymes into the instant therapeutic complexes are well known in the art. Reagents used for this procedure include glutaraldehyde, p-toluene diisocyanate, various carbodiimide reagents, p-benzoquinone m-periodate, N,N¹-ophenylenedimaleimide and the like (see, for example, J. H. Kennedy et al., Clin. Chim Acta 70, 1 (1976)). As another aspect of the invention, any of the above devices and formats may be provided in a kit in packaged combination with predetermined amounts of reagents for use in assaying for a tissue-specific or organ-specific endothelial protein.

Chemiluminescent labels are also applicable as therapeutic moieties. See, for example, the labels listed in C. L. Maier, U.S. Pat. No. 4,104,029, issued Aug. 1, 1978.

The substrates for the catalytic systems discussed above include simple chromogens and fluorogens such as para-nitrophenyl phosphate (PNPP), β-D-glucose (plus possibly a suitable redox dye), homovanillic acid, o-dianisidine, bromocresol purple powder, 4-alkyl-umbelliferone, luminol, para-dimethylaminolophine, paramethoxylophine, AMPPD, and the like.

Depending on the nature of the label and catalytic signal producing system, one would observe the signal by irradiating with light and observing the level of fluorescence; providing for a catalyst system to produce a dye, fluorescence, or chemiluminescence, where the dye could be observed visually or in a spectrophotometer and the fluorescence could be observed visually or in a fluorometer; or in the case of chemiluminescence or a radioactive label, by employing a radiation counter. Where the appropriate equipment is not available, it will normally be desirable to have a chromophore produced which results in a visible color. Where sophisticated equipment is involved, any of the techniques are applicable.

Alternatively, the therapeutic moiety can be a prodrug. A prodrug is converted into a corresponding pharmaceutical agent by a change in the chemical environment or by the action of a discrete molecular agent, such as an enzyme. Preferably, the therapeutic moiety is administered with the specific molecule needed for conversion. Alternatively, the prodrug can be cleaved by a natural molecule found in the microenvironment of the target tissue. Alternatively, the prodrug is pH sensitive and converted upon change in environment from the blood to the cell or vesicle (Greco et al., J. Cell. Physiol. 187:22-36, 2001).

The concept of prodrugs is well known in the art and is used herein in a similar manner. For example, the therapeutic complexes may have a prodrug attached as a therapeutic moiety which can be converted either by the subsequent injection of a non-indigenous enzyme, or by an enzyme found in the tissue of choice. Alternatively, the therapeutic moiety may be the enzyme which is needed to convert the prodrug.

For example, the enzyme β-lactamase may be a part of the therapeutic complex and the prodrug (i.e., doxocillin) is subsequently added and, because the β-lactamase is only found in the targeted tissue, the doxocillin is only unmasked in that area. Unfortunately, neoplastic tissues usually share the enzyme repertoire of normal tissues, making the use of an indigenous enzyme less desirable. However, it can be envisioned that diseased tissues, particularly those diseased by pathogens, may be producing an enzyme specific to the pathogen which is infecting the tissue and this could be used to design an effective prodrug treatment which would be very specific to the infected tissue. For example, a prodrug which is converted by a viral enzyme (i.e., HBV) could be used with a liver-specific antiviral therapeutic complex to get very specific antiviral effect because the prodrug would only be converted in the microenvironment containing the virus.

Therefore, in one embodiment, a “ligand-enzyme” therapeutic complex is used in combination with the unattached prodrug. The prodrug is cleaved by an enzyme and enters the cell. Preferably, the prodrug is hydrophilic, limiting its ability to cross the endothelial barrier, while the (cleaved) drug is lipophilic, enhancing its ability to distribute into the selected tissue. Alternatively, a “ligand-prodrug” is used as the therapeutic complex in combination with the administration of an unattached non-indigenous enzyme or an indigenous enzyme. The prodrug is cleaved by the enzyme, thus, separated from the therapeutic wherein lipophilic qualities allow it to distribute into selected tissue.

Two of the advantages of the prodrug approach include bystander killing and amplification. One problem with the previous use of antibodies or immunoconjugates in the treatment of cancer was that they were inefficiently taken up by the cells and poorly localized. However, when using a prodrug treatment, because a single molecule of enzyme can convert more than one prodrug molecule the chance of uptake is increased or amplified considerably. In addition, as the active drug diffuses throughout the tumor, it provides a bystander effect, killing or otherwise effecting the therapeutic action on antigen-negative, abnormal cells. Although this bystander effect may also effect normal cells, they will only be those in the direct vicinity of the tumor or diseased organ.

A number of prodrugs have been widely used for cancer therapy and are presented below as examples of prodrugs which can be used in the present invention (Greco et al., J. Cell. Phys. 187:22-36, 2001; and Konstantinos et al., Anticancer Research 19:605-614, 1999). However, it is to be understood that these are some of many examples of this embodiment of the invention.

The most well-studied enzyme/prodrug combination is Herpes simplex virus thymidine kinase (HSV TK) with the nucleotide analog GCV. GCV and related agents are poor substrates for the mammalian nucleoside monophosphate kinase, but can be converted (1000 fold more) efficiently to the monophosphate by TK from HSV 1. Subsequent reactions catalyzed by cellular enzymes lead to a number of toxic metabolites, the most active ones being the triphosphates. GCV-triphosphate competes with deoxyguanosine triphosphate for incorporation into elongating DNA during cell division, causing inhibition of the DNA polymerase and single strand breaks.

The system consisting of cytosine deaminase and 5-fluorocytosine (CD and 5-FC respectively) is similarly based on the production of a toxic nucleotide analog. The enzyme CD, found in certain bacteria and fungi but not in mammalian cells, catalyses the hydrolytic deamination of cytosine to uracil. It can therefore convert the non-toxic prodrug 5-FC to 5-fluorouracil (5-FU), which is then transformed by cellular enzymes to potent pyrimidine antimetabolites (5-FdUMP, 5-FdUTP, and 5-FUTP). Three pathways are involved in the induced cell death: thymidylate synthase inhibition, formation of (5-FU) RNA and of (5-FU) DNA complexes.

The mustard prodrug CB1954 [5-(aziridin-1-yl)-2,4-dinitrobenzamide] is a weak monofunctional alkylator, but it can be efficiently activated by the rodent enzyme DT diaphorase into a potent DNA cross-linking agent. However, the human enzyme DT diaphorase shows a low reactivity with the prodrug, causing side effects. This problem was overcome when the E. coli enzyme nitroreductase (NTR) was found to reduce the CB1954 prodrug 90 times faster then the rodent DT diaphorase. The prodrug was converted to an alkylating agent which forms poorly repairable DNA crosslinks.

The oxazaphosphorine prodrug cyclophosphamide (CP) is activated by liver cytochrome P450 metabolism via a 4-hydroxylation reaction. The 4-hydroxy intermediate breaks down to form the bifunctional alkylating toxin phosphoramide mustard, which leads to DNA cross-links, G₂-M arrest and apoptosis in a cycle-independent fashion.

In the enzyme/prodrug systems described so far the prodrug is converted to an intermediate metabolite, which requires further catalysis by cellular enzymes to form the active drug. The decreased expression of or total lack of these enzymes in the target cells would lead to tumor resistance. The bacterial enzyme carboxypeptidase G2 (CPG2), which has no human analog, is able to cleave the glutamic acid moiety from the prodrug 4-[2-chloroethyl)(2-mesyloxyethyl)amino]benzoic acid without further catalytic requirements.

The reaction between the plant enzyme horseradish peroxidase (HRP) and the non-toxic plant hormone indole-3-acetic acid (IAA) has been analyzed in depth, but not yet completely elucidated. At neutral pH, IAA is oxidized by HRP-compound I to a radical cation, which undergoes scission of the exocyclic carbon-carbon bond to yield the carbon-centered skatolyl radical. In the presence of oxygen, the skatolyl radical rapidly forms a peroxyl radical, which then decays to a number of products, the major ones being indole-3-carbinol, oxindole-3-carbinol and 3-methylene-2-oxindole. In anoxic solution, decarboxylation of the radical cation can still take place and the carbon-centered radical preferentially reacts with hydrogen donors.

As can readily be seen, the prodrug/enzyme systems advantageously use an enzyme which is not produced by human cells to provide specificity. However, it can readily be seen by one of skill in the art that a human enzyme which is specifically produced in a particular organ or cell type could also be used to achieve this specificity, with the advantage that it would not be immunogenic.

Finally, heterogeneity could be circumvented by the application of a “cocktail” of conjugates constructed with the same enzyme and a variety of antibodies directed against different organ-associated antigens or different antigenic determinants of the same antigen.

Uses of Therapeutic Complexes

The therapeutic complexes herein can be used for the diagnosis, prognosis and treatment of various diseases and in particular tissue-specific or organ-specific diseases. Examples of such tissues and diseases are as follows.

In one embodiment, the therapeutic complex may be used to treat or prevent conditions, which affect the brain. Examples of such diseases include but are not limited to: anxiety, bacterial infections, viral infections, fungal and parasitic infections, epilepsy, depression, schizophrenia, bipolar disorder, headaches and migranes, neurosis, brain cancer, Parkinson's disease, Alzheimer's disease and other forms of dementia, prion-related diseases, stroke, ataxia, multiple sclerosis, meningitis, brain abscess, and Wernicke's disease or other metabolic disorders.

In a further embodiment, the therapeutic complex may be used to treat or prevent conditions, which affect the lungs. Examples of such diseases include but are not limited to: asthma, acute respiratory disorder, acute bronchitis, atelectasis, bacterial infection (i.e. S. pneumoniae, M. tuberculosis), brinchiectasis, chronic obstructive pulmonary disease, cystic fibrosis, emphysema, fungal and parasitic infection (i.e. Pneumocystis carinii), lung cancer (i.e., adenocarcinoma, bronchioloalveolar carcinoma, large cell carcinoma, and squamous cell carcinoma), lung transplant rejection, pneumonia, pulmonary adenomatosis, pulmonary embolism, pulmonary hypertension, pulmonary thromboembolism, pulmonary edema, severe acute respiratory syndrome, lung abscess, and viral infections (i.e. Hantavirus).

In a further embodiment, the therapeutic complex may be used to treat or prevent conditions, which affect the pancreas. Examples of such diseases include but are not limited to: parasitic infections, pancreatic cancer, chronic pancreatitis, and pancreatic insufficiency, endocrine tumors, and diabetes.

In one embodiment, the therapeutic complex may be used to treat or prevent conditions, which affect the kidney. Examples of such diseases include but are not limited to: acute renal failure, albuminuria, Alport syndrome, amyloidosis, proteinuria, analgesic-associated kidney disease, bacterial infections, Berger's disease, bile nephrosis, bladder and renal cell cancer, chronic renal failure, congenital nephrotic syndrome, cyst, cystine stones, cystitis, edema, enuresis, Ellis type II, focal and segmental hyalinosis, focal glomerulonephritis, Formad's kidney, fungal and parasitic infections, glomerulosclerosis, Goodpasture's syndrome, hypertension, hypervolemia, hypercalciuria, hyperoxaluria, IgA nephropathy, incontinence, interstitial nephritis, kidney transplant rejection, kidney cancer, lupus nephritis, membranoproliferative glomerulonephritis, membranous nephropathy, mesangial proliferative glomerulonephritis, nephrogenic diabetes insipidus, nephropathy, nephrogenic diabetes insipidus, nephrolithiasis, nephrolithiasis, nil disease, polycystic kidney disease, poststreptococcal glomerulonephritis, proteinuria, pyelonephritis, rapidly progressive glomerulonephritis, renal allograft rejection, renal artery stenosis, renal cell carcinoma, reflux nephropathy, renal cell carcinoma, renal cysts, renal osteodystrophy, renal tubular acidosis, renal vein thrombosis, struvite stone, systemic lupus erythematosus, thrombotic thrombocytopenic purpura, transitional cell cancer, uremia, urolithiasis, vasculitis, vesico-ureteric reflux, viral infections, Wegener's granulomatosis, and Wilm's tumor.

In one embodiment, the therapeutic complex may be used to treat or prevent conditions, which affect the muscles. Examples of such diseases include but are not limited to: muscular dystrophy, polymyositis, arthritic diseases, rhabdomyosarcoma, polymyositis, disorders of glycogen storage, and soft tissue sarcomas.

In one embodiment, the therapeutic complex may be used to treat or prevent conditions, which affect the gut or intestine, including the colon. Examples of such diseases include but are not limited to: acute colitis, adenocarcinoma, cancer, carcinoid tumor of colon, collagenous colitis, colorectal cancer, Crohn's disease, cryptosporidiosis, colon cancer, diverticulosis of colon, dysentery, gastroenteritis, giardiasis, inflammatory bowel disease, intestinal parasite ascaris lumbricoides, irritable bowel syndrome, ischemic colitis, leiomyosarcoma of colon, peptic ulcer, pneumatosis intestinalis, polyposis coli, pseudomembranous colitis, squamous cell carcinoma of anus, toxic megacolon, tubulovillous adenoma, ulcerative colitis, tumors of the small intestine and villous adenoma.

In one embodiment, the therapeutic complex may be used to treat or prevent conditions, which affect the prostate. Examples of such diseases include but are not limited to: bening prostate hyperplasia, prostatatis, and prostate cancer.

In one embodiment, the therapeutic complex may be used to treat or prevent conditions, which affect the heart. Examples of such conditions include but are not limited to: stroke, intimal hyperplasia, atherosclerosis, arteriosclerosis, heart murmur, arterial fibrillation, congentical heart disease, coronary heart disease, long QT syndrome, and chronic rejection of heart transplant.

In a further embodiment, the therapeutic complex may be used as a diagnostic of disease or tissue type or to quantify or identify the tissue-specific luminally expressed protein. For example, diagnosis of tissue-specific or organ-specific diseases can be made by detecting the presence and level of tissue-specific or organ-specific lumen-exposed molecules that are present in a disease state. Similarly, treatment and prevention of tissue-specific or organ-specific diseases can be achieved by targeting therapeutics to the diseased tissue or organ.

The cells bearing target proteins interact with the therapeutic complex in two general ways, by transcytosis and passive diffusion. These interactions allow the therapeutic complex to interact directly with the vascular endothelial cell bearing the target protein, become enmeshed in the endothelial matrix containing said endothelial cell, or cross through the endothelial matrix into the encapsulated tissue or organ.

Transcytosis occurs when, after attachment of the complex with the target protein on the endothelial cell, the therapeutic complex is transcytosed across the vasculature into the endothelial matrix tissue or endothelial cell of choice. Preferably, the binding of the ligand to the target protein will stimulate the transport of the therapeutic complex across the endothelium within a transcytotic vesicle. During transcytosis, the conditions within the microenvironment of the vesicle are more highly acidic and can be used to selectively cleave the therapeutic moiety. For this to happen, preferably, the linker should be pH sensitive, so as to be cleaved due to the change in pH upon going from the blood stream (pH 7.5) to transcytotic vesicles or the interior of the cell (pH 6.0) such as the acid sensitive linkers disclosed. Alternatively, a separate linker may not be necessary when the bond between the ligand and the therapeutic moiety is itself acid sensitive.

In passive diffusion, the ligand in the complex may attach to the exterior cell membrane, following which there is release of the therapeutic moiety, which crosses into the endothelial cell or tissue by passive means, but there is no entry of the entire therapeutic complex into the cell. Preferably, the therapeutic agent is released in high concentrations in microproximity to the endothelium within the specific target tissue. These higher concentrations are expected to result in relatively greater concentrations of the drug reaching the target tissue versus systemic tissues.

The therapeutic complexes may be taken up by the cell and stay within the cell or cellular matrix or may cross into the organs and become diffuse within the organ.

The therapeutic complexes of the present invention advantageously bind to a target protein on a specific tissue or organ and can be used for a number of desired outcomes. In one embodiment, the therapeutic complexes are used to keep toxic substances in a specific environment, allowing for a more specific targeting of a therapeutic moiety to that environment and preventing systemic effects of the therapeutic moiety. In addition, a lower concentration of the substance would be needed for the same effect.

In some embodiments, a therapeutic complex is kidney-specific. A kidney-specific therapeutic complex comprises of (i) a ligand that binds to a molecule that is exposed on the luminal surface of kidneys but not other tissue or organs; (ii) a therapeutic moiety; and (iii) a linker that links the ligand to the therapeutic moiety. In preferred embodiments, the ligand of the kidney-specific therapeutic complex can binds to a polypeptide having an amino acid sequence selected from the group consisting of SEQ ID NOs: 1, 3, 5, 7, 9, 11, 17-26, 37, 38, 41, 64, 66, or homolog thereof.

Kidney-specific therapeutic complexes can be used to diagnose, prevent, and/or treat kidney diseases. In particular, kidney-specific therapeutic complexes can be used to diagnose any kidney disease that is associated with the expression of lumen-exposed molecules. In addition, kidney-specific therapeutic complexes can be used to treat and/or diagnose any kidney disease by targeting therapeutics to the kidneys. Kidney diseases that can be treated and/or prevented by the present invention include, but are not limited to, acute renal failure, albuminuria, Alport syndrome, amyloidosis, proteinuria, analgesic-associated kidney disease, congenital nephrotic syndrome, cyst, cystine stones, cystitis, glomerulosclerosis, Goodpasture syndrome, hypercalciuria, hyperoxaluria, IgA nephropathy, interstitial nephritis, kidney cancer, lupus nephritis, membranoproliferative glomerulonephritis, membranous nephropathy, nephrogenic diabetes insipidus, nephrolithiasis, polycystic kidney disease, proteinuria, pyelonephritis, renal cell carcinoma, renal cysts, renal osteodystrophy, renal tubular acidosis, renal vein thrombosis, struvite stone, transitional cell cancer, uremia, urolithiasis, vasculitis, vesicoureteral, Wegener's granulomatosis, and Wilm's tumor.

The therapeutic moiety attached to the ligand will depend upon the disease being treated or diagnosed. For example, to treat kidney cancer such as renal cell carcinoma, a therapeutic moiety such as radionucleotides for targeted radiotherapy and/or immuno-modulators (e.g., interferon and IL-2) may be linked to the same or different ligands. In some embodiments, more than one therapeutic moiety may be linked to the same exact ligand moiety. While it has been shown that most individuals experience sever side effects to immuno-modulators administered systemically, the present invention reduces such side effects by directly targeting the diseased tissue.

In some embodiments, a therapeutic complex is specific to the lungs. Such lung-specific therapeutic complex preferably comprises, (i) a ligand that can bind to a molecule exposed on the luminal surface of lungs but not other tissues or organs; (ii) a therapeutic moiety; and (iii) a linker that links the ligand to the therapeutic moiety. In preferred embodiments, the ligand of the lung-specific therapeutic complex can bind to a polypeptide having an amino acid sequence of SEQ ID NO: 13, 27, 38, 40, 41, 42, 43, 45, or homologs thereof.

Lung-specific therapeutic complexes can be used to diagnose, prevent, and treat various lung-associated diseases. Such diseases include, but are not limited to, bacterial infections (i.e. S. pneumoniae, M. tuberculosis), viral infections (i.e. Hantavirus), fungal and parasitic infections (i.e. Pneumocystis carinii), asthma, lung cancer (i.e., adenocarcinoma, bronchioloalveolar carcinoma, large cell carcinoma, and squamous cell carcinoma), emphysema, lung transplant rejection, cystic fibrosis, pulmonary hypertension, pulmonary thromboembolism, pulmonary edema, and viral infections (i.e. Hantavirus).

As discussed above, the therapeutic moiety attached to the ligand will depend upon the disease being diagnosed or treated. For example, in treating lung cancer, such as squamous cell carcinoma and large cell carcinoma, the therapeutic moiety may be a radionucleotides or an antineoplastic agent. In addition, therapeutic complexes may be administered to a patient diagnosed with lung cancer using markers such as, e.g., CA242, TPA, NSE and CEA. See, e.g., Zhonghua Jie He He Hu Xi Za Zhi. 1999 May; 22(5):271-3.

In some embodiments, colon-specific therapeutic complexes may be used to diagnose, prevent or treat diseases that affect the colon and/or gastrointestinal tract (GI). Such colon-specific therapeutic complexes include (i) a ligand that binds to a colon-specific molecule exposed on the luminal surface of the colon; (ii) a therapeutic moiety; and (iii) a linker that links the ligand to the therapeutic moiety. Preferably, the colon-specific molecule is a polypeptide having an amino acid sequence of SEQ ID NOs: 15, 28-29, 48 or homologs thereof.

Examples of diseases that affect the colon and/or GI and that may be diagnosed, prevented, or treated by the colon-specific therapeutic complexes include, but are not limited to, acute colitis, adenocarcinoma, cancer, carcinoid tumor of colon, collagenous colitis, colorectal cancer, Crohn's disease, cryptosporidiosis, colon cancer, diverticulosis of colon, dysentery, gastroenteritis, giardiasis, inflammatory bowel disease, intestinal parasite ascaris lumbricoides, irritable bowel syndrome, ischemic colitis, leiomyosarcoma of colon, peptic ulcer, pneumatosis intestinalis, polyposis coli, pseudomembranous colitis, squamous cell carcinoma of anus, toxic megacolon, tubulovillous adenoma, ulcerative colitis, tumors of the small intestine and villous adenoma.

Furthermore, the invention herein contemplates the use of prostate-specific therapeutic complexes for the diagnosis, prevention and treatment of diseases associated with the prostate. Examples of such diseases include but are not limited to benign prostatic hyperplasia (BPH), prostatitis and prostate cancer.

Prostate-specific therapeutic complexes have: (i) a ligand that binds to a prostate-specific lumen-exposed molecule; (ii) a therapeutic moiety; and (iii) a linker that links the ligand to the therapeutic moiety. In preferred embodiments, the prostate-specific lumen-exposed molecule is a polypeptide having an amino acid sequence selected from the group consisting of SEQ ID NO: 30, 31, 33, 56-59, or homologs thereof. For example, a therapeutic complex may comprise of a ligand that is an antibody that specifically binds a portion of SEQ ID NO: 30, 31, 33, 56-59, or homologs thereof. The antibody may then be linked to a therapeutic moiety such as a radionucleotide or an antineoplastic agent by a cleavable or a non-cleavable linker. Such therapeutic complex may be utilized in the treatment and/or prevention of BPH, prostatitis and prostate cancer.

In another embodiment, the invention herein can be used to diagnose or treat a condition associated with the pancreas. Examples of such conditions include, but are not limited to: bacterial infections, viral infections, fungal and parasitic infections, epilepsy, schizophrenia, bipolar disorder, headaches and migranes, neurosis, depression, brain cancer, Parkinson's disease, Alzheimer's disease and other forms of dementia, prion-related diseases, stroke, ataxia, multiple sclerosis, meningitis, brain abscess, and Wernicke's disease or other metabolic disorders.

Pancreatic-specific therapeutic complexes have: (i) a ligand that binds to a pancreas-specific lumen-exposed molecule; (ii) a therapeutic moiety; and (iii) a linker that links the ligand to the therapeutic moiety. In preferred embodiments, the pancreatic-specific lumen-exposed molecule is a polypeptide having an amino acid sequence selected from the group consisting of SEQ ID NOs: 48, 50, 52, 54, 103, 104, or homologs thereof.

In another embodiment, the invention herein can be used to diagnose or treat a condition associated with the brain. Examples of such conditions include, but are not limited to: anxiety, bacterial infections, viral infections, fungal and parasitic infections, epilepsy, depression, schizophrenia, bipolar disorder, headaches and migranes, neurosis, brain cancer, Parkinson's disease, Alzheimer's disease and other forms of dementia, prion-related diseases, stroke, ataxia, multiple sclerosis, meningitis, brain abscess, and Wernicke's disease or other metabolic disorders.

Brain-specific therapeutic complexes have: (i) a ligand that binds to a brain-specific lumen-exposed molecule; (ii) a therapeutic moiety; and (iii) a linker that links the ligand to the therapeutic moiety. In preferred embodiments, the brain-specific lumen-exposed molecule is a polypeptide having an amino acid sequence selected from the group consisting of SEQ ID NOs: 60, 62, 70-71, 89, or homologs thereof.

In another embodiment the compositions herein can be used to diagnoses or to treat a condition associated with the heart. A heart associated condition includes, for example: stroke, intimal hyperplasia, atherosclerosis, arteriosclerosis, heart murmur, arterial fibrillation, congentical heart disease, coronary heart disease, long QT syndrome, and chronic rejection of heart transplant.

Heart-specific therapeutic complexes have: (i) a ligand that binds to a heart-specific lumen-exposed molecule; (ii) a therapeutic moiety; and (iii) a linker that links the ligand to the therapeutic moiety. In preferred embodiments, the heart-specific lumen-exposed molecule is a polypeptide having an amino acid sequence selected from the group consisting of SEQ ID NOs: 43, 45, 74-76, 78, 80, 85, 90-93, 95, 102, or homologs thereof.

In a further embodiment, the therapeutic complex is used to keep substances from getting into tissues. The therapeutic moiety might be used to block receptors that if activated would cause further harm to the surrounding tissue.

In a further embodiment the therapeutic complex is used to replace a substance, such as a surfactant protein, or a hormone which is in some way dysfunctional or absent from a specific tissue

In one embodiment, the present invention provides a method of determining the presence and/or concentration of a kidney-specific lumen-exposed molecule by administering in vitro, in vivo or in situ to the kidney or kidney tissue a therapeutic complex with a ligand that binds to a polypeptide having an amino acid sequence selected from the group consisting of SEQ ID NOs: 1, 3, 5, 7, 9, 11, 17-26, 37, 38, 41, 64, 66 and homologs thereof. After administering the therapeutic complex, bound complex is identified and quantified.

In another embodiment, the present invention provides a method of determining the presence and/or concentration of a lung-specific lumen-exposed molecule by administering in vitro, in vivo or in situ to the lung or lung tissue a therapeutic complex with a ligand that binds to a polypeptide having an amino acid sequence selected from the group consisting of SEQ ID NO: 13, 27, 38, 40, 41, 42, 43, 45, or homologs thereof. After administering the therapeutic complex, bound complex is identified and quantified.

In another embodiment, the present invention provides a method of determining the presence and/or concentration of a colon-specific lumen-exposed molecule by administering in vitro, in vivo or in situ to the colon or colon tissue a therapeutic complex with a ligand that binds to a polypeptide having an amino acid sequence of SEQ ID NO: 15, 28-29, 48, or any derivatives, portions, or homologs thereof. After administering the therapeutic complex, bound complex is identified and quantified.

In a further embodiment, the present invention provides a method of determining the presence and/or concentration of prostate-specific lumen-exposed molecule by administering in vitro, in vivo or in situ to the prostate or prostate tissue a therapeutic complex with a ligand that binds to a polypeptide having an amino acid sequence selected from the group consisting of SEQ ID NOs: 30, 31, 33, 56-59, or homologs thereof. After administering the therapeutic complex, bound complex is identified and quantified.

In another embodiment, the present invention provides a method of determining the presence and/or concentration of brain-specific lumen-exposed molecule by administering in vitro, in vivo or in situ to the brain or brain tissue a therapeutic complex with a ligand that binds to a polypeptide having an amino acid sequence selected from the group consisting of SEQ ID NOs: 60, 62, 70-71, 89, or homologs thereof. After administering the therapeutic complex, bound complex is identified and quantified.

In a further embodiment, the present invention provides a method of determining the presence and/or concentration of heart-specific lumen-exposed molecule by administering in vitro, in vivo or in situ to the heart or heart tissue a therapeutic complex with a ligand that binds to a polypeptide having an amino acid sequence selected from the group consisting of SEQ ID NOs: 43, 45, 74-76, 78, 80, 85, 90-93, 95, 102, or homologs thereof. After administering the therapeutic complex, bound complex is identified and quantified.

In a further embodiment, the present invention provides a method of determining the presence and/or concentration of pancreatic-specific lumen-exposed molecule by administering in vitro, in vivo or in situ to the pancreas or pancreatic tissue a therapeutic complex with a ligand that binds to a polypeptide having an amino acid sequence selected from the group consisting of SEQ ID NOs: 48, 50, 52, 54, 103, 104, or homologs thereof. After administering the therapeutic complex, bound complex is identified and quantified.

Administration of the Therapeutic Complexes

The therapeutic complexes of the present invention are said to be “substantially free of natural contaminants” if preparations which contain them are substantially free of materials with which these products are normally and naturally found.

The therapeutic complexes include antibodies, and biologically active fragments thereof, (whether polyclonal or monoclonal) which are capable of binding to tissue-specific luminally-expressed molecules. Antibodies may be produced either by an animal, or by tissue culture, or recombinant DNA means.

In administering to a patient a therapeutic complex, the dosage administered will vary depending upon such factors as the patient's age, weight, height, sex, general medical condition, previous medical history, and the like. In addition, the dosage will vary depending on the therapeutic moiety and the desired effect of the therapeutic complex. As discussed below, the therapeutically effective dose can be lowered if the therapeutic complex is administered in combination with a second therapeutic agent or additional therapeutic complexes. As used herein, one compound is said to be co-administered with a second compound if the administration of the two compounds is in such proximity of time that both compounds can be detected at the same time in the patient's serum.

The therapeutic complexes and/or pharmaceutical compositions herein may be administered by any means including but not limited to, orally, parenterally by inhalation, topically, rectally, ocularly nasally, buccally, vaginally, sublingually, transbuccally, liposomally, via an implanted reservoir (e.g., patch or stent) or via local delivery (e.g., by catheter). The term “parenteral” as used herein includes subcutaneous, intracutaneous, intravenous, intramuscular, intra-articular, intra-adipose, intra-arterial, intrasynovial, intrasternal, intrathecal, intra-vagina, intra-rectal, intralesional, intra-ocular, and intracranial injection or infusion techniques. When the compositions herein are administered via injection, the injection may be by continuous infusion, or by single or multiple boluses.

Preferably, the pharmaceutical compositions are administered locally to effected area or tissue. Localized administration is preferably made by microinjection, topically, or parenterally.

The therapeutic complex may be administered either alone or in combination with one or more additional therapeutic agents. Additional therapeutic agents include, for example, additional therapeutic complexes, alkylating agents, antibiotic agents, antimetabolic agents, biological agents, plant-derived agents, immunosuppressive agents (especially to a recipient of an organ or tissue transplant), chemotherapeutic agents, or other pharmaceutical agents, depending on the therapeutic result which is desired. The administration of such compound(s) may be for either a “prophylactic” or “therapeutic”.

A composition is said to be “pharmacologically acceptable” if its administration can be tolerated by a recipient patient. Such an agent is said to be administered in a “therapeutically effective amount” if the amount administered is physiologically significant. A typical range is 0.1 μg to 500 mg/kg of therapeutic complex per the amount of the patient's weight. One or multiple doses of the therapeutic complex may be given over a period of hours, days, weeks, or months as the conditions suggest. An agent is physiologically significant if its presence results in a detectable change in the physiology of a recipient patient. The term “pharmaceutically effective amount” refers to an amount effective in treating or ameliorating an IL-1 mediated disease in a patient. The term “pharmaceutically acceptable carrier, adjuvant, or excipient” refers to a nontoxic carrier, adjuvant, or excipient that may be administered to a patient, together with a compound of the preferred embodiment, and which does not destroy the pharmacological activity thereof. The term “pharmaceutically acceptable derivative” means any pharmaceutically acceptable salt, ester, or salt of such ester, of a compound of the preferred embodiments or any other compound, which upon administration to a recipient, is capable of providing (directly or indirectly) a compound of the preferred embodiment. Pharmaceutical compositions of this invention comprise any of the compounds of the present invention, and pharmaceutically acceptable salts thereof, with any acceptable carrier, adjuvant, excipient, or vehicle.

The therapeutic complex of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (18^{th} ed., Gennaro, Ed., Mack, Easton Pa. (1990)). In order to form a pharmaceutically acceptable composition suitable for effective administration, such compositions will contain an effective amount of the therapeutic complex, together with a suitable amount of carrier vehicle.

Additional pharmaceutical methods may be employed to control the duration of action. Controlled release preparations may be achieved through the use of polymers to complex or absorb the therapeutic complex. Alternatively, it is possible to entrap the therapeutic complex in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions. Such techniques are disclosed in Remington's Pharmaceutical Sciences (1990).

### EXAMPLES

The following example is offered to illustrate, but not to limit the claimed invention.

### Example 1

### Localization of the Therapeutic Moiety to Tissue Using a Brain-Specific, Luminally Expressed Protein, CD71

CD71, or transferrin receptor, is known to be exposed on the luminal surface of the endothelium in only one tissue: the brain. This molecule was found to exist only in the brain preparation and not in any other tissues using the instant methods, confirming the ability of the method to identify tissue specific endothelial proteins.

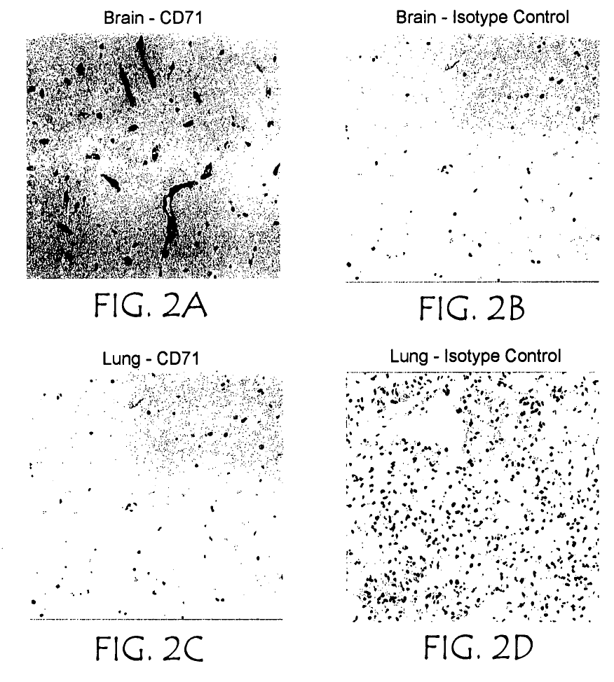

To demonstrate the ability to use the tissue-specific endothelial expression of a protein to selectively deliver an agent to a particular tissue, an antibody to the rat CD71 was used (BD Pharmingen, San Diego, Calif., catalog number 22191). CD71 is a luminally exposed endothelial protein specific to the brain. The rat amino acid and nucleotide sequences are Genbank Accession Nos. AAA42273 and M58040 (SEQ ID NOs:60 and 61), the human amino acid and nucleotide sequences are Genbank Accession Nos. AAH01188 and B0001188 (SEQ ID NOs:62 and 63). The antibody was injected into the tail vein of a rat. Another antibody with a similar isotype but different specificity was injected into another rat as a control. The antibody used as an isotype control was an anti-albumin antibody (IgG2) that was produced by Target Protein Technologies. After 30 minutes, the rats were sacrificed and tissue sections were made from a number of organs from each rat. Each tissue was then analyzed by immunohistochemistry for the presence of the antibodies. FIGS. 2A-D show the immunohistochemistry of tissue sections from a rat which was injected with either CD71 or a control antibody. FIG. 2A is brain from a rat injected with CD71, FIG. 2B is brain from a rat injected with the control antibody, FIG. 2C is lung from a rat injected with CD71, FIG. 2D is lung from a rat injected with the control antibody. These results demonstrate that the anti-CD71 antibody localized to the capillaries of the brain, and to no other tissue. This is particularly advantageous in that it is often difficult to find therapeutics which can cross the blood-brain barrier.

In a follow-up experiment, a toxin was coupled to the anti-CD71 antibody. The toxin used was the Ricin A chain (Sigma, Catalog number L9514). This was coupled to the antibody by adding a biotin with a disulfide-containing linker (Pierce, catalog number 21331) to both the ricin and the antibody. The two were then coupled by the addition of Nuetravidin (Pierce, catalog number 31000), which bound both biotins, thus forming a complex of the ricin and antibody. The in vivo localization experiment was repeated using the toxin-antibody complex. In this case, the antibody not only facilitated the localization of the toxin to the vasculature of the brain, but presumably also its entry into the tissue via transcytosis. Once in the tissue, the toxin elicited an inflammatory response in the brain, a reaction typically seen for any toxin introduced into the brain. No inflammatory response was seen in any other sectioned tissue.

A human CD71-specific antibody is available from BD Pharmingen and usable for the production of a human therapeutic complex.

In Examples 2-6, a number of other tissue-specific luminally expressed proteins were identified and used to produce therapeutic complexes.

### Example 2

### Identification and Sequencing of Rat Dipgptidyl Peptidase IV

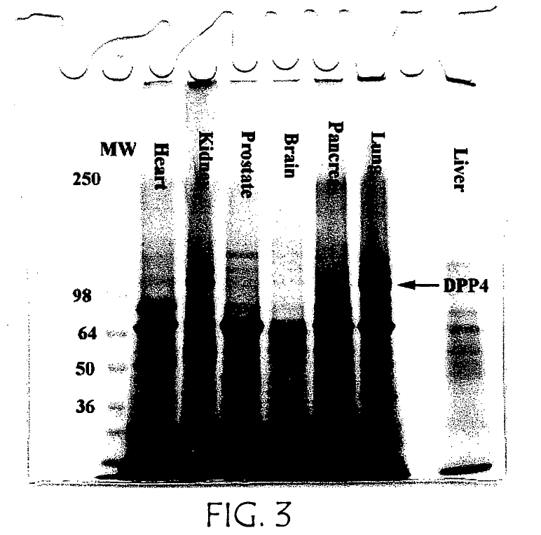

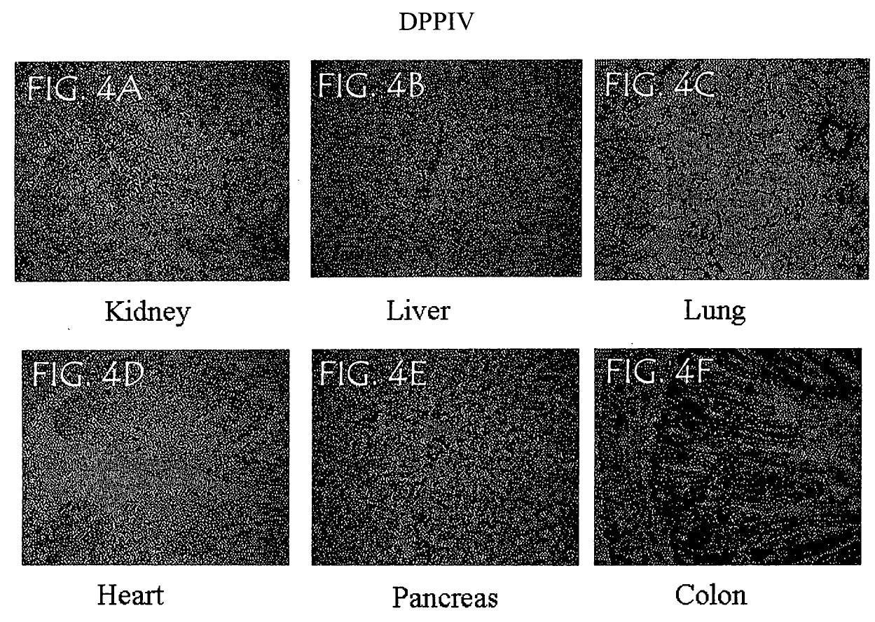

The luminal proteins of the vasculature of an entire rat were labeled with biotin. Then the organs were removed individually and the labeled proteins were isolated as described in Roben et al., U.S. patent Ser. No. 09/528,742, filed Mar. 20, 2000. The labeled proteins that were isolated from the homogenized lung were subjected to polyacrylamide gel electrophoresis and a protein (labeled DPP-4), which was specific to lung and kidney (FIG. 3), but predominately lung was identified. A peptide was sequenced corresponding to the sequence, FRPAE (SEQ ID NO: 37) and the protein was identified as rat liver dipeptidyl peptidase IV, Genbank Accession Number P14740 (nucleotide sequence Genbank Accession Number NM 012789). The full-length protein sequence corresponds to SEQ ID NO: 38 and the nucleotide sequence is SEQ ID NO: 39. The protein sequence is encoded by nucleotides 89-2392 of NM 012789. The human sequences correspond to SEQ ID NOS: 40 and 41. Genbank Accession Number NM 001935 is SEQ ID NO: 40 and the coding region of the mRNA is from nt 76 to 2376 (SEQ ID NO: 41). Previous studies suggest that the rat liver dipeptidyl peptidase IV has a membrane anchoring region consisting of its amino terminus. (Ogata et al., J. Biol Chem 264(6):3596-601 (1989)). A monoclonal antibody specific to rat dipeptidyl peptidase IV (BD Pharmingen, San Diego, Calif. Catalog number 22811) was injected into the tail vein of a rat (about 0.1 to 100 mg/ml). The tissue from various organs was treated using immunohistochemistry and the antibody to DPP-4 was shown to localize to lung and kidney (see FIG. 4). In FIG. 4 panel a. kidney, panel b. liver, panel c. lung, panel d. heart, panel e. pancreas, and panel f. colon.

An antibody to human DPP-4 is available for use in producing the therapeutic complex of the invention (BD Pharmingen, San Diego, Calif.).

### Example 3

### Identification and Sequencing of Carbonic Anhydrase IV

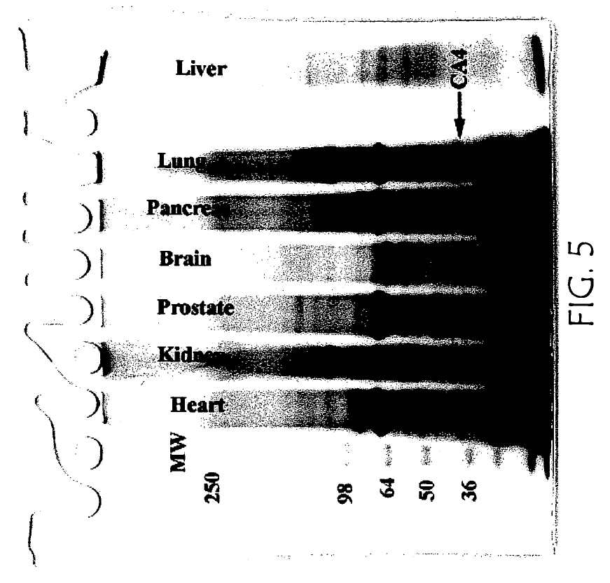

The luminal proteins of the vasculature of an entire rat were labeled with biotin. Then the organs were removed individually and the labeled proteins were isolated as described in Roben et al., U.S. application Ser. No. 09/528,742, filed Mar. 20, 2000. The labeled proteins that were isolated from the homogenized lung were subjected to polyacrylamide gel electrophoresis showed a protein (labeled CA-4), which was subsequently shown to be specific to lung and heart (FIG. 5). A peptide was sequenced corresponding to the sequence, DSHWCYEIQ (SEQ ID NO: 42) and identified as rat Carbonic Anhydrase IV, Genbank Accession Number NM-0 19174. The full-length protein sequence corresponds to SEQ ID NO: 43 and the nucleotide sequence is SEQ ID NO: 44. The human sequence corresponds to SEQ ID NOS: 45 and 46, Genbank Accession Number NM 000717. Previous studies suggest that carbonic anhydrase IV shows developmental regulation and cell-specific expression in the capillary endothelium (Fleming et al., Am J. Physiol, (1993) 265 (6 Pt 1):L627-35).

### Example 4

### Identification and Sequencing of Zymogen Granule 16 Protein (ZG16-p)

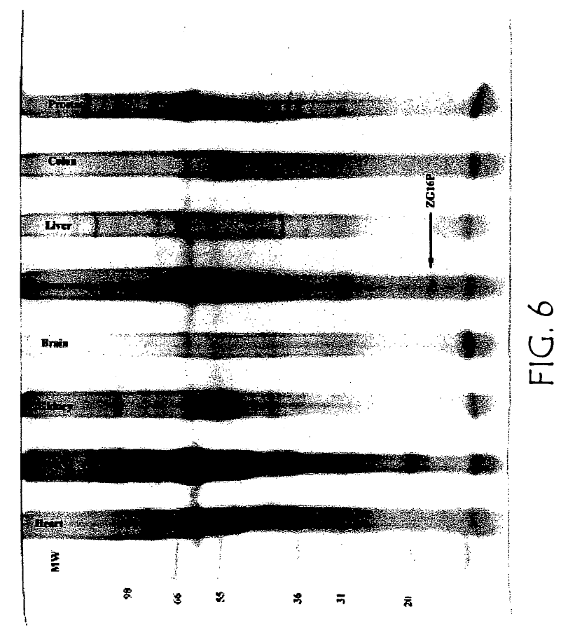

The luminal proteins of the vasculature of an entire rat were labeled with biotin. Then the organs were removed individually and the labeled proteins were isolated as described in Roben et al., U.S. application Ser. No. 09/528,742, filed Mar. 20, 2000. The labeled proteins that were isolated from the homogenized pancreas were subjected to polyacrylamide gel electrophoresis and a protein (labeled ZG16P) which was subsequently shown to be specific to pancreas and gut (see FIG. 6), but predominately pancreas was identified. The peptide was sequenced and the sequence NSIQSRSSSY, SEQ ID NO: 47 was obtained and identified as rat ZG16-p, Genbank Accession Number Z30584. The full-length protein sequence corresponds to SEQ ID NO: 48 and the nucleotide sequence is SEQ IDS NO: 49. The human sequence corresponds to SEQ ID NOS: 50 and 51, Genbank accession No. AF264625. Previous studies suggest that ZG16-p is located in zymogen granules of rat pancreas and goblet cells of the gut. (Cronshagen and Kern, Eur J. Cell Biology 65: 366-377, 1994).

### Example 5

### Identification and Sequencing of Rat MAdCAM

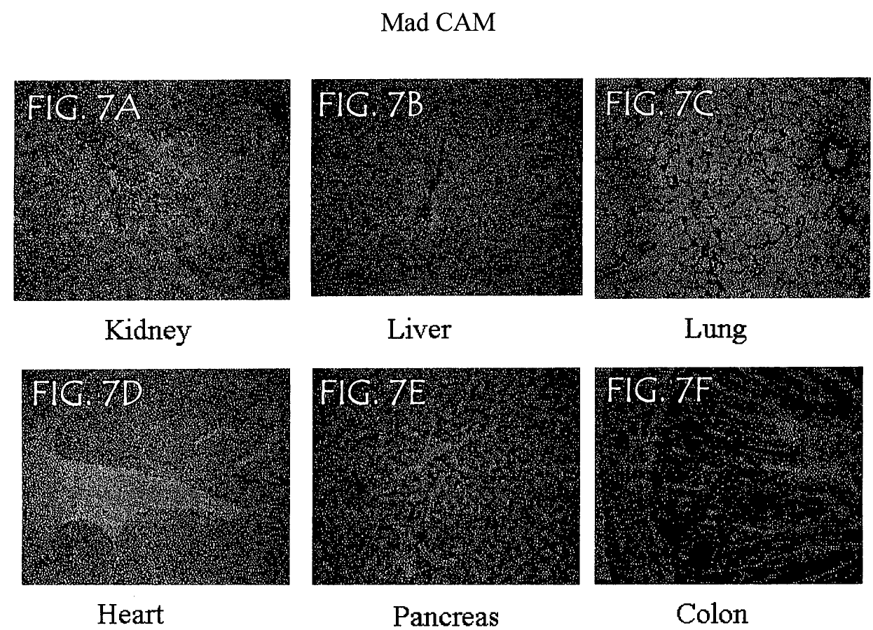

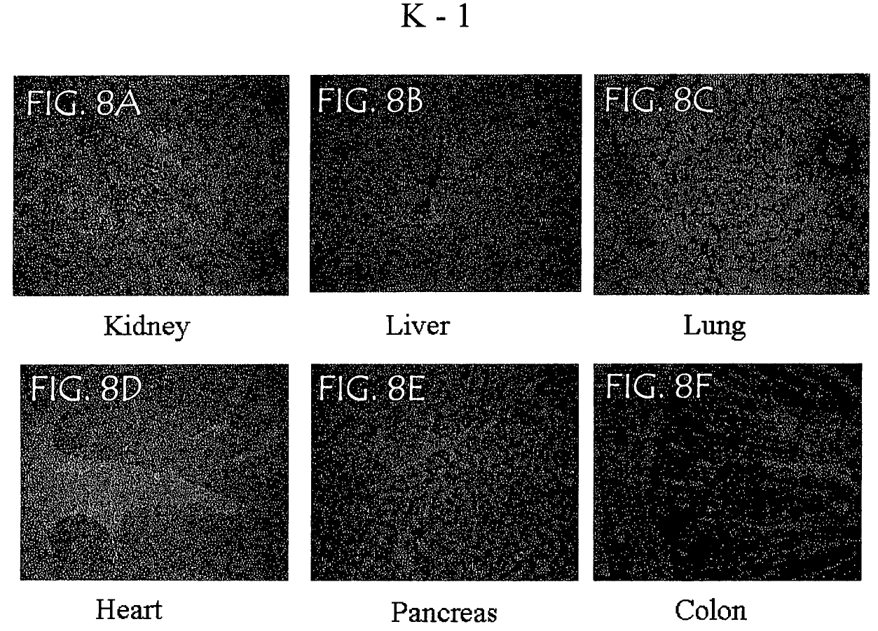

A monoclonal antibody was purchased from BD Pharmingen (catalog number 22861) and about 0.1 to 100 mg/ml were injected into the tail vein of a rat. The tissue from various organs was treated using immunohistochemistry and the antibody to MAdCAM (MadCam-1) was shown to localize to pancreas and colon (FIG. 7). In FIG. 7 panel a. kidney, panel b. liver, panel c. lung, panel d. heart, panel e. pancreas, and panel f. colon. Rat MadCam-1, Genbank Accession Number D87840 corresponds to protein sequence, SEQ ID NO: 52 and the nucleotide sequence is SEQ ID NO: 53. The human sequence corresponds to SEQ ID NOS: 54 and 55, Genbank. Accession Number U82483. A human MadCam-1 antibody is available from BD Pharmingen (San Diego, Calif.) to produce the therapeutic complex of the invention for human use.

### Example 6

### Identification of CD90

An antibody to the rat CD90 was purchased (BD Pharmingen, San Diego, Calif., catalog number 22211 D) and about 0.1 to 100 mg/ml was injected into the tail vein of a rat. The tissue from various organs was treated using immunohistochemistry and the antibody to Thy-1 was shown to localize to kidney (FIG. 8). In FIG. 8 panel a. kidney, panel b. liver, c. lung, d. heart, e. pancreas, and f, colon. Rat Thy-1, Genbank Accession Number NP036805 corresponds to protein sequence SEQ ID NO: 64 and Genbank Accession Number NM 012673 to nucleotide sequence SEQ ID NO:65. Human Thy-1, Genbank Accession Number XP006076 corresponds to protein sequence SEQ ID NO:66 and Genbank Accession Number XM 006076 to nucleotide sequence SEQ ID NO:67 (see also Genbank Accession Number AF 261093). A mouse anti-rat Thy-1 antibody is available from Pharmingen Intl. and was used for immunohistochemistry at a concentration of 0.5 to 5 μg/ml to produce the therapeutic complex of the preferred embodiment for human use.

### Example 7

### Identification and Sequencing of an Albumin Fragment

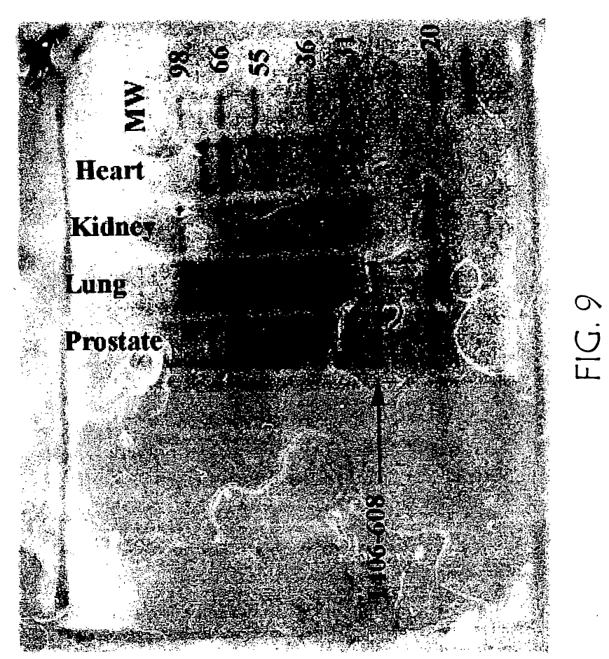

The luminal proteins of the vasculature of an entire rat were labeled with biotin. Then the organs were removed individually and the labeled proteins were isolated as described in Roben et al., U.S. application Ser. No. 09/528,742, filed Mar. 20, 2000. The labeled proteins that were isolated from the homogenized prostate were subjected to polyacrylamide gel electrophoresis which identified a protein labeled T436-608 (FIG. 9). The protein was partially sequenced and identified as a fragment of Albumin TQKAPQVST (SEQ ID NO: 56). In addition, sequencing showed that the prostate-specific form was a fragment in which translation was terminated early, corresponding to amino acids 436 to 608 of the full-length albumin protein (SEQ ID NO:57). The Albumin fragment has been identified by others as a vasoactive fragment (Histamine release induced by proteolytic digests of human serum albumin: Isolation and structure of an active peptide from pepsin treatment, Sugiyama K, Ogino T, Ogata K, Jpn J Pharmacol, 1989 Feb., 49(2): 165-71). The rat protein sequence is SEQ ID NO: 58 (Genbank Accession No. P02770). The human counterpart is shown as SEQ ID NO: 59, Genbank accession No. P02768.

In Example 8, the in vivo distribution of the luminally expressed target proteins isolated and identified in the previous Examples is described.

### Example 8

### Biodistribution of DPP-4, MadCam-1 CD90 and CA-4

The following example describes the use of specific labeled antibody ligands to visualize the biodistribution of several of the luminally expressed target proteins that were identified in previous Examples. Specifically, 50 μl of a 1 μg/μl solution of an antibody specific for DPP-4, MadCam-1, CD90 or CA-4 was injected into the tail veins of a group of Sprauge-Dawley rats. The antibody was allowed to circulate for about thirty minutes after which time the animals were sacrificed and their organs removed. Small cubes of brain, heart, lungs, liver, pancreas, colon and kidneys were excised, placed in embedding medium and immediately frozen. The frozen cubes were kept on dry ice until they were sectioned. The tissues were sectioned in 6 pm slices using a cryostat, air-dried overnight and fixed in acetone for two minutes. The fixed tissue sections were incubated with Cy3-labeled secondary antibodies, rinsed then mounted for subsequent image capture. At least three independent experiments were performed for each luminally expressed target protein.

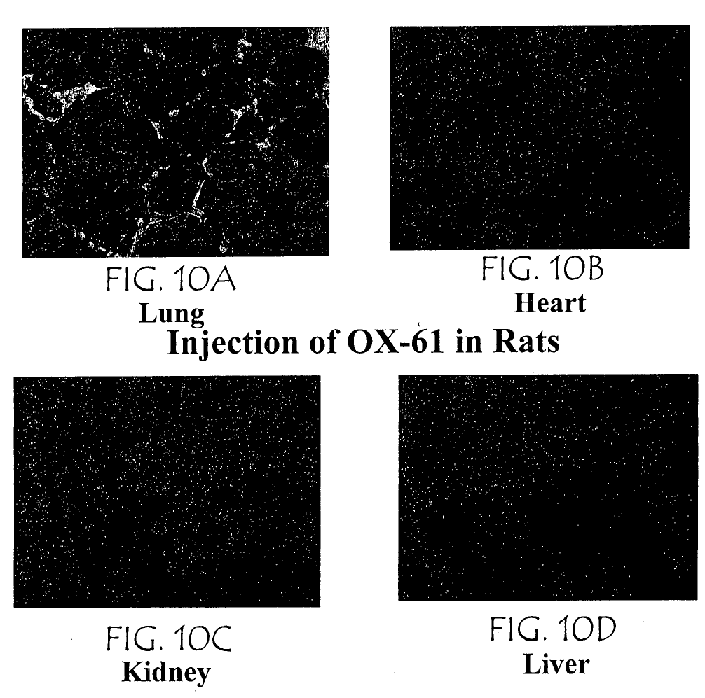

Using the above-described method, the biodistribution of DPP-4 was verified by using OX-61 (Pharmingen), a mouse monoclonal antibody that is specific for the luminally expressed target protein DPP-4. FIG. 10A shows strong fluorescent staining, which indicates that DPP-4 is present in the lung. Additional weak staining was observed in the glomeruli of the kidney (FIG. 10B); however, DPP-4 was not significantly found in any of the other tissues that were examined (FIGS. 10C-D). These results indicate that DPP-4 is primarily localized to the endothelium of the lung.

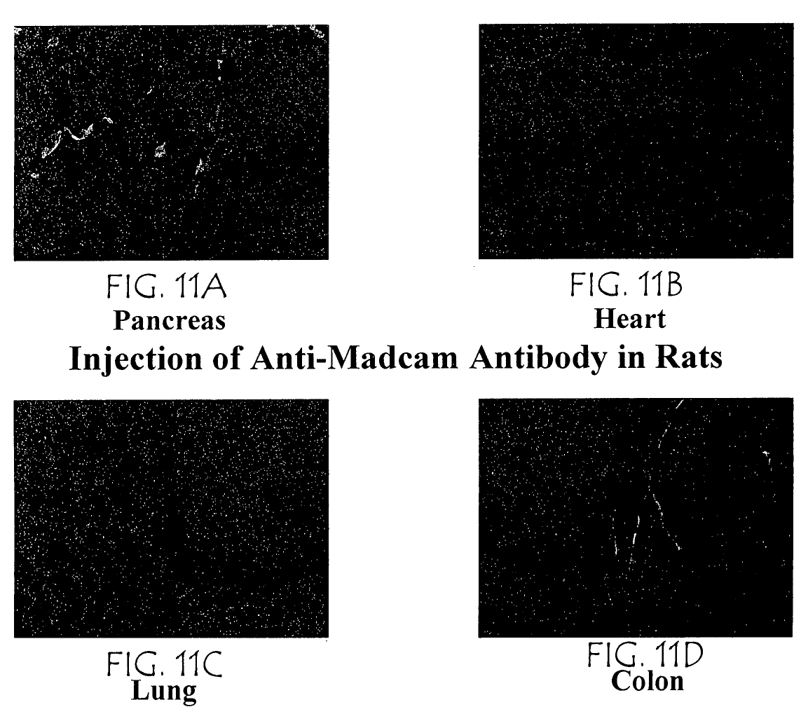

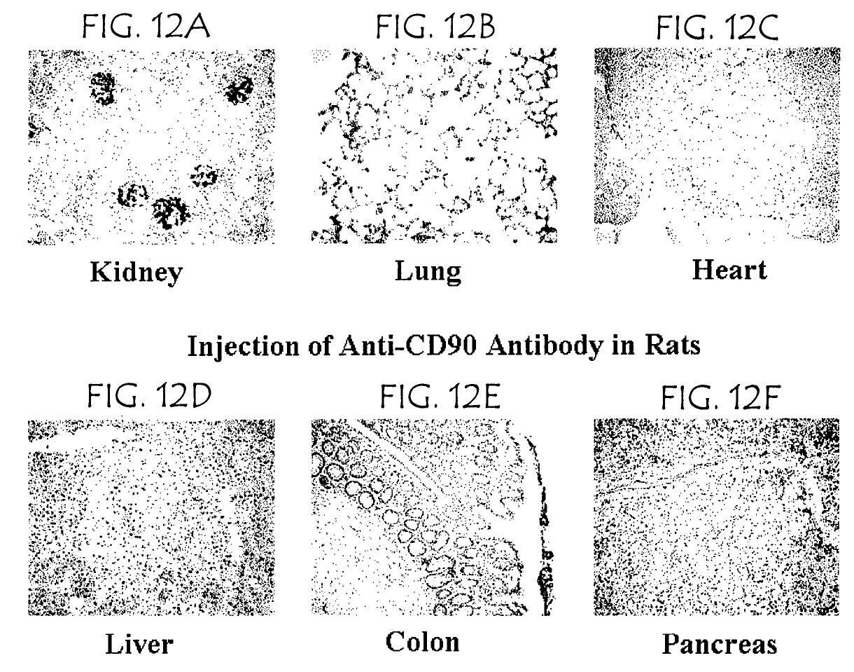

The biodistribution of MadCam-1 was also verified by using the above methods. Specifically, OST-2 (Pharmingen), a mouse monoclonal antibody that recognizes rat MadCam-1, was used. FIGS. 11A and 11D show that fluorescence was observed in both pancreas and the colon. Additional staining was observed in the small intestine. In contrast, very little fluorescence was observed in the other tissues that were examined (e.g. FIGS. 11B-C). These results indicate that MadCam-1 is localized to certain tissues that comprise the gastrointestinal (GI) tract.

The biodistribution of CD90 was verified by administering OX-7 (Pharmingen), a mouse monoclonal antibody that specifically recognizes rat CD90. FIG. 12A shows the fluorescent staining that was observed in the kidney. No staining was detected in any of the other tissues that were examined (FIGS. 12B-F). These results indicate that CD90 is localized only in the kidney.

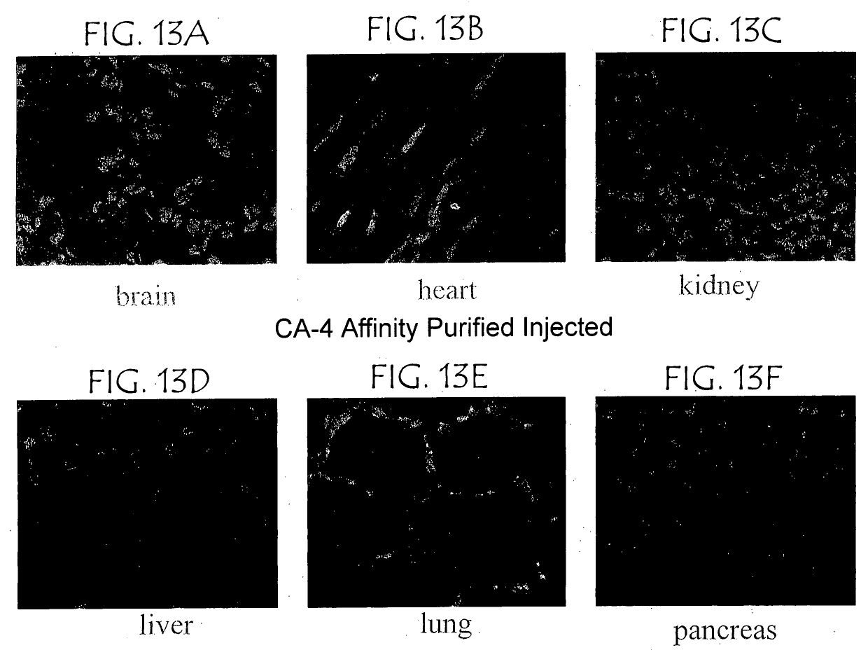

To determine the biodistribution of CA-4, a rabbit polyclonal antibody that recognizes rat CA-4 was generated using methods well known in the art. Using the above-described administration and histology procedures, this polyclonal antibody was then used to determine the localization of CA-4. Strong staining was observed in both the heart (FIG. 13B) and the lung (FIG. 13E) indicating the presence of CA-4. No staining was observed in brain (FIG. 13A), kidney (FIG. 13C), liver (FIG. 13D) or pancreas (FIG. 13F). A monoclonal antibody that is specific for CA-4 was also found to bind specifically to the heart and lung but not to other tissues. These results indicate that CA-4 is specifically localized to the heart and lung.

In Examples 9-13, the characteristics of ligand binding to specific luminally expressed proteins in target tissues is described.

### Example 9

### Relationship Between Ligand Dose and Specificity of Localization to Target Tissues

The following example describes the specificity of localization of antibody ligands to target tissues in relation to the amount of antibody that is administered. Specifically, mouse monoclonal antibodies specific to DPP-4, MadCam-1 or CD90 were administered to Sprague-Dawley rats via tail-vein injection. Each of the rats received either 5 μg, 20 pg, 50 μg or 100 μg of one of the above antibodies. Following the injection, the antibody was allowed to circulate for thirty minutes after which time the animals were sacrificed and their organs were removed. The organs were then processed for immunohistochemistry as described in Example 8.

Using the above-described method, the OX-61 monoclonal antibody was used to determine the relationship between the amount of antibody ligand administered and its specificity for the luminally expressed target protein DPP-4 in the lung. When administered to rats in doses of 5 to 50 μg, OX-61 displayed a high degree of specificity to the lung. However, when 100 μg or more was injected in a single dose, the OX-61 antibody began to appear in the kidneys. These results are consistent with the bioavailability data for DPP-4 presented in Example 8.

The monoclonal antibody, OST-2, was used in similar studies to determine the effect of dosage on its specificity for MadCam-1 in the pancreas and other GI organs. When administered in 5 μg, 20 pg, 50 μg and 100 μg doses, OST-2 remained specific for the pancreas and other tissues of the GI tract. These results seem to indicate that MadCam-1 specificity is limited to the GI tract irrespective of the dose that is administered.

The monoclonal antibody, OX-7, was used to determine the effect of dosage on its specificity for CD90 in the kidney. From doses of 5 to 50 μg, OX-7 displayed complete specificity for the kidney. However, at 100 μg, a small amount of OX-7 began to appear in the lung and liver. Although some OX-7 was detectable in lung and liver at high antibody concentrations, the amount of OX-7 present in the lung and liver was far less than the amount of OX-7, which appeared in the kidneys.

### Example 10

### Characterization of Ligand Binding to Target Tissues Over Time

The following example describes the binding of antibody ligands to specific target tissues throughout time. Specifically, mouse monoclonal antibodies specific to DPP-4, MadCam-1 or CD90 were administered to Sprague-Dawley rats via tail-vein injection. Each of the rats received a 50-μg dose of a single antibody, which was allowed to circulate for time periods ranging from 5 minutes to 48 hours. Following the period of antibody circulation, the animals were sacrificed and their organs were processed for immunohistochemistry as described in Example 8.

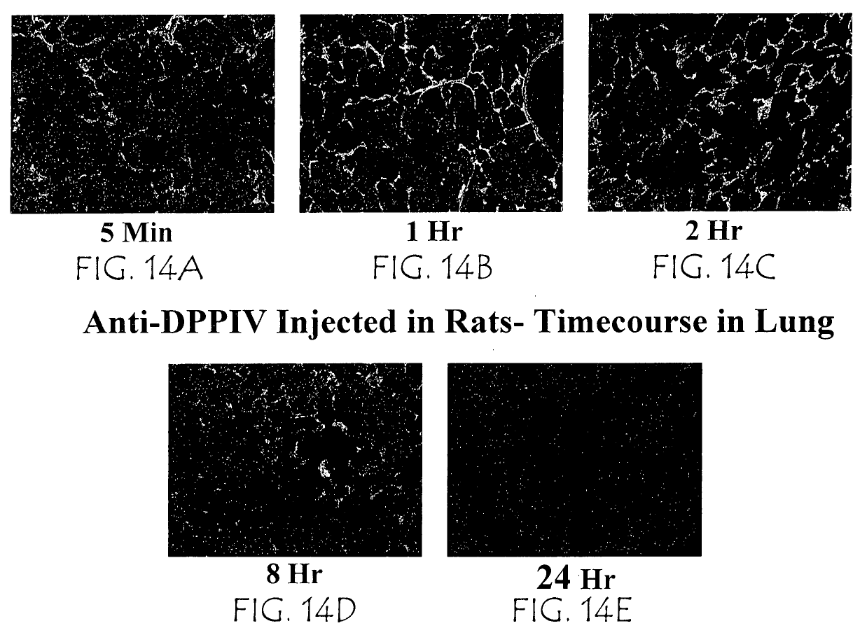

Using the above-described method, a profile of the binding of the OX-61 monoclonal antibody to DPP-4 in the vasculature of the lung was determined with respect to time. FIGS. 14A-E show the amount of OX-61 that localized to the lung during time periods ranging from 5 minutes to 24 hours after intravenous injection. Specifically, OX-61 was detected in the lung in as little as 5 minutes subsequent to administration (FIG. 14A). Similar amounts of this antibody were detected in the lung for at least eight hours after administration (FIG. 1413-D). At 24 hours subsequent to the administration, however, the amount of OX-61 detectable in the lung had significantly decreased (FIG. 14E).

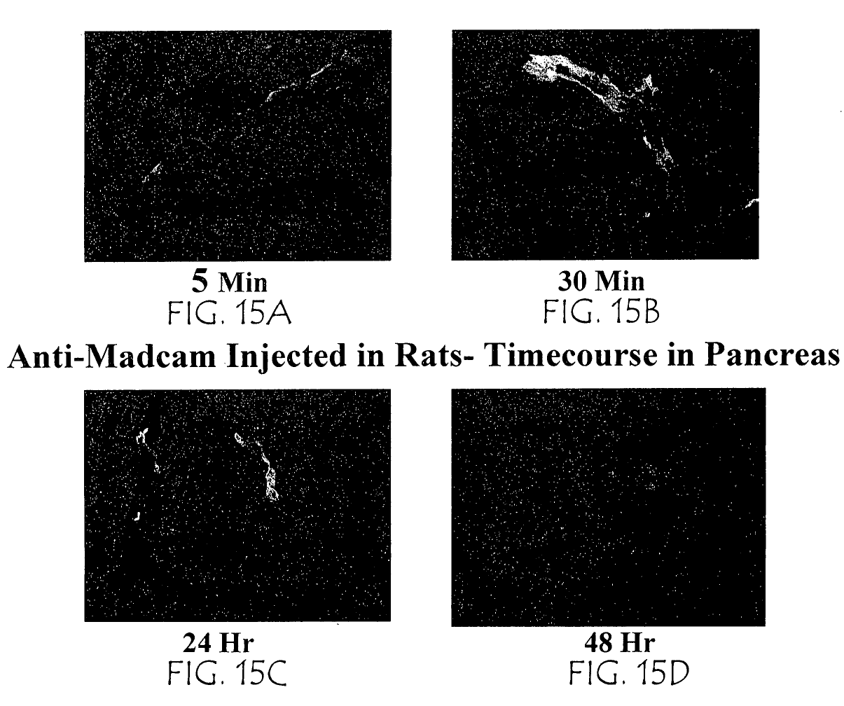

A profile with respect to time was established for the binding of the OST-2 monoclonal antibody to MadCam-1 in the vasculature of the pancreas. FIGS. 15A-D show the amount of OST-2 that was detected in the pancreas during time periods ranging from 5 minutes to 48 hours. Specifically, OST-2 was detected in the pancreas within 5 minutes subsequent to administration (FIG. 15A). In addition, similar amounts of this antibody were detected in the pancreas after 30 minutes, 24 hours and even 48 hours post injection (FIGS. 15A-D).

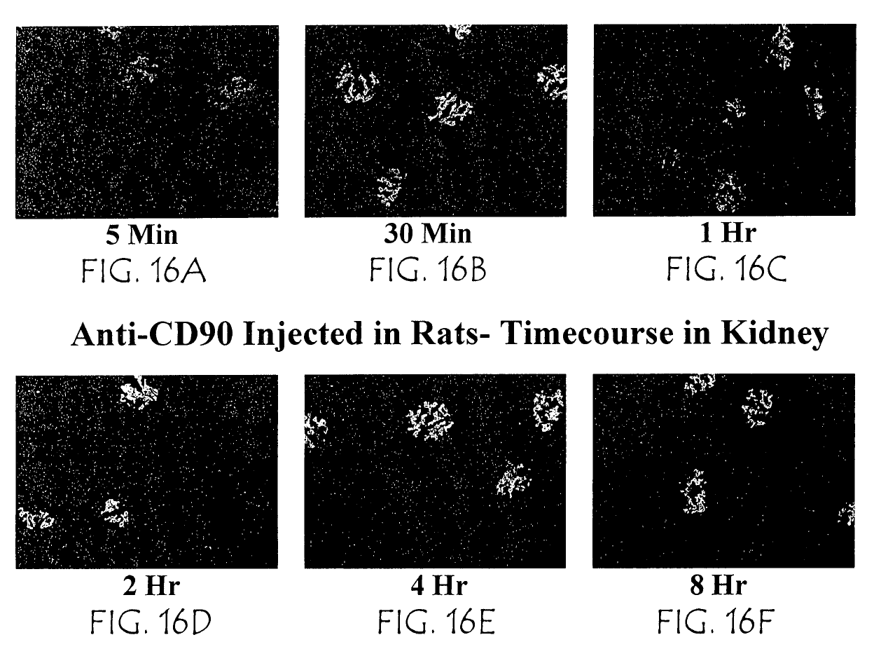

A profile with respect to time was also established for the binding of the OX-7 monoclonal antibody to the luminally expressed target protein CD90 in the vasculature of the kidney. FIGS. 16A-F show the amount of OX-7 that had localized to the kidney during time periods ranging from 5 minutes to 8 hours. Specifically, OX-7 was detected in the kidney in as little as 5 minutes subsequent to administration (FIG. 16A). Similar amounts of this antibody were detected in the kidney for at least eight hours after its administration (FIGS. 16B-F).

### Example 11

### Quantification of Antibody Ligand Bound to Target Tissues by Time-Resolved Fluorescence

The following example describes quantitative analyses of antibody ligands localized to luminally expressed target proteins in various target tissues. Specifically, antibodies specific for DPP-4, MadCam-1 or CA-4 were each labeled with approximately three molecules of Europium per antibody molecule using a europium-DTPA labeling kit (Perkin Elmer, Cat# AD0021) according to manufacturer's instructions. Additionally, monoclonal antibodies specific for influenza virus (IgG2a and IgG1 isotypes) were also labeled for use as isotype controls. After labeling, the antibody/Europium conjugates were injected into the tail veins of Sprauge-Dawley rats at doses of 5 μg, 20 μg and 50 μg. For each dosage level, the antibodies were allowed to circulate for either 30 minutes, 6 hours or 24 hours. At least three independent experiments were performed for each dose and time point combination.

At the end of each time period, the rats were sacrificed and their organs were processed for fluorescence analysis. Organs that were examined typically included, kidney, lung, liver, brain, pancreas, small intestine, large intestine (colon), stomach and heart. Excised organs were first homogenized in ten volumes of enhance solution (Perkin Elmer, Cat# 400-0010) then incubated overnight at 4° C. One percent of the resulting solution was then diluted 1:40 into fresh enhance solution, rotated for 30 minutes at room temperature and centrifuged at 1500 g for 10 minutes. The resulting solution was placed in a fluorimeter and the signal intensity was measured three times.

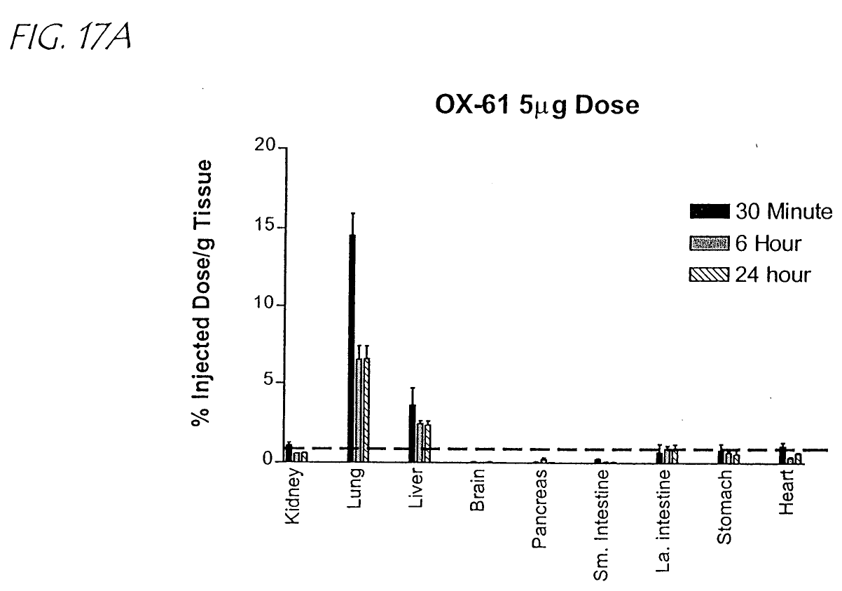

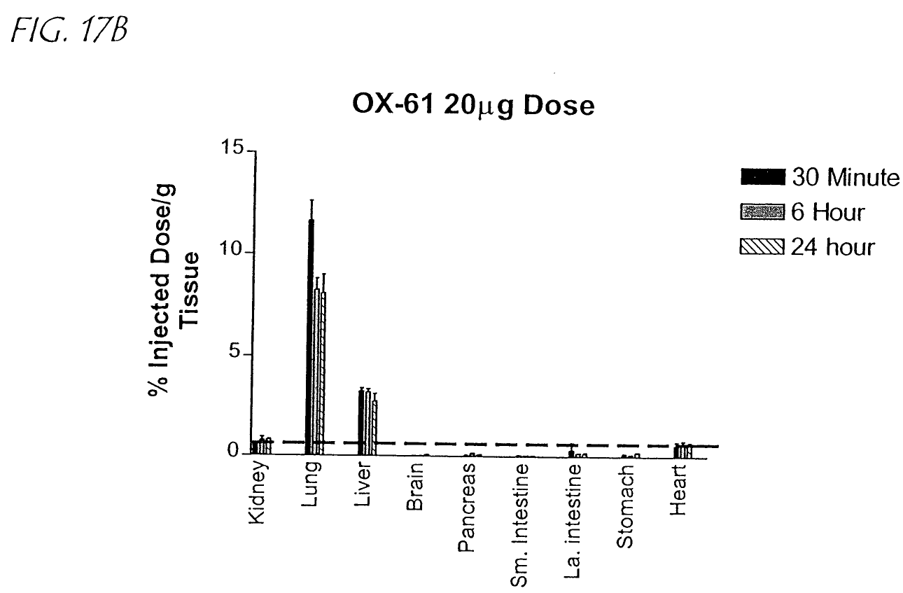

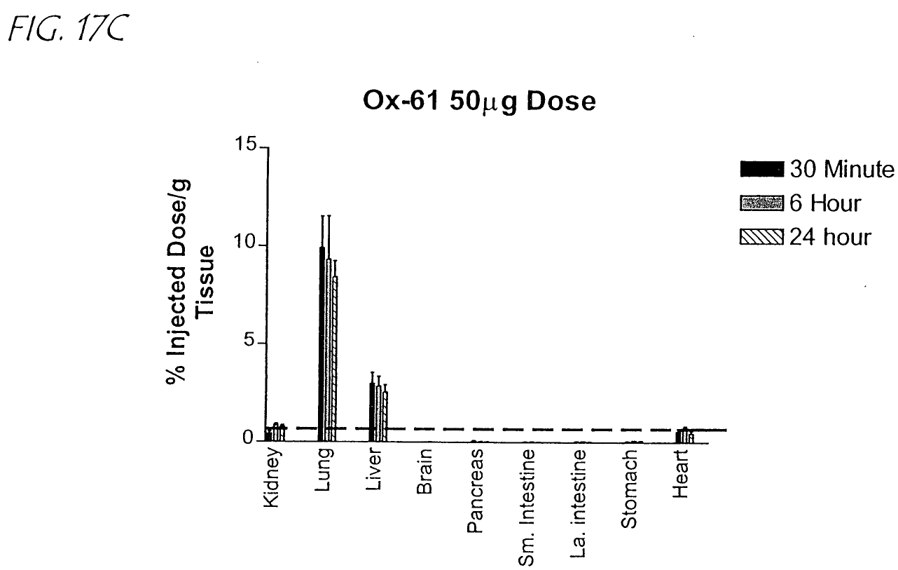

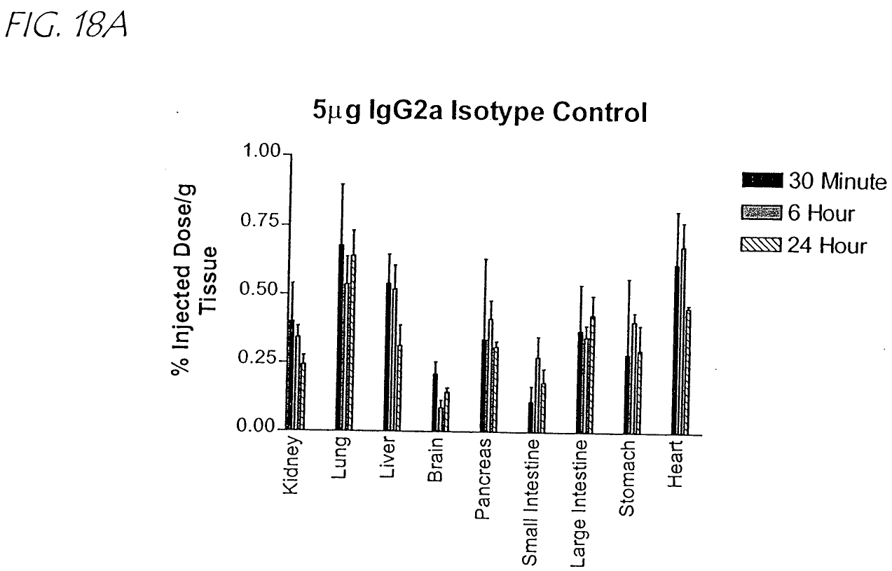

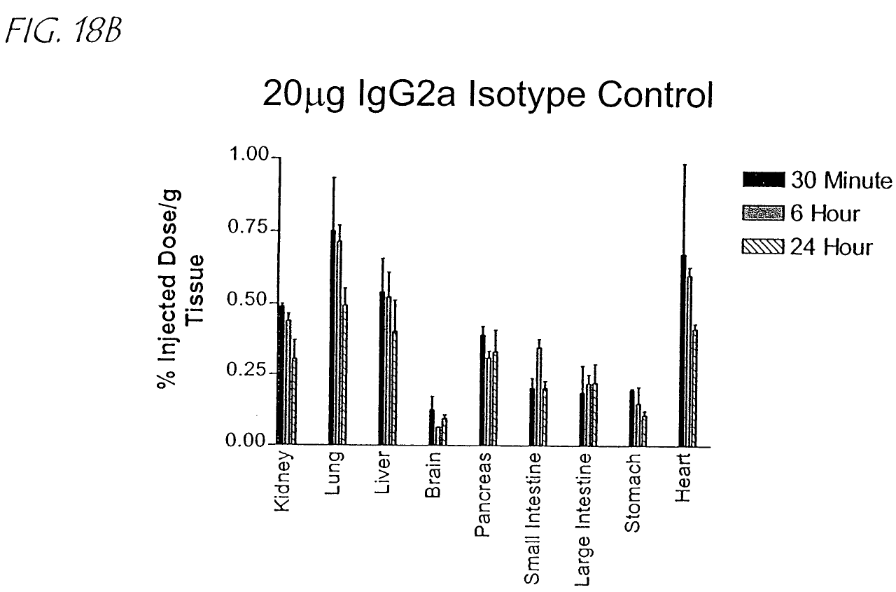

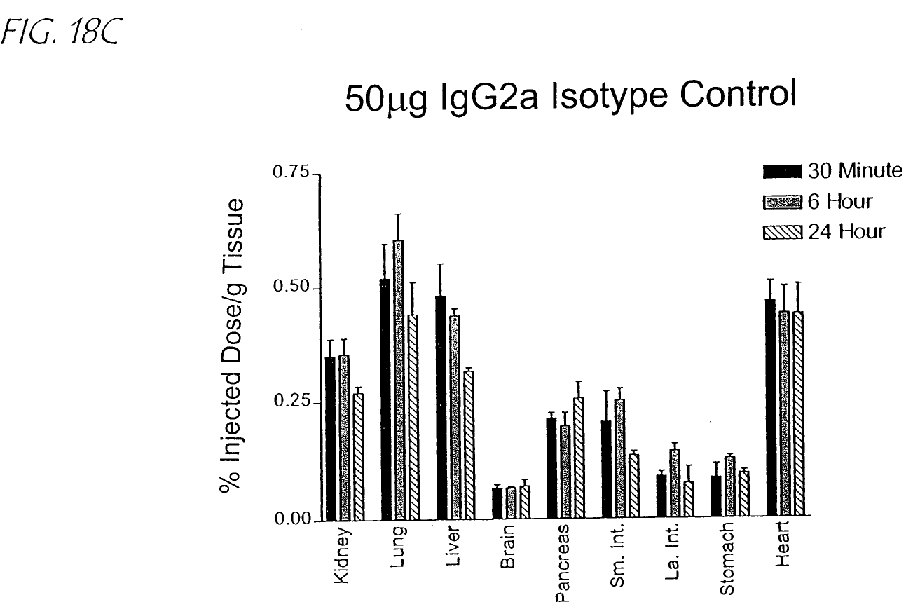

Using the above-described method, the amount of OX-61 (anti-DPP-4) antibody localized in each tissue type was determined at specific time points for each antibody dose that was administered. IgG2a isotype anti-influenza monoclonal antibodies were used as a control for background fluorescence. FIGS. 17A-C show the weight percent of OX-61 that was present in each tissue at each time point tested for each dosage level. Specifically, FIG. 17A shows that approximately 15% of the total 5 μg dose localized in the lungs after 30 minutes. By 6 hours, the level had fallen to about 7% but then remained constant up to the 24 hour timepoint. For the most part, the amount of OX-61 localized to other tissues was less than 0.75% of the dose weight, which corresponds to the maximum levels of anti-influenza control antibody that localized to each tissue type (FIGS. 18A-C and FIG. 17A, dashed line). One exception was the slightly increased localization to the liver.

Results similar to those obtained for the 5 NLg doses were also obtained for the 20 and 50 μg doses (FIGS. 17A-C, respectively). With respect to levels of OX-61 in the lung, it should be noted that as the initial dose increased, the percentage loss of OX-61 localized to the lung over time was reduced (FIGS. 17A-C). Taken together, these results indicate that high levels of OX-61 localize specifically to the lung and the levels of antibody remain high over a long period of time. Such high levels of localization will likely result in a significant improvement in the therapeutic index of any lung-acting drug delivered using this antibody ligand.

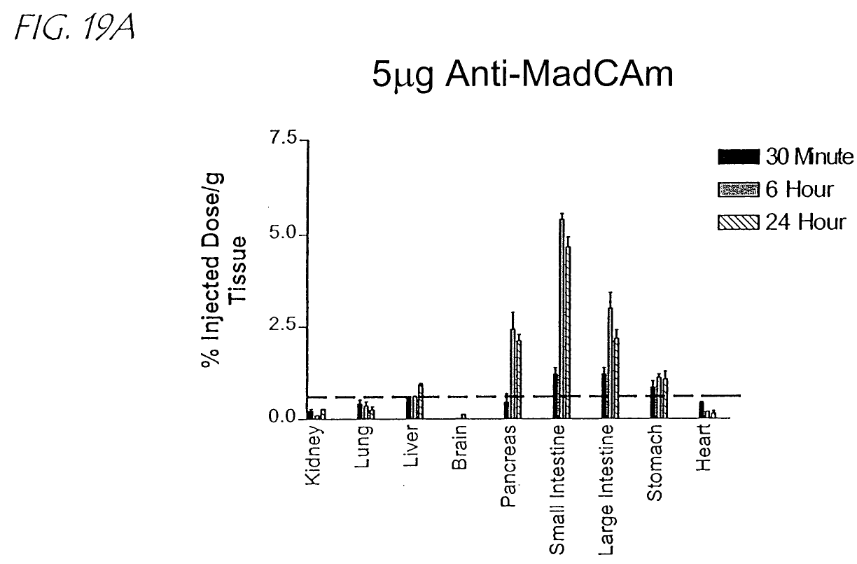

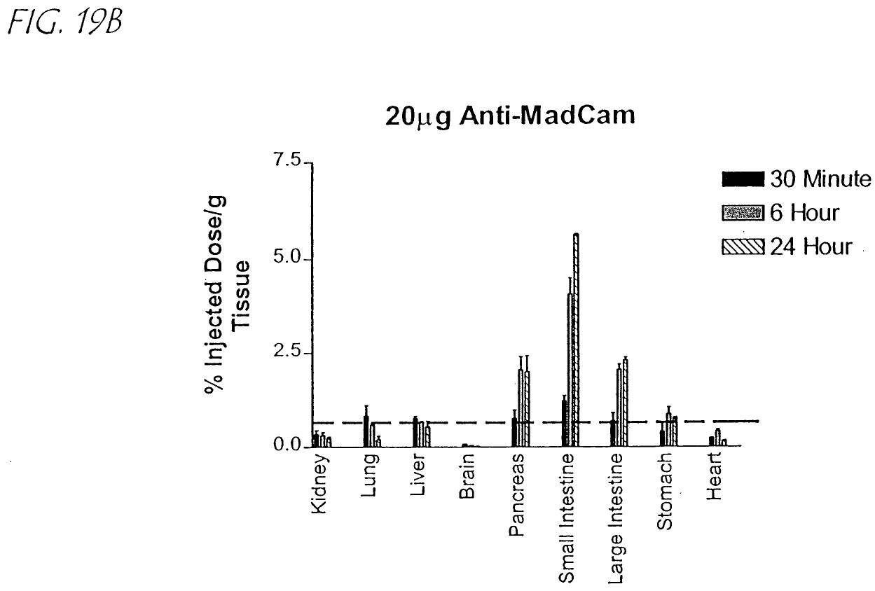

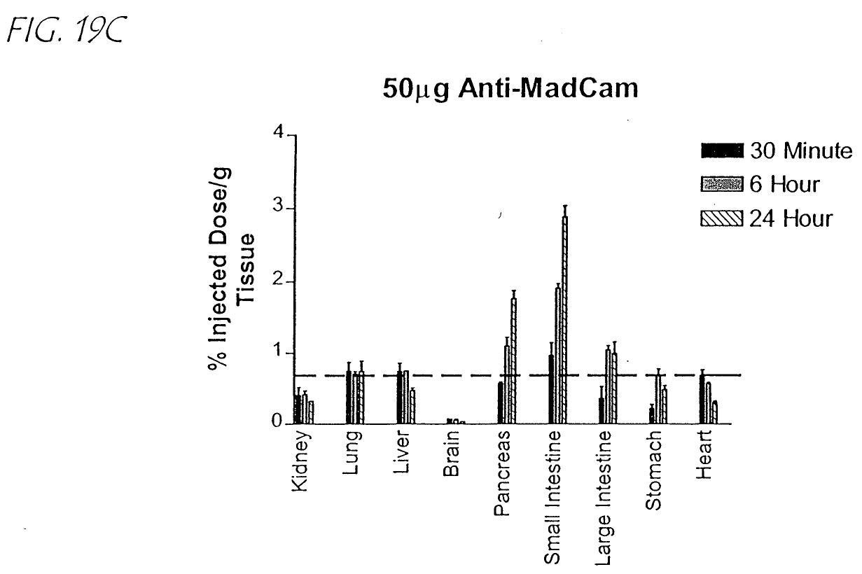

In additional experiments, the amount of OST-2 (anti-MadCam-1) antibody localized in each tissue type was determined at specific time points for each antibody dose that was administered. IgG1 isotype anti-influenza monoclonal antibodies were used as a control for background fluorescence. FIGS. 19A-C show the weight percent of OST-2 that was present in each tissue at each time point tested for each dosage level. Specifically, FIG. 19A shows that about 3% of the total 5-μg dose localized to the pancreas after 6 hours. Greater than 5% of the dose was observed in the small intestine after the same amount of time. The amount of OST-2 localized to non-GI tissues was generally less than 0.75% of the dose weight, which corresponds to the maximum levels of anti-influenza control antibody that localized to each tissue type (FIG. 19A, dashed line). It should be noted, that compared to the lungs, the pancreas is poorly vascularized. Accordingly, the percentage of antibody dose that is bound to this small area would be expected to be lower than for a antibody ligand that binds to a highly vascularized tissue such as the lung.

Results similar to those obtained for the 5 μg doses were also obtained for the 20 and 50 μg doses (FIGS. 19B and 19C, respectively). Additionally, the amounts of anti-influenza IgG1 isotype control antibody localized to each tissue was also similar to the amounts localized at the 5 μg dose level. There was at least one notable difference between the 5 μg dose and the two higher doses, however. At the 5 μg dosage, the amount of OST-2 localized in the GI organs peaked after 6 hours (FIG. 19A) and by 24 hours they began to fall. At higher doses, localization occurred in the pancreas and other GI organs cumulatively over the 24 hour time period. (FIGS. 19B-C). Taken together, these results indicate that high levels of OST-2 localize specifically to the GI organs, such as the pancreas, and the levels of this antibody increase over time. Such high levels of localization will likely result in a significant improvement in the therapeutic index of any drug delivered using this antibody ligand.

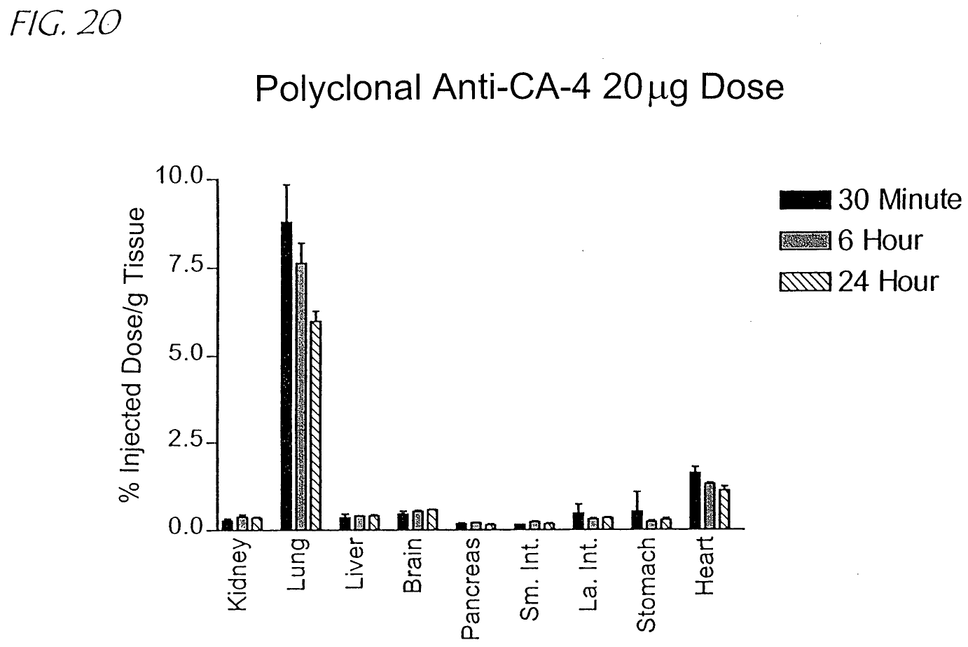

In similar experiments, 20 μg of Europium-labeled anti-CA-4 antibody ligand was administered intravenously to rats and the amount of ligand that localized in each tissue type was determined at specific time points. The affinity-purified rabbit polyclonal antibody to CA-4 (anti-CA-4), which was prepared as described in Example 8, was used as the tissue specific ligand. FIG. 20 shows that approximately 8.5% of the total injected antibody dose localized to the lung within the first 30 minutes. Approximately 2% of the antibody was found in the heart after the same time period. Levels of antibody in both the heart and lung slightly decreased after 6 hours then continued to decline when measured again at 24 hours. Anti-CA-4 did not accumulate significantly in any other tissues during the 24 hour timecourse.

### Example 12

### Quantification of Antibody Ligand Bound to Luminally Expressed Target Protein by Scintigraphy

The following example describes an alternative means for quantitatively analyzing antibody ligands localized to luminally expressed target proteins in various target tissues. OX-61 antibodies, which are specific for DPP-4, were radio-labeled with ¹²⁵I then either 1 μg or 5 μg doses were injected into the tail veins of Sprauge-Dawley rats and allowed to circulate for 5 minutes, 2 hours or 8 hours. Numerous tissues and fluids were analyzed by scintigraphic methods that are well known in the art. Results of the scintigraphy were expressed as nanogram equivalents of antibody per gram of tissue in each organ. The percentage of injected dose that localized to a particular organ was calculated using the known average weight of rat organs.

Using the above method, OX-61 was found to localize predominately to the lung. At both doses, OX-61 localized to the lung within the first five minutes. After two hours, 22% of the total injected 1 μg dose was found localized in this tissue. After 8 hours, the amount of antibody found in the lung increased to 30% of the injected dose. OX-61 was also found in the liver. Initially, a high level of OX-61 was observed in the liver; however, after 8 hours only 7% of the injected dose remained. Initial detection in the liver followed by the rapid decrease was most likely due to antibody circulating in the blood.

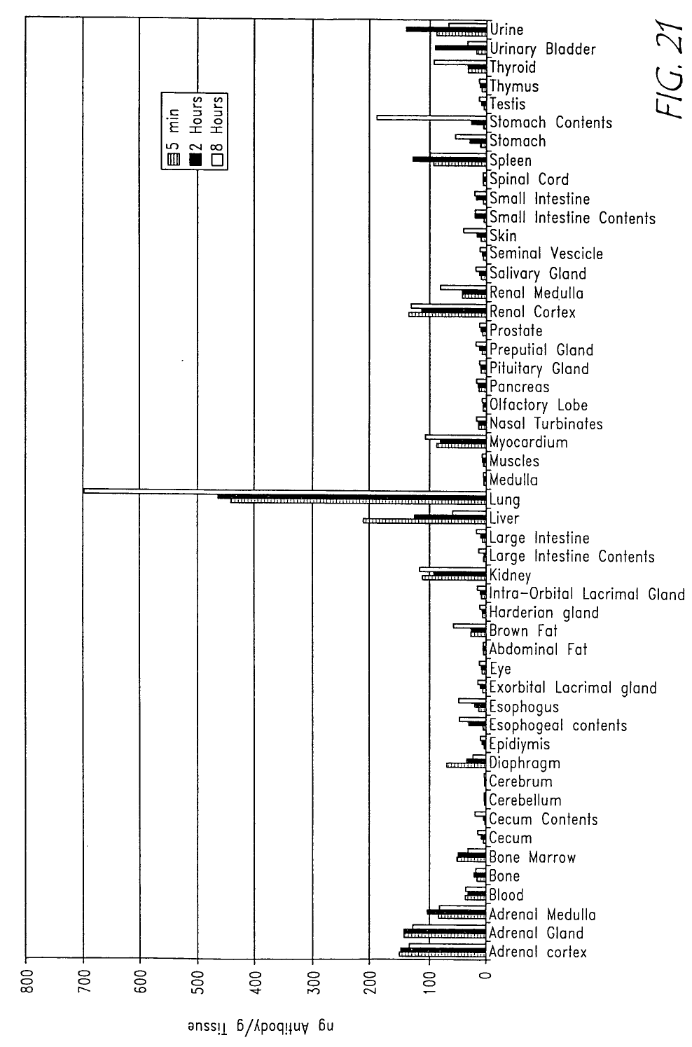

The results were similar when a 5 μg dose was administered. FIG. 21 shows that more than 0.4 μg of OX-61 per gram of tissue (20% of the initial antibody dose) localized to the lung after the first five minutes. After 8 hours, the amount of OX-61 increased to approximately 0.7 μg of OX-61 per gram of lung tissue. Throughout the timecourse, there was no significant build-up of OX-61 in any other tissue. These results confirm that high levels of OX-61 localize specifically to the lung and the levels of antibody remain high over a long period of time.

### Example 13

### Trancytosis of Antibody Ligands by Luminally Expressed Target Proteins

The following example describes methods that were used to characterize transcytotic, luminally expressed target proteins in terms of their ability to mediate transcytosis. More specifically, three-color histology was used to characterize luminally expressed target proteins capable of transporting bound ligand from the luminal surface of the blood vessel to the surrounding tissue space. Of the target proteins examined, only DPP-4 and CD90 appeared to have the ability to mediate transcytosis across the endothelial cell layer.

Three-color histology was performed using specific antibody ligands and stains specific for cellular structures. As in previous examples, antibodies specific to DPP-4, MadCam-1, CD90 or CA-4 were injected into the tail veins of Sprauge-Dawley rats in 50 pg doses. After 30 minutes, the rats were sacrificed and their organs were prepared for histology as previously described in Example 8. The tissue sections were then incubated with Cy3labeled secondary antibodies in order to detect bound primary antibodies. Additionally, the tissue sections were stained with 4′,6-diamidino-2-phenylindole, dihydrochloride (DAPI) and fluorescein-labeled Griffonia simplicifolia Lectin 1-isolectin B4 (GSL-1). DAPI stains the nuclei of the cells blue and GSL-1 stains the endothelium green. Transcytosis of antibody across the endothelium was detected by determining the distribution of yellow regions which were produced by the mixing of the red Cy-3 signal with the green-stained endothelium as antibody was transported across this cell layer.

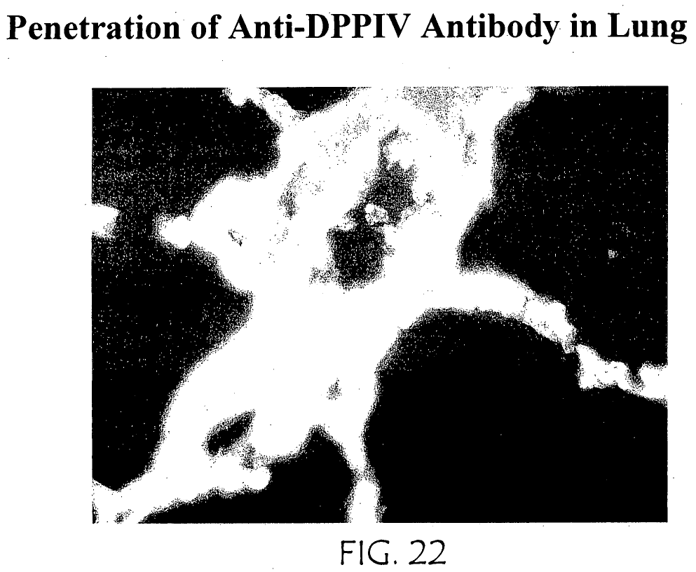

Using the above-described method, the transcytotic transport of OX-61 by DPP-4 was detected. FIG. 22 shows that OX-61 penetrated into the lung tissue surrounding the vasculature. As expected the surfaces of capillaries were stained green and cell nuclei were stained blue. Air-spaces in the lung were represented as black areas. The presence of yellow distributed throughout the endothelium indicated that the antibody was transported across the endothelial barrier and into the interstitial lung tissue.

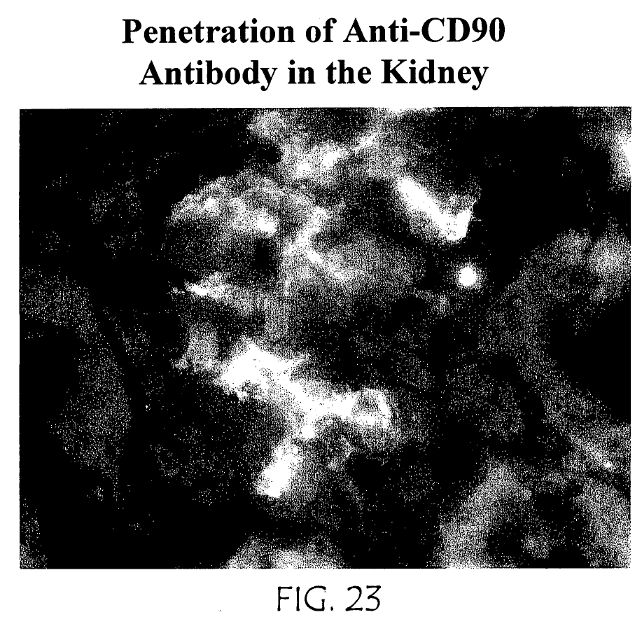

Similarly, the transcytotic transport of OX-7 by CD90 was detected. FIG. 23 shows that OX-7 penetrated into the glomerulus of the kidney. The penetration was indicated by the substantial amount of mixing that was observed between the bound antibody and the endothelium. This distribution of antibody into the endothelium can be seen in FIG. 23 as a diffuse area of yellow located between the red staining antibody that is bound at the luminal surface and the green staining endothelial layer.

Although OST-2 bound to MadCam-1 as expected, the antibody was not transported across the endothelium into the pancreas. FIG. 24 shows a section of the pancreas having no visible penetration of antibody into the endothelium. The antibody localized to the surface of the blood vessel (red) but never moved across the endothelium (green) and into the surrounding tissue. The absence of any yellow coloring in FIG. 24 demonstrates this lack of transcytosis.

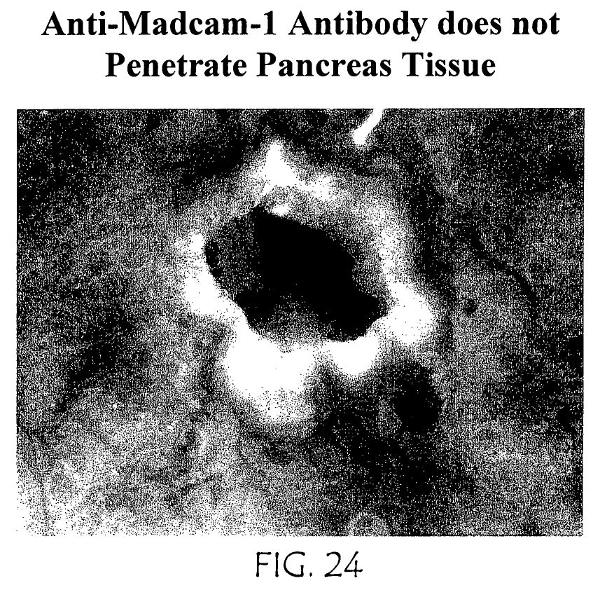

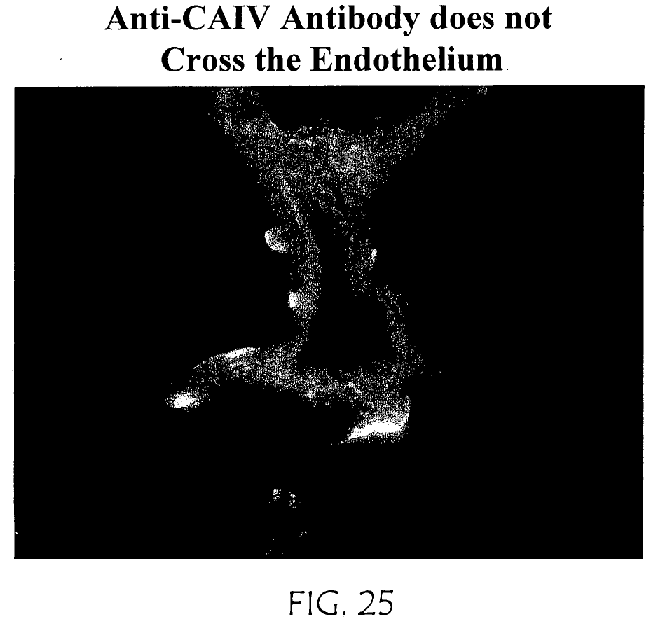

Similarly, no transcytosis was seen for anti-CA-4 antibody that was bound to CA-4 on the luminal surface of the vasculature of the lung. FIG. 25 shows a section of the lung having no visible penetration of antibody into the endothelium. In other words, the red areas of antibody bound to the endothelial surface never moved into the endothelial layer. This lack of movement is noted in FIG. 25 by the absence of yellow color intermixed in the endothelial cell layer. Similar results were noted for anti-CA-4 antibody that localized to the heart.

Taken together, the above results indicate that the luminally expressed target proteins that are identified herein are useful for both the delivery of drugs to the interstitium of specific tissues as well as their vascular surfaces.

Examples 14-16 describe therapeutic complexes comprising target-protein-specific antibody ligands that are linked to therapeutic moieties such as gentamicin and doxorubicin.

### Example 14

### Selective Drug Delivery to Tissues Using Specific Target Proteins

The following example describes the delivery of therapeutic complexes to specific target tissues. Therapeutic complexes were constructed by coupling mouse monoclonal antibodies specific to DPP-4 or MadCam-1 to either gentamicin or doxorubicin via a non-cleavable linker using methods well known in the art. On average, three molecules of drug were covalently conjugated to each antibody. Approximately, 50 μg of each therapeutic complex was administered to rats by tail vein injection and allowed to circulate for 30 minutes. The rats were then sacrificed and their organs were sectioned for histology using the method described in Example 8. Gentamicin and doxorubicin therapeutic complexes were detected by addition of either gentamicin- or doxorubicin-specific antibodies as appropriate, followed by signal amplification with Cy3 conjugated secondary antibodies. In some experiments, the tissue sections were also stained with 4′,6-diamidino-2phenylindole, dihydrochloride (DAPI) and fluorescein-labeled Griffonia simplicifolia Lectin 1-isolectin B4 (GSL-1) to demonstrate transcytosis (Three-color histology methods as described in Example 13).

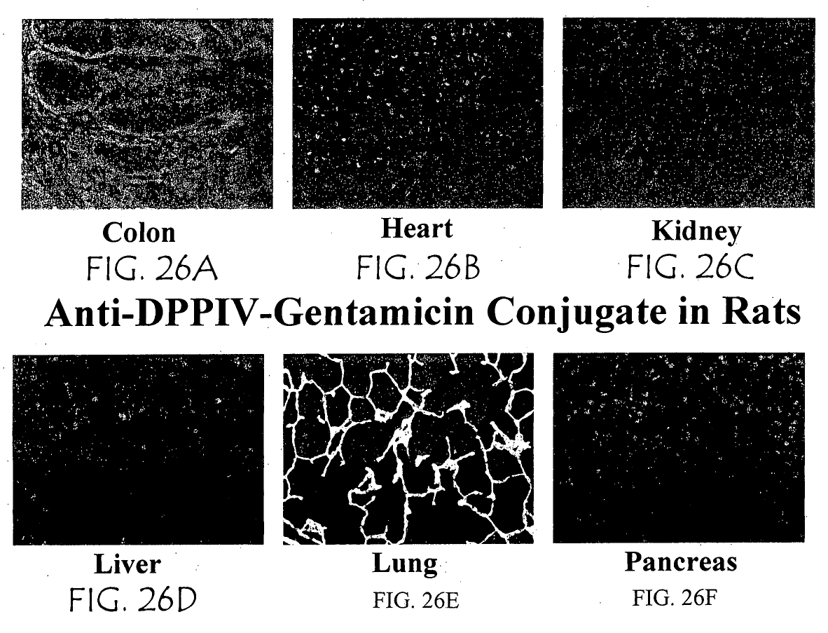

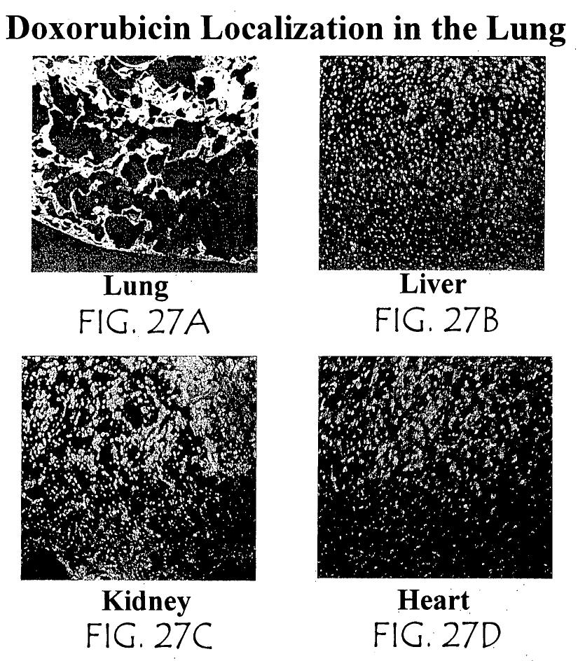

Using the above-described methods, OX-61/gentamicin and OX61/doxorubicin therapeutic complexes were found to localize specifically to the lung tissue within 30 minutes after the initial injection. FIGS. 26A-F shows the binding of the OX61/gentamicin therapeutic complex to specific tissues. Specifically, this therapeutic complex was observed in lung within thirty minutes following its injection (FIG. 26E). It was not present, however, in any other of the tissues examined (FIGS. 26A-D and 26F). Similar results were obtained for the OX-6 1/doxorubicin therapeutic complex (FIGS. 27A-D).

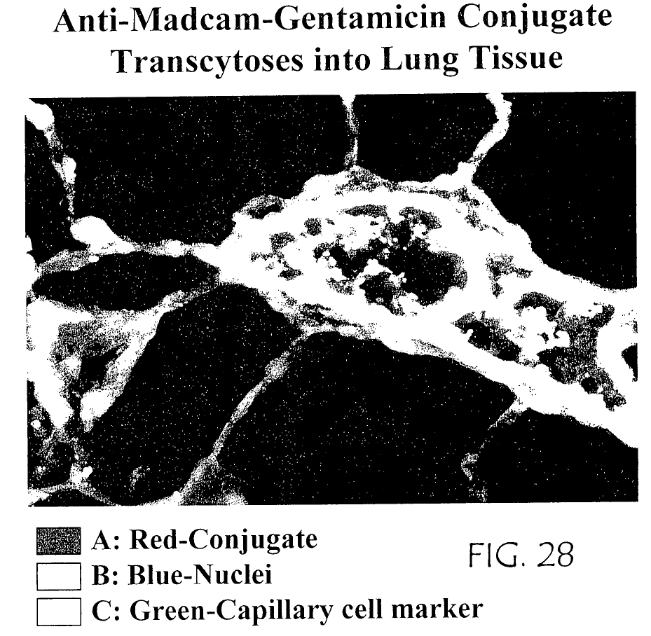

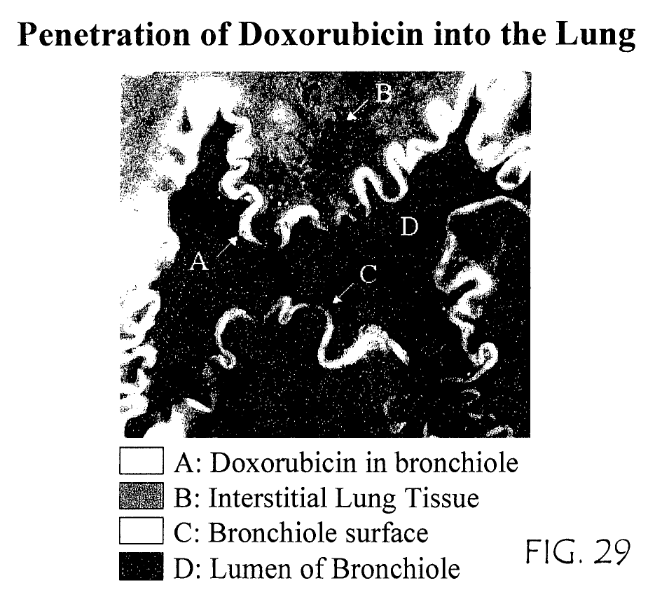

Using the above-described three color histology methods, DPP-4-mediated transcytotic transport of both OX-61/gentamicin and OX-61/doxorubicin therapeutic complexes was detected. FIG. 28 shows that the OX-61/gentamicin therapeutic complex penetrated the endothelium then localized into the interstitium of the lung. Therapeutic complexes were observed lining the capillaries and throughout the endothelial cell layer. Complexes were also observed throughout the interstitial tissues of the lung. The areas of yellow in FIG. 28 show the movement of the therapeutic complex across the endothelium. Similar results were seen for the OX-61/doxorubicin therapeutic complex. FIG. 29 specifically shows the accumulation of this therapeutic complex in the interstitium of the lung (FIG. 29, arrow B).

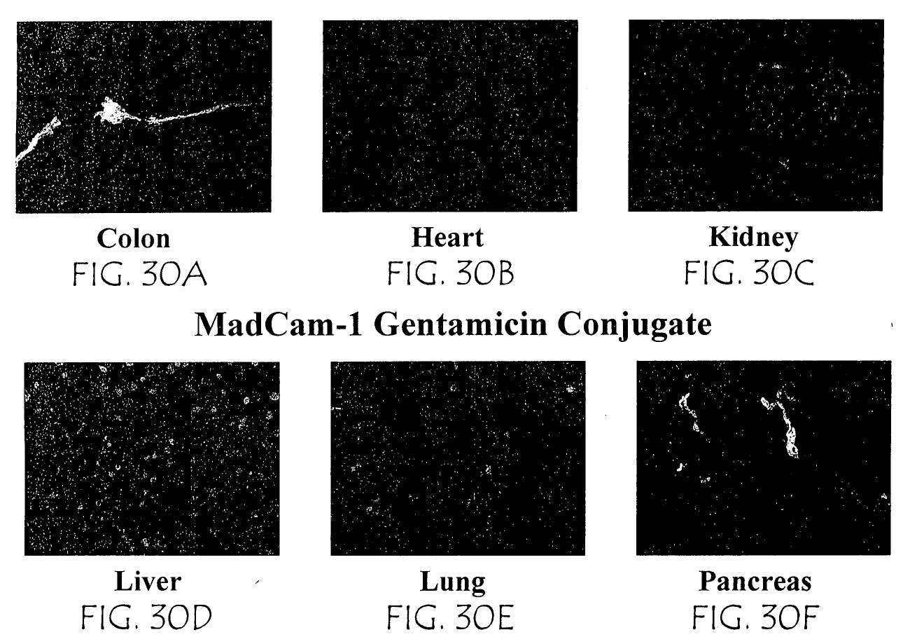

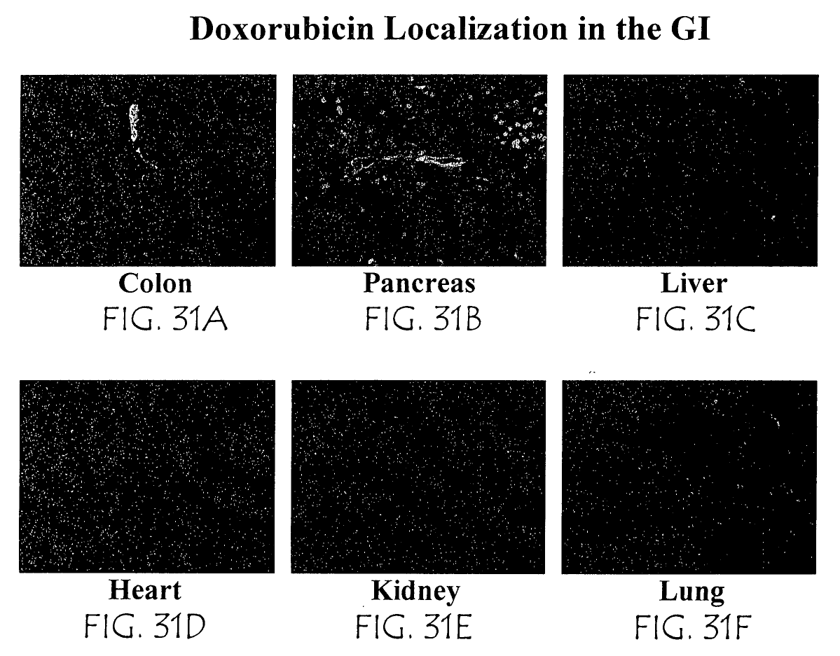

The tissue specific localization of ‘OST-2/genatmicin and OST2/doxorubicin conjugates was also evaluated. FIGS. 30A and 30F show that the OST2/gentamicin conjugate specifically bound to MadCam-1 in both the colon and the pancreas. This conjugate did not localize to any of the other tissues that were tested (FIGS. 30B-E). Similar results were observed for the OST-2/doxorubicin therapeutic complex (FIG. 31AF).

### Example 15

### Targeted Liposomal Formulations of Gentamicin Using the DPP-4-Specific Antibody OX-61

The following example describes the delivery of liposomal therapeutic complexes to specific target tissues. Therapeutic complexes were constructed by coupling mouse monoclonal antibodies specific to DPP-4 (ligand) to gentamicin (therapeutic moiety) using liposomes (linker). The liposomes were constructed using either egg phosphatidylcholine (EPC) or disteroylphosphatidylcholine (DSPC) as the main phospholipid component (greater than 50 mole percent). Maleimido-pegylated disteroylphosphatidylethanolamine (MPDSPE) was added as a minor lipid component in a concentration of about 5 mole percent. MPDSPE was synthesized by coupling polyethylene glycol (PEG) having a molecular weight of about 5000 kDa to disteroylphosphatidylethanolamine (DSPE). The free end of the attached PEG group was then converted to a reactive maleimide using methods well known in the art. The liposome formulation was completed by adding cholesterol in a concentration ranging 0 to 50 mole percent depending on the amount of phophospholipid that was initially used.

Therapeutic complexes were generated by coupling both gentamicin and OX-61 to the liposome linkers. Gentamicin sulfate was coupled by passively entrapping it within the liposomes during their formation. Gentamicin was entrapped at a concentration of approximately 150 μg/ml. Following the entrappment of the therapeutic moiety, the OX-61 antibody was coupled to the liposome linker. This coupling was accomplished by first reacting OX-61 with Traut's reagent to convert primary amines to thiols. The antibody was then coupled to the reactive MPDSPE.

The biodistribution of gentamicin administered in EPC and DSPC liposomes targeted to DPP-4 (EPC-DPP and DSPC-DPP therapeutic complexes, respectively) was compared to that of free gentamicin and gentamicin that was administered in untargeted liposomes. Specifically, a solution of free gentamicin or a dispersion containing therapeutic complexes or liposomes having no ligand bound to their surface was injected into the tail veins of Sprauge-Dawley rats at a dose of 150 pg gentamicin per rat. The rats were sacrificed after either 30 minutes or 18 hours and their organs were removed and homogenized. The amount of gentamicin in each organ homogenate was measured using a TDX analyzer (Abbott). At least three independent experiments were performed for each gentamicin formulation at each time point.

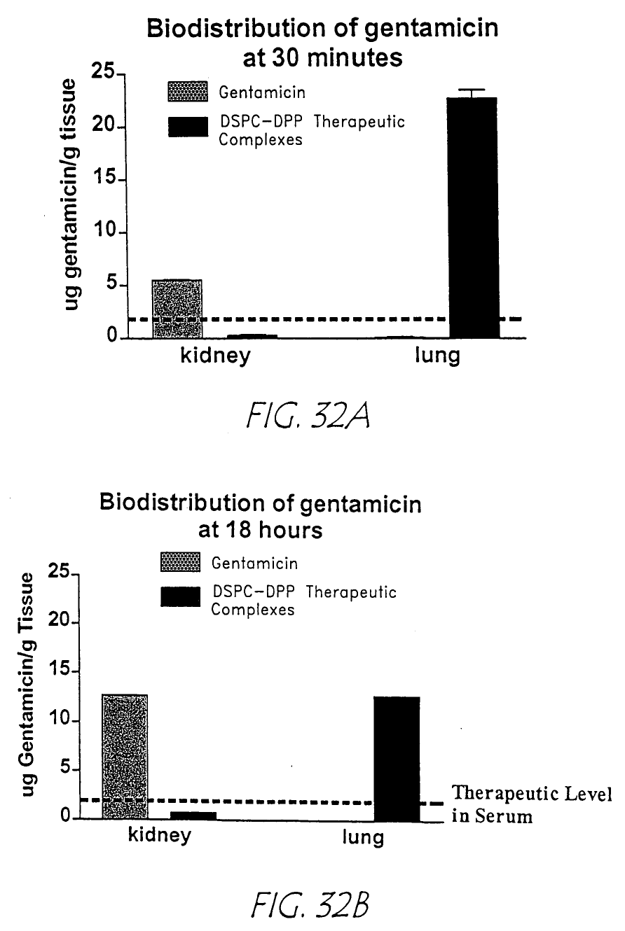

Using the above methods, the amount of gentamicin that localized to the lungs and kidneys after administration was determined for both free gentamicin and gentamicin administered in DSPC-DPP therapeutic complexes. In particular, within 30 minutes after administration, free gentamicin began to accumulate in the kidney (FIG. 32A). After 18 hours, the amount of gentamicin present in the kidneys more than doubled (FIG. 32B). In contrast, even after 18 hours, very little gentamicin appeared in the kidneys when administered in DSPC-DPP therapeutic complexes (FIGS. 32A-B). Nearly opposite effects were seen in lung tissue. FIGS. 32A-B show that, when administered in its free form, very little gentamicin was observed in the lungs either 30 minutes or 18 hours after injection. However, when administered in a DSPC-DPP therapeutic complex, gentamicin was present at about 20 μg per gram of lung tissue after 30 minutes (FIG. 32A). After 18 hours, the level fell by about half (FIG. 32B). These results indicated that build up of gentamicin in the kidneys, and thus gentamicin-mediated toxicity, can be prevented by delivering this drug specifically to the site of infection using appropriately targeted liposomal therapeutic complexes.

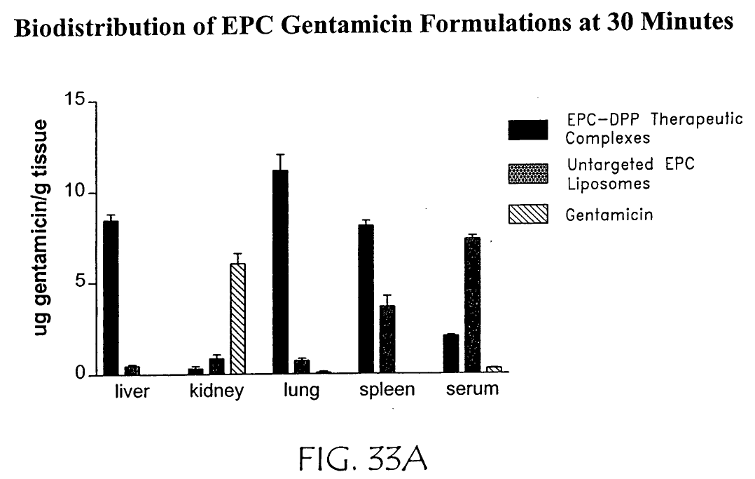

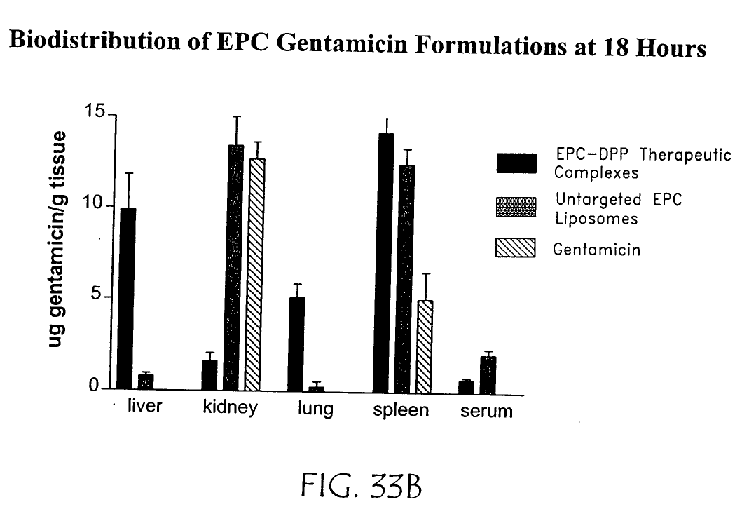

The biodistribution of free gentamicin was compared with that of gentamicin delivered in EPC-DPP therapeutic complexes and untargeted EPC liposomes. Within 30 minutes after administration of free gentamicin, a substantial amount of this compound appeared in the kidneys. After 18 hours, this amount more than doubled (FIGS. 33A-B). Gentamicin delivered in untargeted liposomes, appeared predominately in the serum after 30 minutes, but substantial amounts were detected in both the kidney and the spleen after 18 hours (FIGS. 33A-B). In contrast, within 30 minutes, most of the gentamicin delivered in EPC-DPP therapeutic complexes was distributed between the lung, liver and spleen but very little was observed in the kidneys or serum. The highest level of gentamicin, about 15% of the injected dose, was detected in the lung (FIG. 33A). Similar distributions were observed after 18 hours (FIG. 33B).

The above results indicate that gentamicin was targeted to lungs using EPC-DPP therapeutic complexes. Although the amount of gentamicin appearing in the liver and the spleen was significant, it is likely that the amount of drug accumulating in these organs can be reduced. Such a result can be achieved by using antibody fragments rather than whole antibodies as the targeting ligand. It has been well established that the Fc portion of antibodies mediate uptake into the liver and spleen. Accordingly, removing this portion of the antibody would likely reduce accumulation in these organs. Although accumulation of gentamicin in the kidney could not be prevented using untargeted liposomes, gentamicin could be effectively shielded from the kidney using the EPC-DPP therapeutic complex. Accordingly, such complexes are useful for both targeted drug delivery and preventing drug toxicity.

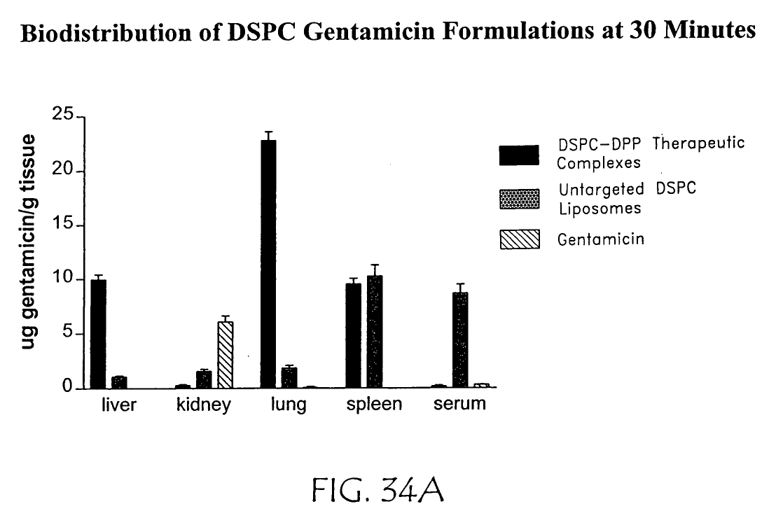

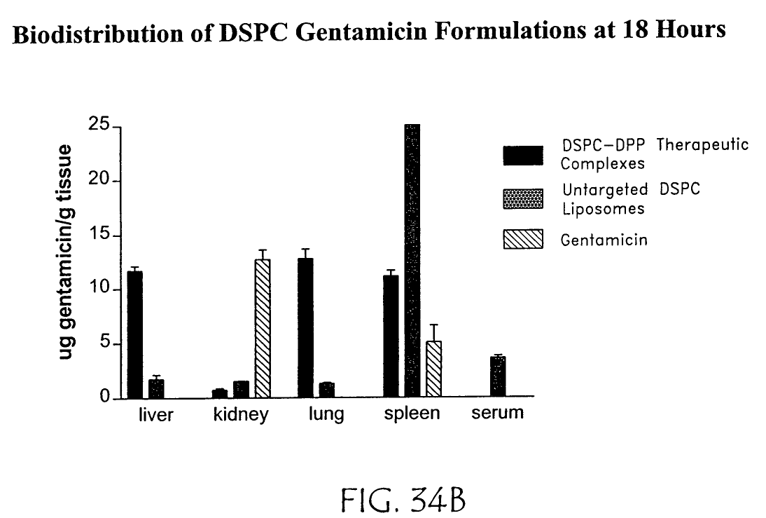

The biodistribution of free gentamicin was also compared with that of gentamicin delivered in DSPC-DPP therapeutic complexes and untargeted DSPC liposomes. FIGS. 34A-B show that the biodistribution of gentamicin delivered in DSPC-DDP therapeutic complexes both after 30 minutes and 18 hours was similar to that of gentamicin delivered in EPC-DPP therapeutic complexes with one significant difference. At both time points, DSPC-DPP therapeutic complexes localized over twice the amount of gentamicin in the lungs as EPC-DPP therapeutic complexes. (FIGS. 34A-B and 33A-B). The biodistribution of gentamicin delivered in untargeted DSPC liposomes was also similar to that of gentamicin delivered in untargeted EPC liposomes except far less gentamicin was found in the kidney after 18 hours when using DSPC liposomes for delivery (FIGS. 34A-B and 33A-B).

Taken together the above results indicate that DSPC-DPP therapeutic complexes were capable of targeting high levels of gentamicin to the lung. In addition, the use of such therapeutic complexes prevents the build up of gentamicin in the kidneys where it is known to have toxic effects.

### Example 16

### Efficacy of Therapeutic Complexes Containing Gentamicin

The following example describes the efficacy of EPC-DPP therapeutic complexes containing gentamicin in the treatment of pneumonia. Pneumonia was established in fifteen rats by infecting each animal with 1.5×10? Klebsiella pneumoniae via intratracheal injection. The rats were then divided into three groups having five animals each. After 24 hours, one group was treated by administering 5 mg/kg of free gentamicin per animal. A second group was treated by administering 5 mg/kg of gentamicin formulated in EPC-DPP therapeutic complexes per animal. The final group was left untreated as a control group. The rats were then monitored for survival over the next fifteen days.

The gentamicin delivered in EPC-DPP therapeutic complexes was superior to free gentamicin for the treatment of pneumonia. Only one of the five animals died in the EPC-DPP-treated group. This death occurred on day six. Each of the other four animals survived through day fifteen and displayed no signs of infection. Additionally, one of the surviving animals was sacrificed and no pathogenic bacteria were found in the lung. These results indicated that the gentamicin delivered in the EPC-DPP therapeutic complexes had completely cured the infection in 80% of the rats treated.

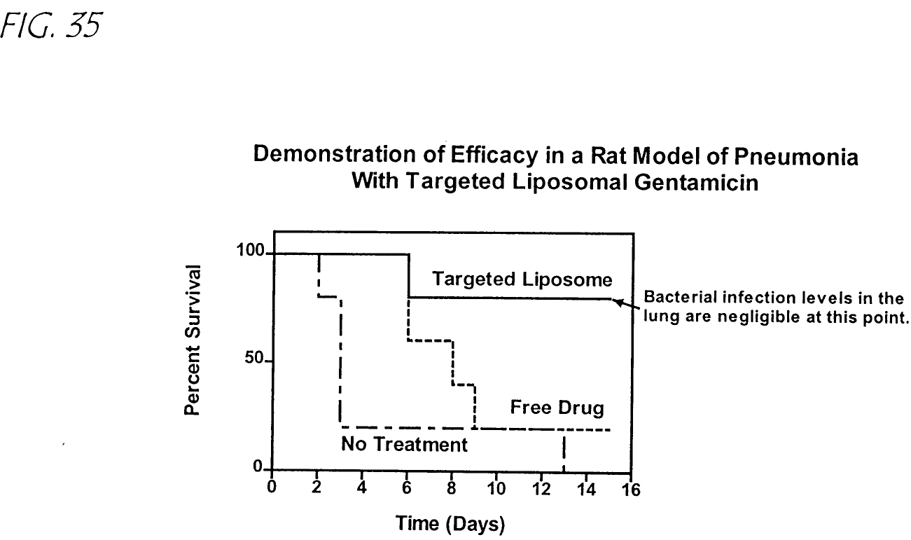

In contrast, all of the untreated rats died. Four of these animals died by day three. Four of the five animals treated with free gentamicin died by day nine. However, one animal did survive to day 15. Accordingly, the efficacy of free gentamicin was much less than that of gentamicin delivered to the lung in EPC-DPP therapeutic complexes (FIG. 35).

In Examples 17-22, the lung-specific luminally expressed molecule rat dipeptidyl peptidase IV (DPP-4) is used to produce a number of therapeutic complexes which are used to treat a variety of lung-specific diseases or deficiencies.

### Example 17

### Use of DPP-4 Doxorubicin Therapeutic Complex with an Acid Sensitive Linker for the Treatment of Lung Cancer

Initially, a therapeutic level of a human doxorubicin/DPP-4 complex such as that from Example 7 is administered to a patient intravenously. An effective amount of the complex is delivered to the patient, preferably 1 pg to 100 mg/Kg of patient weight in saline or an intravenously acceptable delivery vehicle. The DPP-4 F(ab′)₂ is specific for the lung tissue. As the therapeutic complex is transcytosed into the lung tissue, the acid sensitive linker is cleaved and the doxorubicin is free to intercalate into the DNA. Because the doxorubicin is incorporated into the DNA of cycling cells, the effect on the cancer cells which are in the process of cycling will be marked and the effect on the normal lung cancer cells much reduced. Therefore, the treatment results in a reduction of the number of cancer dells in the lung, with a minimum of side effects. Because doxorubicin generally targets dividing cells and, because of the tissue specificity, it will only affect the dividing cells of the lung, and therefore, it is envisioned that the number of cells killed due to side effects of the treatment will be minimal.

In Example 18 a method is set out for the synthesis and use of a DPP-4/doxocillin prodrug treatment for lung cancer.

### Example 18

### Use of DPP-4/Doxocillin Therapeutic Complex for the Treatment of Lung Cancer Using a Prodrug

The therapeutic complex is a DPP-4/β-lactamase conjugate which includes an F(ab′)₂ specific for DPP-4 linked to β-lactamase via a polypeptide linker, or a covalent bond. The linker used was SMCC. The chemotherapeutic agent doxocillin does not cross the endothelium due to a number of negative charges in the structure, which makes it nontoxic for all cells and ineffective as an anticancer drug. However, doxocillin can be thought of as a pro-drug which becomes active upon cleavage of the β-lactam ring to produce doxorubicin. Doxorubicin does cross the endothelium and intercalates into the DIVA of cycling cells, making it an effective chemotherapeutic agent.

Initially, a therapeutic amount of a DPP-4/β-lactamase complex is administered to the patient intravenously. The DPP-4 F(ab′)₂ is linked to the P-lactamase prodrug in the therapeutic complex using a linker which is not cleavable. The DPP-4F(ab′)₂ ligand is targeted to the lung tissue. A therapeutic level of the therapeutic complex is administered to the patient at between about 1 μg to 100 mg/Kg of patient weight. After administration and localization of the therapeutic complex, a therapeutic level of doxocillin is administered to the patient at between about 1 μg to 100 mg/Kg of patient weight, preferably between 10 μg to 10 mg/Kg of patient weight. The doxocillin is taken up systemically, but only in the microenvironment of the lung, the doxocillin is cleaved by the (3-lactamase to produce doxorubicin. Therefore, the eukaryotic cytotoxic activity of the prodrug is unmasked only at the location of the β-lactamase, that is, the lungs. The doxorubicin is taken up by the lung tissue and intercalates into the DNA. However, because the doxorubicin is incorporated into the DNA of cycling cells, the effect on the cancer cells which are in the process of cycling will be marked and the effect on the normal lung cancer cells much reduced. The treatment results in a reduction in the number of cancer cells in the lung.

In Example 19 a method is set out for the synthesis and use of a IDPP4/cephalexin prodrug therapeutic complex to treat pneumonia.

### Example 19

### Use of DPP-4 Therapeutic Complex for the Treatment of Lung Infections

The most common bacterial pneumonia is pneumococcal pneumonia caused by Streptococcus pneumoniae. Other bacterial pneumonias may be caused by Haemophilus influenzae, and various strains of mycoplasma. Pneumococcal pneumonia is generally treated with penicillin. However, penicillin-resistant strains are becoming more common.

The present invention is used for the treatment of pneumococcal pneumonia in humans (or other mammals) as follows: A therapeutic complex is constructed by linking the F(ab′)₂ fragment of human DPP-4 antibodies to cephalexin. The linker used is a liposome. The liposomes are constructed so that the F(ab′)₂ fragment is incorporated into the membrane and the cephalexin is carried within the liposome. Liposomes are produced by polymerizing the liposome in the presence of the DPP-4/F(ab′)₂ ligand such that the ligand becomes a part of the phospholipid bilayer and are prepared using the thin film hydration technique followed by a few freeze-thaw cycles. However, liposomal suspensions can also be prepared according to method known to those skilled in the art. 0.1 to 10 nmol of the therapeutic complex is injected intravenously. The liposomes carrying the cephalexin are targeted to the lung by the DPP-4 specific F(ab′)₂ fragments. Upon binding to the endothelium, the liposomes are taken up and the cephalexin is taken into the lung tissue. The cephalexin can then act on the cell walls of the dividing S. pneumonia organisms. One advantage of the targeting of antibiotics to a specific region is that less antibiotic is needed for the same result, there is less likelihood of side effects, and the likelihood of contributing to the drug resistance of the microorganism is considerably reduced.

In Example 20 a method is set out for the synthesis and use of a DPP-4/rifampin prodrug therapeutic complex to treat tuberculosis.

### Example 20

### Use of DPP-4 Therapeutic Complex for the Treatment of Tuberculosis

It can readily be envisioned that diseases such as tuberculosis, caused by the bacterium M. tuberculosis, which is often treated using rifampin or isoniazid for a very long period of time, would be more effectively treated using the therapeutic agent of the present invention. Much of the reason for the high incidence of disease and drug resistance in this microbe is the noncompliance with the extremely long course of treatment. It can be envisioned that using a method that directly targets the lungs with a high concentration of antibiotic would reduce the need for an unworkably long treatment and thus reduce the incidence of noncompliance and drug resistance.

The preferred embodiment is used for the treatment of tuberculosis in humans (or other mammals) as follows: A therapeutic complex is constructed by linking the F(ab′)₂ fragment of human DPP-4 antibodies to rifampin. The linker used is a liposome. The liposomes are constructed so that the F(ab′)₂ fragment is incorporated into the membrane and the rifampin is carried within the liposome. Liposomes are produced by polymerizing the liposome in the presence of the DPP-4/F(ab′)₂ ligand such that the ligand becomes a part of the phospholipid bilayer and are prepared using the thin film hydration technique followed by a few freeze-thaw cycles. However, liposomal suspensions can also be prepared according to method known to those skilled in the art. 0.1 to 10 nmol of the therapeutic complex is injected intravenously. The liposomes carrying the rifampin are targeted to the lung by the DPP-4 specific F(ab′)₂ fragments. Upon binding to the endothelium, the liposomes are taken up and the rifampin is taken into the lung tissue. The rifampin can then act on the M tuberculosis organisms.

In Example 21, a method is set out for the synthesis and use of a DPP-4/surfactant protein therapeutic complex to treat lung diseases resulting from underproduction of surfactant proteins.

### Example 21

### Use of DPP-4 Therapeutic Complex for the Treatment of Surfactant Deficiencies

A number of lung diseases, including emphysema, include, as part of the cause or effect of the disease, deficiencies of surfactant proteins. The present invention is used for the treatment of surfactant deficiencies as follows: A therapeutic complex is constructed by linking the F(ab′)₂ fragment of DPP-4 antibodies to a surfactant protein such as SP-A (surfactant protein A). The linker used is a pH sensitive bond. The therapeutic complex is injected intravenously into a patient's veins and is targeted to the lung by the DPP-4 specific F(ab′)₂ fragments. Upon binding to the endothelium, the therapeutic complex is transcytosed by the lung tissue and the change in pH cleaves the bond, thus releasing the surfactant protein.

In Example 22, a method is set out for the synthesis and use of a DPP-4/corticosteroid therapeutic complex to treat rejection of transplanted lung tissue.

### Example 22

### Use of DPP-4 Therapeutic Complex for the Treatment of Lung Transplantation Rejection

The present invention is used for the treatment of lung transplantation rejection as follows: a therapeutic complex is constructed by linking the F(ab′)₂ fragment of DPP-4 antibodies to an immunosuppressant such as a corticosteroid or cyclosporin with a pH sensitive linker. The therapeutic complex is injected intravenously into a patient's veins and is targeted to the lung by the DPP-4 specific F(ab′)₂ fragments. Upon binding to the endothelium, the therapeutic complex is transcytosed or taken up by the lung tissue and the change in pH cleaves the bond, thus releasing the immunosuppressant only in the area of the lungs. It can readily be seen that the advantage of such a treatment is that the patient is not immunosuppressed and still has a healthy active immune system during recovery from the surgery. The lung (or other transplanted organ) is the only organ which is immunosuppressed and is carefully monitored.

### Example 23

### Isolation of Molecules Exposed on Luminal Surfaces

The following example describes the methods used to selectively isolate molecules expressed on the luminal surface of vascular endothelial cells. Such methods have been described in detail in U.S. patent application Ser. No. 09/528,742, filed on Mar. 20, 2000. In particular, this example demonstrates the selective isolation of polypeptides present on the cell surface of vascular endothelium from various tissues of rats and pigs. Such organs include tissues of the brain, lung, heart and pancreas.

In some experiments, male Fisher rats were used. Each rat was anesthetized by injection with 1.6 ml of ketamine:xylazine mixture (7.5 mg/ml ketamine: 5 mg/ml xylazine). A tracheotomy was then performed by inserting a catheter into the trachea of the rat and attaching this to a bulb to provide ventilation. The thorax of the animal was then opened and pericardium removed. 0.5 ml heparin (2000 units/ml) was injected into each of the left and right ventricles. A 14-guage catheter was then attached to a perfusion line and inserted into the left ventricle and an incision was made to the right atrium to permit flow of the perfusion buffer. Although the amount of pressure was not critical, a range of between about 10 mm Hg and 80 mm Hg was typically used. In most experiments, perfusion was at 20 mm Hg.

To clear the vasculature of blood, a buffer of 60 ml Ringers at pH 7.5 with nitroprusside at 0.1 mg/ml was perfused. Second, the vasculature was prepared for reaction with the cell membrane impermeable reagent by perfusion with 60 ml of borate-buffered saline at pH 9.0. Third, about 20 ml of this same buffer containing the DTT cleavable reagent sulfosuccinimidyl-2-(biotinamido)ethyl-1,3-dithiopropioate (purchased as Sulfobiotin-X-NHS™ from Pierce Chemicals) was injected in the tissue and allowed to react for about one to two minutes. It will be appreciated that the time of reaction is not critical and may be varied significantly from the reaction time just described.

One of ordinary skill in the art will recognize that the amount of buffer that is used to deliver the cell membrane impermeable reagent is not critical provided that a sufficient amount is used to permit contact of the reagent with the vasculature of the tissues that will be examined. Additionally, the pH of the buffer is not critical. A range of between about 7.5 and about 9.5 can be used with this particular reagent. A skilled artisan will also recognize that the pH may be adjusted for use with other cell membrane impermeable reagents. It will also be appreciated that the concentration of the cell membrane impermeable reagent that is used may be varied. Concentrations of reagent from about 2 to about 50 mg/ml can also be successfully used to label luminally-exposed molecules.

After the reaction with reagent, 60 ml Ringers at pH 7.5 with 1.8 mg/ml glycine was perfused to remove excess biotin and to quench any remaining activated biotin. The pH of this quench buffer is not critical. A pH range of between about 7.5 and about 9.5 can be used. After this wash, 60 ml of 25 mM HEPES at pH 7.5 with 0.25 M sucrose and 10 mg/ml of various protease inhibitors, including leupeptin, pepstatin, E64 and PMSF, was introduced to prevent proteolysis. Organs and tissues were then separately removed and stored at −800C until ready for use.

It will be appreciated that the exact choice of protease inhibitors and their concentrations is not critical; however, a mixture which includes serine, cysteine, acid, metallo protease inhibitors is desirable.

Organ and tissue homogenization was carried out by mincing a known weight of tissue with a razor blade. The minced tissue was placed in ten volumes (v/w) PBS at pH 7.4, 1.0 mM EDTA, 1.8 mg/ml glycine with a cocktail of protease inhibitors, including AEBSF, leupeptin, pepstatin A, bestatin, aprotinin (Sigma Cat. # P8340), E64 and PMSF. The tissue suspension was homogenized in a dounce homogenizer with about ten to twelve up and down strokes at approximately 1500 rpm. The homogenate was then centrifuged in about 20 ml aliquots at 500×g for ten minutes in order to remove cell debris and nuclei. The supernatant was removed and placed in a fresh tube. Each pellet was washed with about ten ml homogenization buffer and the centrifugation was repeated. Supernatants were pooled and spun at 40,000×g for about two hours to pellet the membrane fractions. Each of these pellets was resuspended in about ten ml homogenization buffer and re-homogenized as before. SDS and Triton X-100 detergents were then added to a final concentration of about 1% each to solubilize the cell membranes and release proteins.

These solubilized membrane protein fractions were aliquoted into ten ml aliquots. Thirty ml of a 50% suspension of strepavidin beads (Pierce Chemicals) at 4 mg/ml binding capacity were added to each tube and this was inverted overnight at room temperature (RT). The beads were then allowed to settle into a pellet and the supernatant discarded. The pellet was washed five times with one ml homogenization buffer, 1% SDS, 1% Triton X-100 in order to remove non-specifically bound protein. Molecules modified with the biotin tag (i.e., the luminally-exposed vascular endothelial polypeptides bound to the membrane impermeable reagent) were specifically eluted from the beads by washing twice in mild conditions (i.e. 50 ml homogenization buffer with 50 mM DTT, 1% SDS, 1% Triton-X 100) Under these conditions, the DTT cleaved the internal disulfide domain of the membrane impermeable reagent, releasing the luminally-exposed vascular endothelial polypeptides and leaving the biotin bound to the immobilized streptavidin.

The eluted luminally-exposed vascular endothelial proteins were then precipitated with four volumes methanol, one volume chloroform and three volumes water, with mixing after each addition. The solution was centrifuged at 14,000 rpm for five minutes in a standard laboratory mircocentrifuge to separate the phases. The upper phase was removed and three volumes of methanol were added. The solution was centrifuged again to repellet the protein.

It will be appreciated that the general isolation procedures described herein for rats can be adapted for use with any animal. For example, the above method was used to isolate luminally-exposed polypeptides from pigs by increasing the volume of the buffers used for perfussion.

### Example 24

### Identification of Luminally-Exposed Molecules Expressed in a Tissue-Specific Manner

The following example describes methods used to determine the profile of luminally-exposed polypeptides that were isolated from tissue samples using the methods described in Example 23. These profiles were then compared to identify those luminally-exposed polypeptides that are expressed in a tissue-specific manner.

In pig, polypeptides expressed on the lunimal surface of vascular endothelial cells from brain, lung, heart and pancreas tissues were isolated using the methods described in Example 23. In preparation for polyacrylamide electrophoresis (PAGE), pellets of the isolated polypeptides were resuspended in sample buffer, which comprised 83 mM Tris HCl, pH 6.8, 1% 2-mercaptoethanol (2-ME), 2% SDS, 10% glycerol, then boiled for five minutes. After boiling, the samples were loaded onto a 4 to 20% gradient acrylamide gel (Novex) and subjected to electrophoresis for 1.5 hours at 150 volts. The resulting gels were stained with Gelcode Blue™ stain (Pierce Chemical) in order to visualize the polypeptide profile for each of the different tissues that were analyzed.

In some cases, samples of the isolated luminally-exposed polypepitdes obtained as described herein were subjected to two-dimensional electrophoresis to facilitate further isolation from similar sized polypeptides. Methods for performing two-dimensional gel electrophoresis are described in Rabilloud et al. Electrophoresis 18:307-319 (1997).

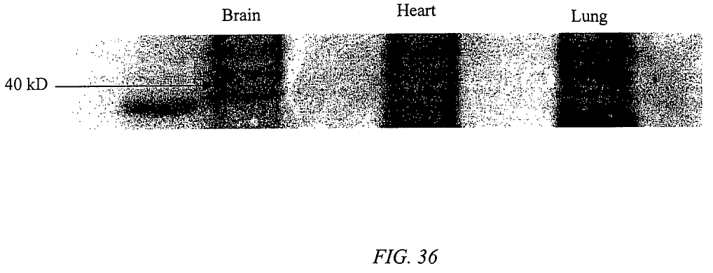

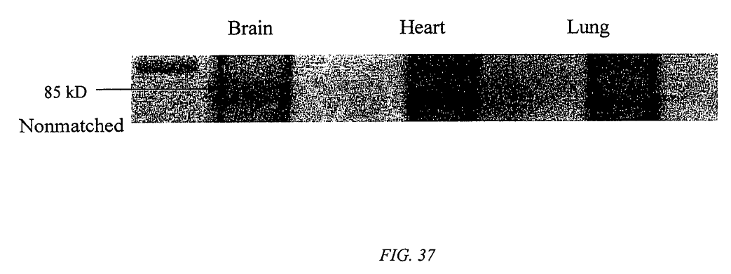

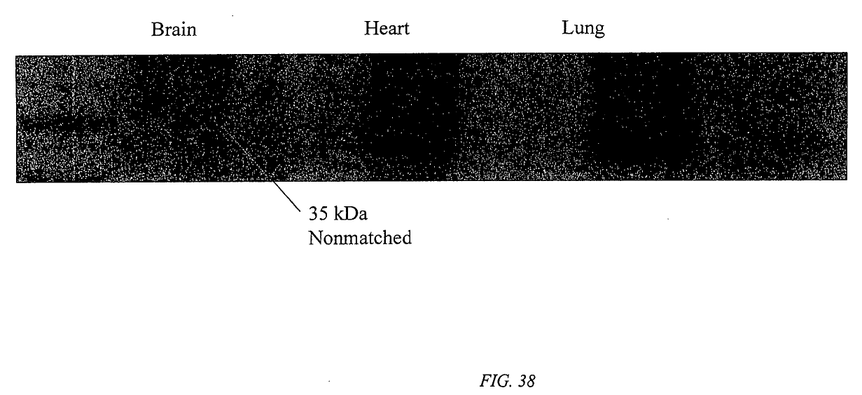

Pig brain was studied to identify any luminally-exposed polypeptides expressed solely or predominantly in cerebral tissues. FIG. 36 shows an approximately 40 kDa polypeptide that was found to be present in the sample of pig brain but was not found in the other tissues analyzed, such as heart or lung. Similarly, an approximately 85 kDa and an approximately 35 kDa polypeptide were found to be present in brain tissue but were not found in the other tissue types that were analyzed (see FIGS. 37 and 38, respectively).

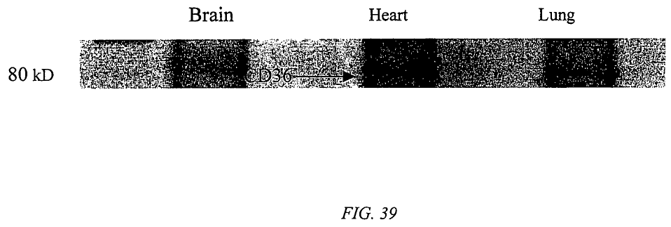

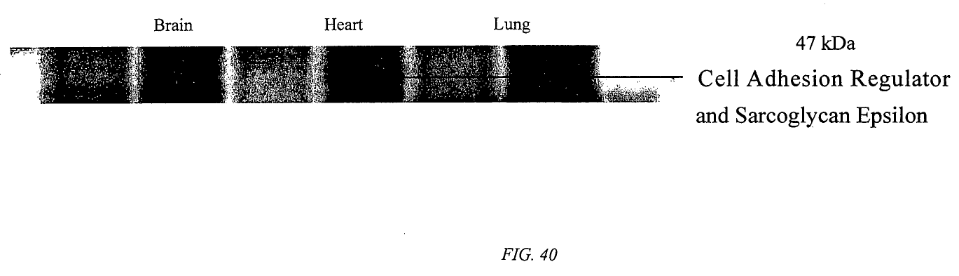

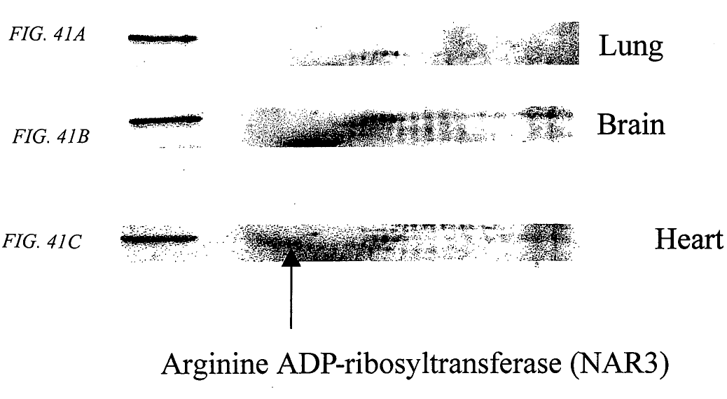

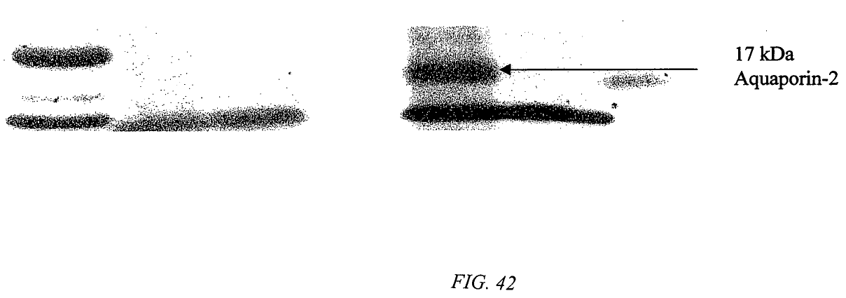

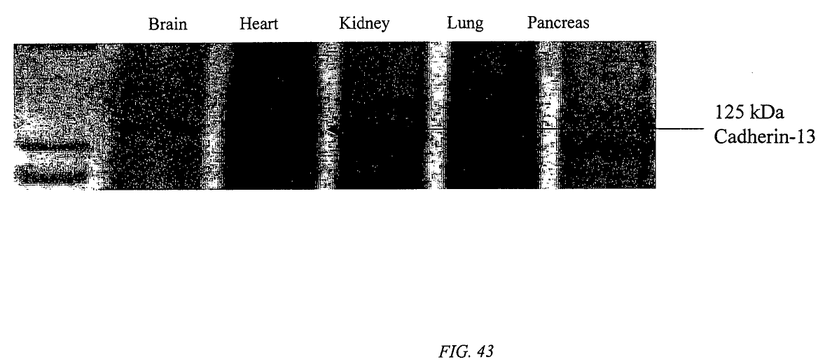

In subsequent studies, polypeptide profiles obtained from pig heart (cardiac tissue) were compared to the profiles of other tissues, such as brain and lung. In these comparisons, six proteins were found to be specific for heart tissue. FIG. 39 shows an approximately 80 kDa protein that appeared to be associated with the heart tissue but not brain or lung. FIG. 40 shows two approximately 47 kDa bands that are also specific for heart tissues. FIGS. 41A-C show the presence of an approximately 55 kDa polypeptide that is not associated with either the lung or the brain. Additionally, an approximately 17 kDa and an approximately 125 kDa were found to be present in the heart but in none the other tissues examined (see FIGS. 42 and 43, respectively).

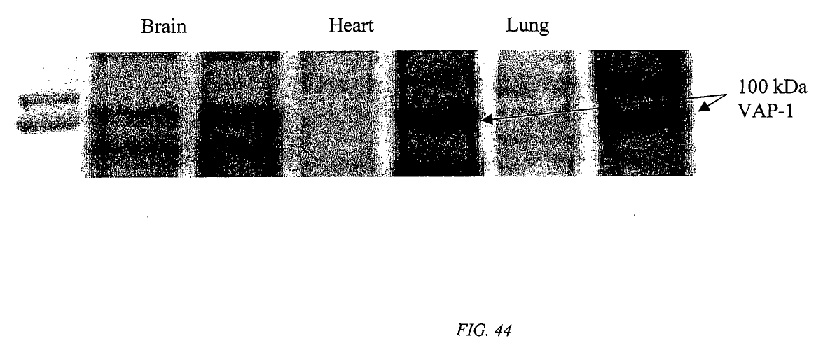

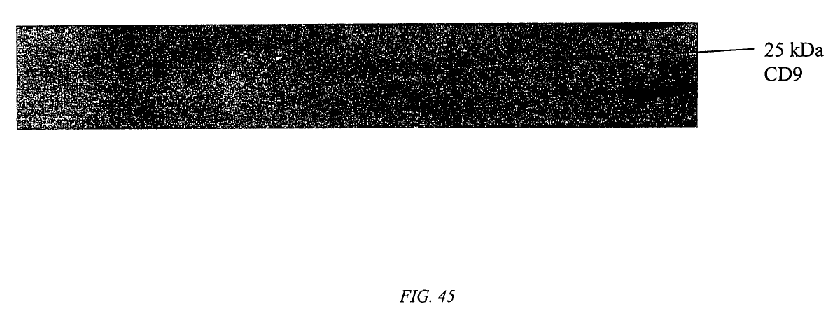

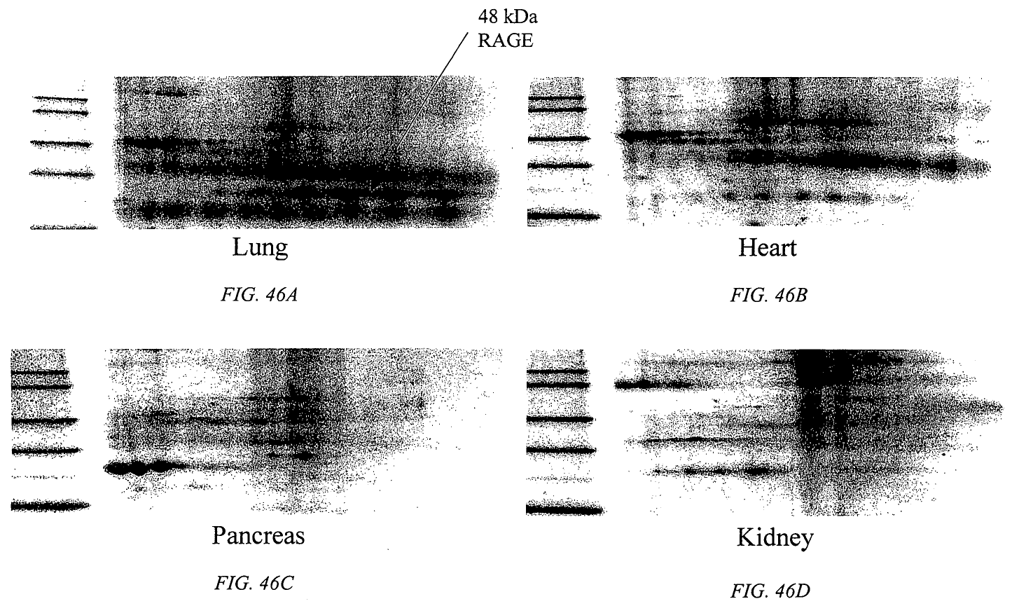

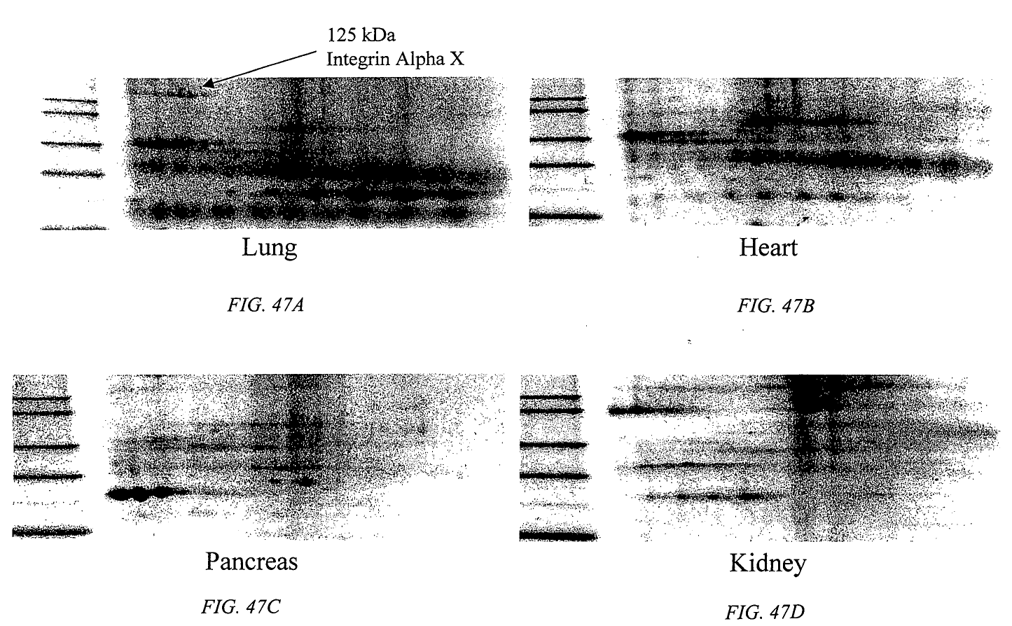

Lungs were also studied to identify any potential tissue-specific cell surface polypeptides associated with pulmonary tissues. FIG. 44 shows an approximately 100 kDa protein that is present in association with lung and heart tissue. FIG. 45 shows a polypeptide at about 25 kDa the was found to be present only in lung tissue. FIGS. 46A-D show the presence of a 48 kDa polypeptide that was similarly found only in lung tissue. A 125 kDa polypeptide that was present only in lung tissue is shown in FIGS. 47A-D.

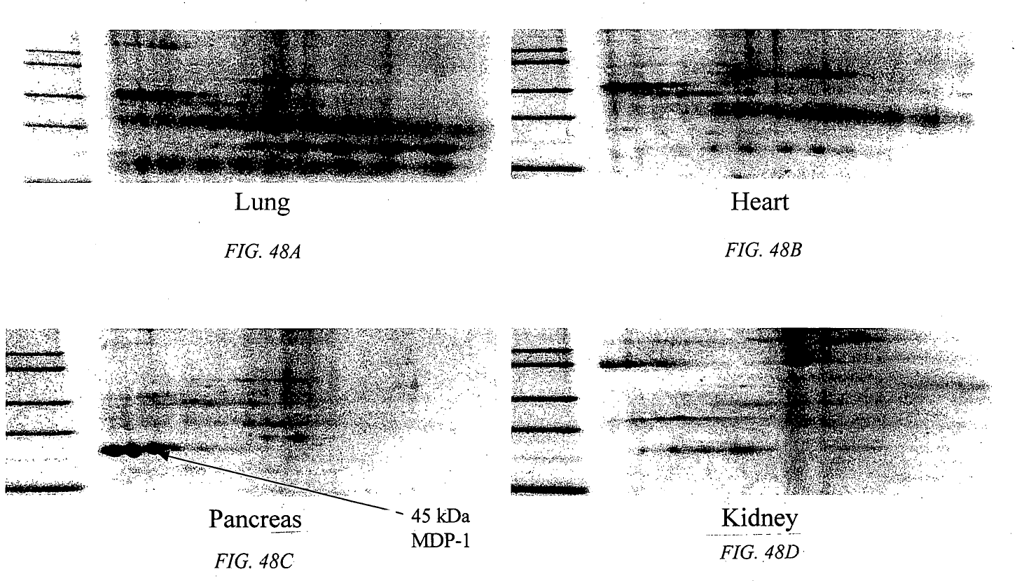

In other studies, pancreas tissue was examined to identify any luminally-exposed polypeptides associated therewith. An approximately 45 kDa luminally-exposed polypeptide having an isoelectric point between pH and 5 and 6 was found to be localized only to pig pancreatic tissue (see FIGS. 48A-D).

As demonstrated by these stained gels, the expression of isolated luminally exposed polypeptides in a variety of perfusible tissue types can be directly compared. More specifically, luminally-exposed proteins specific for a given tissue or a limited number of tissues can be readily isolated and identified by using the methods of Examples 23 and 24.

### Example 25

### Determination of the Amino Acid Sequence of Tissue-Specific Polypeptides

The following example describes the methods used to determine either N-terminal amino acid sequence or internal peptide fragment sequence for each of the tissue-specific proteins isolated as described in Examples 23 and 24.

After electrophoresis, proteins were transfered from the gel to a polyvinylidene difluoride (PVDF) membrane then stained with Coomassie Brilliant Blue. Polypeptide bands (or spots in the case of two-dimensional gel electrophoresis) that were present in only one or a few of the analyzed tissue types were excised from the membrane for protein sequence determination. For most of the excised polypeptides, N-terminal protein sequenece was obtained using Edman degradation. Proteins having a blocked N-terminus were digested by incubating the excised membrane containing the polypeptide of interest with approximately 150 ng of trypsin in the presence of 1% zwittergent 3-16 for approximately 20 hours. The tryptic fragments were separated using microbore HPLC. Selected fragments were then subjected to Edman degradation.

For each of the polypeptides that were isolated and sequenced, Table 1 displays the SEQ ID NO., molecular mass, organism from which the polypeptide was isolated, tissue specificity and type of peptide sequence that was obtained.

TABLE 1

| | | Tissue | Molecular Mass | |
|---|---|---|---|---|
| SEQ ID NO. | Organism | Specifity | (kDa) | Sequence Type |
| SEQ ID NO.: 70 | Pig | Brain | 40 | N-terminal |
| SEQ ID NO.: 71 | Pig | Brain | 85 | N-terminal |
| SEQ ID NO.: 72 | Pig | Brain | 35 | N-terminal |
| SEQ ID NOs.: 73 & 74 | Pig | Heart | 80 | Tryptic fragments |
| SEQ ID NO.: 75 | Pig | Heart | 47 | N-terminal |
| SEQ ID NO.: 76 | Pig | Heart | 47 | N-terminal |
| SEQ ID NO.: 77 | Pig | Heart | 55 | N-terminal |
| SEQ ID NO.: 78 | Pig | Heart | 17 | N-terminal |
| SEQ ID NOs.: 79 & 80 | Pig | Heart | 125 | Tryptic fragments |
| SEQ ID NO.: 81 | Pig | Lung | 25 | N-terminal |
| SEQ ID NO.: 82 | Pig | Lung | 48 | N-terminal |
| SEQ ID NO.: 83 | Pig | Lung | 125 | N-terminal |
| SEQ ID NO.: 84 | Pig | Lung | 25 | N-terminal |
| SEQ ID NOs.: 85 & 86 | Pig | Lung/Heart | 100 | Tryptic fragments |
| SEQ ID NO.: 87 | Pig | Pancreas | 45 | N-terminal |

### Example 26

### Comparison of the Sequences of Isolated-Tissue-Specific Polypeptides to Known Protein Sequences

The following example describes the methods used to determine the functional identity of the tissue-specific luminally-exposed polypeptides that were sequenced using the methods described in Example 25.

The amino acid sequence obtained for each tissue-specific luminally-exposed polypeptide was compared to amino acid sequences available in public databases. The amino acid sequence of both N-terminal and tryptic peptide fragments identified in the above examples were analyzed using MS PATTERN ver. 4.0.0, which is available at prospector.ucsf.edu. Specifically, each fragment was used as a query sequence against various publicly available protein sequence databases, such as the NCBI non redundant (nr) database, SwissProt and Owl. For each fragment, the database set was restricted to proteins having a molecular mass within about +/−25 kDa of the molecular mass of the protein from which the query fragment was obtained. Further specificity was obtained by requiring the N-terminal query sequences align near the N-terminus of a matching database sequence. If the N-terminal query sequence matched within 60 amino acids of the N-terminus of a database sequence, the N-terminal portion of the database sequence was further analyzed by using the program SIGNALP to determine the location of any N-terminal signal sequences and cleavage sites.

For each of the sequenced fragments, the first query of the analysis required that the amino acid sequence of the fragment exactly match a database sequence. If no match was obtained from the first query, successive iterations were performed until a sequence match was obtained for most of the fragments analyzed. A match was considered significant only if the aligned portions of the polypeptides displayed at least 60% o sequence identity. When tryptic sequence fragments were used as query sequences, both sequence fragments were required to match the same database protein at level of at least 60% identity. Those sequence fragments that had less than 60% sequence identity to a polypeptide in the database were considered to be unmatched.

Table 2 displays the results of the database comparisons using each amino acid sequence (SEQ ID NO.) listed in Table 1 as a query sequence.

TABLE 2

| | Homologous Protein | | |
|---|---|---|---|
| SEQ ID NO. | Sequence | NCBI Accession No. | Percent Identity |
| SEQ ID NO.: 70 | Folate Binding Protein | 4928859 | 100 |
| | (Human) | | |
| SEQ ID NO.: 71 | Unmatched | N/A | N/A |
| SEQ ID NO.: 72 | Unmatched | N/A | N/A |
| SEQ ID NO.: 73 | CD36 (Human) | 159613 | 80 |
| SEQ ID NO.: 74 | CD36 (Human) | 159613 | 100 |
| SEQ ID NO.: 75 | Cell Adhesion Regulator | AAD00260 | 89 |
| | (Rat) | | |
| SEQ ID NO.: 76 | Sarcoglycan Epsilon | 043556 | 100 |
| | (Human) | | |
| SEQ ID NO.: 77 | NAR3 (Human) | Q13508 | 80 |
| SEQ ID NO.: 78 | Aquaporin 2 (Dog) | CAA71663 | 83 |
| SEQ ID NO.: 79 | Cadherin-13 (Human) | NP001248 | 100 |
| SEQ ID NO.: 80 | Cadherin-13 (Human) | NP001248 | 100 |
| SEQ ID NO.: 81 | CD9 (Human) | XP_033314 | 100 |
| SEQ ID NO.: 82 | RAGE (Cow) | Q28173 | 80 |
| SEQ ID NO.: 83 | Intergrin Alpha-X (Human) | P20702 | 86 |
| SEQ ID NO.: 84 | CD81 (Human) | XP_006475 | 100 |
| SEQ ID NO.: 85 | VAP-1 (Human) | Q16853 | 100 |
| SEQ ID NO.: 86 | VAP-1 (Human) | Q16853 | 100 |
| SEQ ID NO.: 87 | MDP-1 (Human) | P16444 | 100 |

Table 3 displays the SEQ ID NOs. for each of the proteins identified from its source organism or a related organism. The SEQ ID NOs. for each of the corresponding polypeptide homologs identified from humans is also provided. Additionally, the SEQ ID NOs. of the polynucleotide sequences which encode each protein from the source or related organism and the corresponding human homolog are indicated. The term “N/A” in Table 3 means that the sequence was not available.

TABLE 3

| | | | DNA Encoding | |
|---|---|---|---|---|
| | Protein from | | Protein from | |
| | Source or | | Source or | DNA Encoding the |
| | Related | Homologous Human | Related | Homologous Human |
| Identified Protein | Organism | Protein | Organism | Protein |
| Folate Binding | SEQ ID | SEQ ID NO.: 89 | SEQ ID | SEQ ID NO.: 106 |
| Protein | NO.: 88 (Pig) | | NO.: 105 (Pig) | |
| CD36 | N/A | SEQ ID NO.: 90 | N/A | SEQ ID NO.: 107 |
| Cell Adhesion | SEQ ID | N/A | SEQ ID | N/A |
| Regulator | NO.: 91 (Rat) | | NO.: 108 (Rat) | |
| Sarcoglycan | N/A | SEQ ID NO.: 92 | N/A | SEQ ID NO.: 109 |
| Epsilon | | | | |
| NAR3 | N/A | SEQ ID NO.: 93 | N/A | SEQ ID NO.: 110 |
| Aquaporin 2 | SEQ ID | SEQ ID NO.: 94 | SEQ ID | SEQ ID NO.: 112 |
| | NO.: 94 (Dog) | | NO.: 111 | |
| | | | (Dog) | |
| Cadherin-13 | N/A | SEQ ID NO.: 96 | N/A | SEQ ID NO.: 113 |
| CD9 | N/A | SEQ ID NO.: 97 | N/A | SEQ ID NO.: 114 |
| RAGE | SEQ ID | SEQ ID NO.: 99 | SEQ ID | SEQ ID NO.: 116 |
| | NO.: 98 (Cow) | | NO.: 115 | |
| | | | (Cow) | |
| Integrin Alpha-X | N/A | SEQ ID NO.: 100 | N/A | SEQ ID NO.: 117 |
| CD81 | N/A | SEQ ID NO.: 101 | N/A | SEQ ID NO.: 118 |
| VAP-1 | N/A | SEQ ID NO.: 102 | N/A | SEQ ID NO.: 119 |
| MDP-1 | SEQ ID | SEQ ID NO.: 104 | SEQ ID | SEQ ID NO.: 121 |
| | NO.: 103 (Pig) | | NO.: 120 (Pig) | |

### Example 27

### Identification and Isolation of Polynucleotides that Encode Tissue-Specific Luminally-Exposed Polypeptides

The following provides exemplary methods that are used to identify and isolate polynucleotides that encode tissue-specific luminally-exposed polypeptides identified by the methods described herein.

Separate single stranded cDNA libraries (sscDNA) are constructed for each organism of interest. To create tissue-specific sscDNA libraries, portions of a tissue of interest from organisms, such as monkey, pig or rat, are excised and total RNA is isolated using methods commonly known in the art. For example, the commonly known guanidine salts/phenol extraction protocol is one of many methods which can be used to produce total RNA from isolated tissues. Chomczynski & Sacchi, 1987, Anal. Biochem. 162: 156. The total RNA extracts are then used to generate sscDNA using methods well known in the art. For example, an oligo dT primer flanked by two or more degenerate nucleotides at its 3′ end and a specific 15 to 21 base oligonucleotide at its 5′ end (RPBT), which is included to facilitate the binding of a reverse primer, can be used to prime first sscDNA synthesis from the preparations of total RNA.

The tissue-specific sscDNA is used as a template for PCR to obtain double-stranded cDNAs (cDNA) which contain the coding regions of the polypeptides identified using the methods described herein. Different cDNA cloning strategies are used depending on whether the tissue-specific luzninally-exposed polypeptide sequence that was obtained using the methods described herein matches a polypeptide sequence contained in publicly available databases. In cases in which a database match is found, the full-length DNA which encoded the polypeptide is often available. Such full-length DNA sequences can be used to design specific PCR primers which correspond to the 5′ and 3′ ends of the polypeptide coding sequence. These primers are then used to amplify the corresponding full-length cDNA using a high fidelity polymerase (e.g. pfu) and the sscDNA library as template. To facilitate subsequent directional cloning of the full-length DNA into an expression vector, each primer contains an additional short nucleotide sequence at its 5′ end. The additional sequences are complementary to the overhanging sequence generated by a different restriction endonuclease.

All oligonucleotides used in these methods can be synthesized with an Applied Biosystems 394 DNA synthesizer using established phosphoramidite chemistry. Ethanol precipitated primers can be used for PCR without further purification.

Alternative cloning approaches can be used in those instances in which the sequence of the tissue-specific luminally-exposed polypeptides obtained using the methods described herein do not match a polypeptide sequence contained in publicly available databases. In cases where the N-terminus portion of the polypeptide of interest has been identified, a corresponding degenerate primer can be designed which includes all possible nucleotide sequence variations capable of encoding the identified N-terminal peptide sequence. This degenerate primer and a primer which corresponds to the RPBS (incorporated into the sscDNA during synthesis) are then used to amplify the full-length cDNA using a high fidelity polymerase (e.g. pfu) and the sscDNA library as template. As previously described herein, each of these primers may include additional sequences which facilitate subsequent directional cloning of the full-length cDNA.

In cases where N-terminal amino acid sequence is not available but one or more internal peptide sequences are present, RACE PCR is used to obtain the 5′ and 3′ ends of the full-length cDNA which encodes the polypeptide of interest. Methods for performing RACE PCR are well known in the art. (See Bertling, W. M., et al. (1993) PCR Methods Appl. 3: 95-99; Frohman, M. A. (1991) Methods Enzymol. 218: 340-362; PCR Protocols: A Guide to Methods and Applications, (M. A. Innis, ed.), Academic Press, San Diego, Calif. (1990)). Briefly, RACE PCR is based on the construction of a specialized cDNA library that includes primer binding templates located at each end of the double stranded cDNA. The primer binding template that is ultimately located at the 3′ end of the coding strand of the dscDNA (RPBT) is formed as described previously described herein. The primer binding template that is ultimately located at the 5′ end of the coding strand of the dscDNA (FPBT) is formed by blunt end ligation of an adapter to the dscDNA after the completion of second strand synthesis. If a small internal portion of the sequence of a specific cDNA that lies between the FPBT and the RPBT is known, the region of DNA between the FPBT and this internal sequence can be amplified. Likewise, the region between the RPBT and the internal sequence can also be amplified.

To obtain the full-length cDNA of interest by RACE, an internal peptide sequence fragment of a polypeptide of interest is used to design a degenerate oligonucleotide that includes all possible nucleotide sequence variations capable of encoding the identified internal peptide fragment. This degenerate primer and a primer which corresponds to the RPBT are then used to amplify a region of the cDNA between the internal primer and the 3′ terminus of the cDNA coding strand (3′ end fragment) using a high fidelity polymerase (e.g. pfu) and the RACE cDNA library as template. Subsequent to the amplification, Taq polymerase can be used to add a single adenine nucleotide to the 3′ ends of each strand of the double stranded PCR product to facilitate cloning. The 3′ end fragment is subjected preparative gel electrophoresis then purified from the gel using a commercially available kit (QiagenGel Extraction Kit, Qiagen Corp.) according to the manufacturer's instructions. The gel-purified, 3′ end fragment is then inserted into a T-tailed PCR cloning vector and ligated at 15° C. overnight using T4 DNA ligase (New England BioLabs, Beverly, Mass.). A portion of the ligation mixture is then used to transform competent Escherichia coli and 100 μl of the transformation mixture is plated onto Luria broth plates containing 100 μg/ml of ampicillin. Isolated ampicillin-resistant transformants are picked, and streaked to obtain single colony isolates. Plasmid DNA is then obtained from these single colony isolates. The presence of the insert in each construct can be confirmed by amplification of the cloned region using oligonucleotide primers flanking the insert site. Clones having the appropriate size inserts are then sequenced using a cycle sequencing dye-terminator kit with AmpliTaq DNA Polymerase, FS (ABI, Foster City, Calif.). The sequencing reaction products are run on an ABI Prism 377 DNA Sequencer.

Using the nucleotide sequence of the 3′ end fragment, a gene specific primer complementary to the coding strand of the cDNA can be designed. This gene specific primer and a primer that corresponds to the FPBT are then used in conjunction with the RACE cDNA library and high fidelity polymerase (e.g. pfu) to amplify a fragment that corresponds to a region of the cDNA between the internal primer and the 5′ terminus of the cDNA coding strand (5′ end fragment). This 5′ end fragment is processed as previously described for the 3′ end fragment so as to obtain nucleotide sequence.

Having knowledge of the nucleotide sequence of both the 5′ and 3′ ends of the full-length cDNA, one can design oligonucleotide primers that correspond to each end of the cDNA sequence. These primers can then be used to amplify the full-length cDNA using the RACE cDNA library and a high fidelity polymerase such as pfu polymerase.

### Example 28

### Identification of cDNAs Encoding Homologs of Tissue-Specific Luminally-Exposed Polypeptides

The following example describes methods that are used to obtain cDNAs which encode the homologs of the tissue-specific luminally-exposed polypeptides described herein, including cDNA which encodes polypeptides homologous to luminally-exposed polypeptides comprising an amino acid sequence selected from the group consisting of SEQ ID NOs.: 70-104.

Polynucleotides encoding homologous polypeptides may be obtained by screening a cDNA library constructed from an appropriate tissue of an organism other than the organism from which the tissue-specific luminally-exposed polypeptide was originally identified.

To identify a polynucleotide which encodes a polypeptide homologous to a luminally-exposed polypeptide comprising an amino acid sequence selected from the group consisting of SEQ ID NOs.: 70-104, an oligonucleotide probe is constructed using the appropriate full-length cDNA sequence described in Example 27 herein. Methods of oligonucleotide probe construction are well known in the art.

A cDNA library from an organism other than the organism from which the tissue-specific luminally-exposed polypeptide was originally identified is prepared. This library is then screened for a polynucleotides which hybridize with the probes described above and which encode polypeptide homologous to a luminally-exposed polypeptide comprising an amino acid sequence selected from the group consisting of SEQ ID NOs.: 70-104. The cDNA library containing the polynucleotide which encodes the homologous polypeptide from such other organism can be plated using methods known in the art. (J. Sambrook, E. F. Fritsch, and T. Maniatus, Molecular Cloning, A Laboratory Manual, 2d edition, Cold Spring Harbor, N.Y., (1989)). The polynucleotides are then transferred to and immobilized on nitrocellulose or other carrier. In order to identify a polynucleotide that is homologous with luminally-exposed polypeptide comprising an amino acid sequence selected from the group consisting of SEQ ID NOs.: 70-104, the carrier containing the library is incubated with the radiolabeled probe sequence for 1 hour at 6×SSC at 45° C. The carrier is then washed three times for 30 minutes each in 0.2×SSC with 0.1% SDS at 42° C. Polynucleotides to which the oligonucleotide probe hybridizes under these conditions are detected using X-ray film.

The hybridizing polynucleotides can then be isolated, cloned and sequenced using methods commonly known in the art. Once the sequence of the hybridizing polynucleotide is determined, this sequence can be used to obtain the full-length polynucleotide homolog using the methods previously described in Example 27. The full-length homolog is then compared to the polynucleotide from which the probe was constructed to determine the percent nucleotide identity. Using commonly available computer programs, such as the Wisconsin Package developed and distributed by the Genetics Computer Group, the amino acid sequence of the homologous polypeptide can be determined. The homologous polypeptide is then compared to the polypeptide encoded by the polynucleotide from which the probe was constructed to determine the percent similarity of the two polypeptide sequences.

Database searching can also be used to identify a polypeptide homologous to a luminally-exposed polypeptide which comprises an amino acid sequence selected from the group consisting of SEQ ID NOs.: 70-104. The polynucleotide which encodes polypeptide which comprises an amino acid sequence selected from the group consisting of SEQ ID NOs.: 70-104 is obtained using the method described in Example 27. This sequence or fragment thereof is then used as a query sequence against the polynucleotide sequences in the NCBI nonredundant sequence database. The database search and sequence comparison is performed by using the NCBI BLASTN 2.0.9 computer algorithm with the BLOSUM62 matrix and the default parameters except that filtering is turned off.

A polynucleotide which encodes a polypeptide homologous to a luminally-exposed polypeptide which comprises an amino acid sequence selected from the group consisting of SEQ ID NOs.: 70-104 can be expressed, purified and used to generate antibodies thereto using the methods described herein.

### Example 29

### Expression and Purification of Recombinant Tissue Specific Luminally-Exposed Polypeptides and Fragments Thereof

The following example provides an exemplary method for the expression of tissue-specific luminally-exposed polypeptides (and fragments thereof) that are encoded by cDNA sequences identified by the methods described herein. This method is based on an E. coli expression system; however, one of ordinary skill in the art will recognize that a variety of host organisms and expression systems exist that can be used to express these tissue-specific luminally-exposed polypeptides.

Several vector systems for protein expression in E. coli are well known and available to someone knowledgeable in the art. A full-length cDNA, which encodes a polypeptide of interest and which contains restriction endonuclease sequences appropriate for directional insertion of the coding sequences into the vector, can be inserted into any of these vectors and placed under the control of the promoter such that the coding sequences can be expressed from the vector's promoter. Alternatively, the full-length cDNA can be selectively digested or used as a template for the amplification of select fragments which can be placed under the control of a promoter in an expression vector. Vectors such as the pGEX and pET3 series vectors can be for such expression. (see, Gene Expression Technology (D. V. Goeddel, ed.), Methods Enzymol. vol. 185, Academic Press, San Diego, Calif. (1990)).

The expression vector is then transformed into DH5^{α} or some other E. coli strain suitable for the over expression of proteins. Transformation can be facilitated using the calcium chloride method, electroporation protocols, or any other method for introducing nucleic acids into E. coli that is known in the art. Positive transformants are selected after growing the transformed cells on plates containing an antibiotic to which the vector confers resistance.

In one embodiment of the invention, the protein is expressed and maintained in the cytoplasm as the native sequence. In another embodiment, the expression vector can include a targeting sequence which allows for differential cellular targeting, such as to the periplasmic space or to the exterior medium. In yet other embodiments, a protein tag is included that facilitates purification of the protein from either fractionated cells or from the culture medium by affinity chromatography. A skilled artisan will recognize that embodiments represented by translational fusions require that the cDNA coding sequence be linked to the fusion partner in the appropriate reading frame so that translation of the desired fusion protein results.

Expressed proteins, whether in the culture medium or liberated from the periplasmic space or the cytoplasm, are then purified or enriched from the supernatant using conventional techniques such as ammonium sulfate precipitation, PEG precipitation, immunoprecipitation, standard chromatography, immunochromatography, size exclusion chromatography, ion exchange chromatography, hydrophobic interaction chromatography, affinity chromatography, HPLC two-dimensional electrophoresis and preparative electrophoresis. (see, Guide to Protein Purification (M. V. Deutcher, ed.), Methods Enzymol. vol. 182, Academic Press, San Diego, Calif. (1990)). Alternatively, if the polypeptide is secreted from the host cell into the surrounding medium in a state that is sufficiently enriched, the polypeptide or fragment thereof may be used for its intended purpose without further purification. The purity of the protein product obtained can be assessed using techniques such as SDS PAGE.

Antibodies capable of specifically recognizing the protein of interest can be generated using synthetic peptides using methods well known in the art. See, Antibodies: A Laboratory Manual, (Harlow and Lane, Eds.) Cold Spring Harbor Laboratory (1988). For example, synthetic peptides can be injected into mice to generate antibodies which recognize the full-length polypeptide. Antibodies prepared using these peptide fragments can be used to purify the full-length polypeptide by using standard immunochromatography techniques.

In an alternative protein purification scheme, a polynucleotide encoding the tissue-specific luminally-exposed polypeptide of interest or portion thereof can be incorporated as a translational fusion into expression vectors designed for use in affinity-based purification schemes. In such strategies the coding sequence of the polynucleotide of interest or portion thereof is inserted in-frame with the gene encoding the other portion of the fusion polypeptide (the affinity handle). In some embodiments, the affinity handle is polyhistidine.

In other embodiments the affinity handle is maltose binding protein (MBP). A chromatography matrix having nickel (if polyhistidine affinity handles are used) or an antibody to MBP (if MBP affinity handles are used) attached thereto is then used to purify polypeptide fusion. Protease cleavage sites can be engineered between the polyhistidine gene or the MBP gene and the polynucleotide of interest, or portion thereof. Thus, during of subsequent to the final purification step, the polypeptide of interest can be separated from the affinity handle by proteolysis.

Expression and Purification of a Tissue-Specific Luminally-Exposed Polypeptide in E. coli

In this example, a tissue-specific luminally-exposed polypeptide is expressed as a recombinant glutathione-S-transferase (GST) fusion polypeptide in E. coli and the fusion polypeptide is isolated and characterized. Specifically, the polypeptide of interest, such as a polypeptide comprising an amino acid sequence selected from the group consisting of SEQ ID NOs.: 70-104, is fused to GST and this fusion polypeptide is expressed in E. coli, e.g., strain PEB 199. Expression of the GST-tissue-specific luminally-exposed polypeptide fusion protein in PEB 199 is induced with IPTG. The crude bacterial lysates of the induced PEB 199 strain, which contains the recombinant fusion polypeptide, is then passed over a column of glutathione beads. Elution of the bound tissue-specific luminally-exposed polypeptide is accomplished by using thrombin to cleave the peptide linker which separates the glutathione-S-transferase affinity handle from the polypeptide of interest. The purity of this recombinant tissue-specific luminally-exposed polypeptide is determined by subjecting a sample of the eluate to PAGE and silver staining the resulting gel.

### Example 30

### Preparation of Polyclonal Antibodies to Tissue Specific Luminally-Exposed Polypeptides or Fragments Thereof

The following example illustrates the preparation of polyclonal antibodies directed to a full-length tissue-specific luminally-exposed polypeptide or a fragment thereof identified using the methods described herein.

Polyclonal antibodies directed to a tissue-specific luminally-exposed polypeptide identified using the methods described herein are prepared by inoculating a host animal with the polypeptide of interest. The polypeptide comprising the inoculum is substantially pure, preferably comprising less than about 1% contaminant. To increase the immune response of the host animal, the polypeptide of interest is combined with an adjuvant. Suitable adjuvants include alum, dextran, sulfate, large polymeric anions, oil & water emulsions, e.g. Freund's adjuvant, Freund's complete adjuvant, and the like. The polypeptide of interest may also be conjugated to synthetic carrier proteins or synthetic antigens.

A variety of hosts can be immunized to produce the polyclonal antibodies. Such hosts include rabbits, guinea pigs, rodents, e.g. mice, rats, sheep, goats, and the like. The polypeptide of interest is administered to the host, usually intradermally, with an initial dosage followed by one or more, usually at least two, additional booster dosages. Following immunization, the blood from the host is collected, followed by separation of the serum from the blood cells. The immunoglobulin present in the resultant antiserum may be further fractionated using known methods, such as ammonium salt fractionation, DEAE chromatography, and the like.

Preparation of Polyclonal Antibodies to a Fragment of a Tissue-Specific Luminally-Exposed Polypeptide

New Zealand white female rabbits are used for the production of polyclonal antibodies to one or more fragments of a tissue-specific luminally-exposed polypeptide identified using the methods described herein. Specifically, peptides comprising an amino acid sequence selected from the group consisting of SEQ ID NOs.: 70-104 are used. A synthetic peptide corresponding to a 28 amino acid residue fragment of a polypeptide identified using the methods described herein is linked to Kehole Limpet Hemocyanin (KLH) for use as an antigen. Subdermal injection is carried out using 1 mg of KLH-linked peptide that has been emulsified in Freund's complete adjuvant. After 3 weeks the animals are bled and tested for reactivity. The animals are injected again after 3 weeks using 1 mg of KLH-linked peptide in Freund's incomplete adjuvant. Two weeks later the serum is tested. The serum that is obtained is then tested to determine it reactivity to the full-length polypeptide antigen.

### Example 31

### Localization of Tissue Specific Luminally-Exposed Polypeptides Using Polyclonal Antibodies

The antibody localization methods described in the following example can be used to verify the tissue specificity of luminally-exposed target molecules, including the tissue-specific luminally-exposed polypeptides identified using the methods described herein. In some cases, where the polypeptide of interest has been previously isolated, commercial antibodies may be available. In other cases, where the polypeptide of interest has not been previously characterized antibodies may be prepared using the methods described in Examples 27-30.

Experiments which demonstrate the tissue-specificity of a polypeptide can be performed both in vitro and in vivo. For example, Western blot is an in vitro method that can be used to confirm the tissue specificity of polypeptides separated by PAGE as described previously in Example 24. In vivo localization can be achieved by injecting the appropriate labeled antibody into a host animal. After a sufficient incubation time, tissues can be removed and examined to determine the localization of the label.

In Vitro Tissue-Specific Localization of Rat Transferrin Receptor

The transferrin receptor (CD71) is a luminally-exposed transcytotic receptor present on the surface of endothelial cells that line the capillaries of the brain. Friden, P. M., et al. (1991). PNAS 88:4771-5. Using the methods previously described herein, CD71 was shown to be expressed in a brain-specific manner. Cell-surface polypeptides isolated from brain, heart, kidney and lung tissues were separated by gel electrophoresis as described in Example 24. The separated polypeptides were then transferred to nitrocellulose by blotting at 25 milliamp overnight. The filter blots were then blocked with 2% BSA in TBS, 0.1% Tween-20 buffer for about one hour at RT. The blocking solution was removed and the OX-26 monoclonal antibody (Accurate), which is specific for CD71 (see, e.g., U.S. Pat. No. 6,004,814), contained in 0.2% BSA buffer was incubated with the blot for about one hour at RT. The filters were washed three times for about ten minutes in TBS-TWEEN then incubated with the “secondary” horse radish peroxidase (HRP)-labeled antibody. After washing three times, the blots were developed with ECL-PIUS™ (Amersham/Pharmacia) and photographed over UV light.

In polypeptide preparations from isolated brain tissue, a band at about 90 kDa corresponding to the monomeric form of CD71 was present. No bands were detected in the polypeptide preparations obtained from isolated rat heart, kidney or lung tissues. Such results show that CD71 is expressed specifically in the brain tissues.

In Vivo Tissue-Specific Localization of Rat Transferrin Receptor

In vivo localization studies with OX-26 antibody demonstrated that CD71 is only expressed in brain capillaries thus confirming the ability of the methods described herein to identify tissue-specific luminally-exposed polypeptides. For these localization studies, OX-26 and a control antibody of the same isotype but a different specificity (specific for albumin) were labeled with biotin. About 0.5 ml of a 1 mg/ml solution of each antibody was injected into the tail vein of separate rats under light anesthesia. The antibody was allowed to circulate for about thirty minutes after which time the animal was sacrificed and its organs/tissues were removed individually. Sections of each were made of each tissue by placing a small cube in embedding medium (HistoPrep™, Fisher), in a small plastic cube. This preparation was then immersed for about twenty seconds in 2-methylpentane which had been prechilled in liquid nitrogen. The frozen cubes were kept on dry ice until they were sectioned. The tissues were sectioned at five mm slices on a cryostat, air dried overnight and fixed in acetone for two min. The slides were then stained with streptavidin-HRP.

FIGS. 49A-D show the immunohistochemistry of tissue sections from a rat which was injected with either OX-26 or a control antibody. FIG. 49A is brain from a rat injected with OX-26, FIG. 49B is brain from a rat injected with the anti-albumin control antibody, FIG. 49C is lung from a rat injected with OX-26, FIG. 49D is lung from a rat injected with the anti-albumin control antibody. These results demonstrate that the antibody localized to the capillaries of the brain, and to no other tissue. Such specificity is particularly advantageous in that it is often difficult to find therapeutics which can cross the blood-brain barrier.

In Vivo Localization of CD81

In another experiment, 50 μg of biotinylated antibody specific for rat CD81 (clone eat2 from Research Diagnostics, Inc.) was administered by to adult rats by tail vein injection. Thirty minutes after the administration of the antibody, the rats were sacrificed and organs were prepared for immunohistochemistry as described above.

Tissue sections of heart and liver and other organs were analyzed. The biotinylated antibody was only seen associated with the endothelium of the lung.

The polypeptide sequence of human CD81 is provided as SEQ ID NO.: 101. The corresponding nucleotide sequence is SEQ ID NOs.: 118.

In Vivo Localization of Folate Binding Protein

Using a biotinylated antibody directed to rat folate binding protein (clone LK26 from Signet Pathology Systems) in conjunction with the in vivo administration and immunohistochemistry techniques described above, folate binding protein (FBP) was shown to be tissue specific.

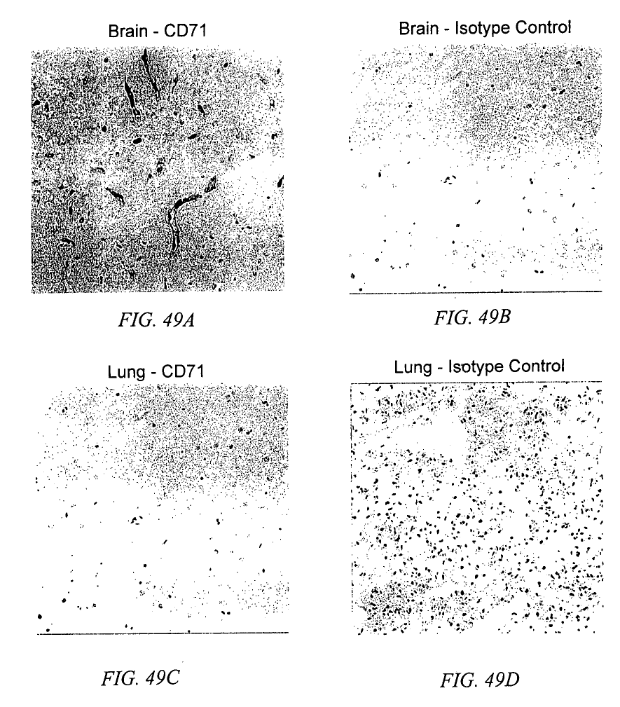

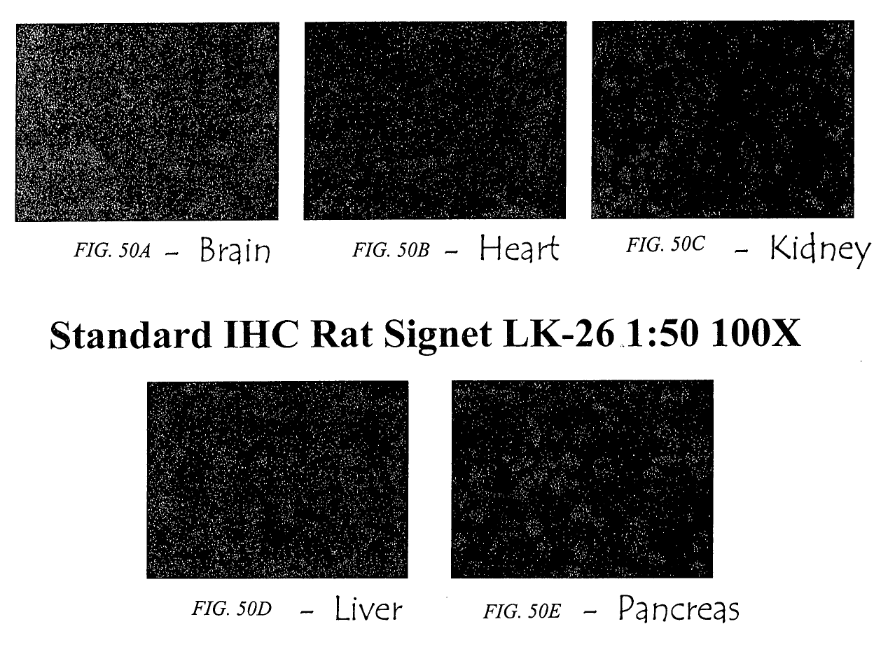

FIGS. 50A-E show the localization of the biotinylated antibody specific for FBP to the cells of the choroid plexus of the brain. Binding of the FBP specific antibody is not observed in any other tissues that were tested including heart, kidney, liver, and pancreas.

Although exemplary methods have be described for confirming the tissue specificity of polypeptides identified using the methods described herein, it will be appreciated that variations of the above-described methods can be utilized to confirm the tissue specificity of the polypeptides described herein.

### Example 32

### Tissue-Specific Delivery of a Therapeutic Moiety Linked to a Ligand

The following example describes the construction of a therapeutic moiety linked to a tissue-specific ligand and localization of the therapeutic moiety in a tissue-specific manner.

Localization of Toxin to the Brain Using OX-26 Antibody

In a follow-up experiment to the in vivo localization of CD71, OX-26 was used to deliver ricin A chain (Sigma, Catalog number L9514) to the choroid plexus of the brain. First, the ricin (therapeutic moiety) was mixed with the OX-26 antibody (ligand) and a disulfide-containing biotin (Pierce, catalog number 21331). The ricin and OX-26 were then linked by the addition of Nuetravidin (Pierce, catalog number 31000) which bound both biotins, thus forming a complex of ricin and the antibody. This therapeutic complex was then administered to rats through tail vein injection and brain and lung tissues were processed as described above.

It was found that the antibody not only facilitated the localization of the toxin to the vasculature of the brain, but presumably also its entry into the tissue via transcytosis. Once in the tissue, the toxin elicited an inflammatory response in the brain, a reaction, typically seen for any toxin introduced into the brain. No inflammatory response was seen in any other sectioned tissue.

Localization of Gentamicin to the Choroid Plexus Using Olate

Folate, which is a ligand for the transcytotic receptor folate binding protein, was selected as a ligand to illustrate the role of transcytosis in the delivery of therapeutic molecules to specific tissues. A therapeutic complex comprising folate linked to gentamicin (therapeutic moiety) was constructed. This therapeutic complex was then administered to rats through tail vein injection and colon, heart, kidney, liver, lung and brain tissues were processed as described above.

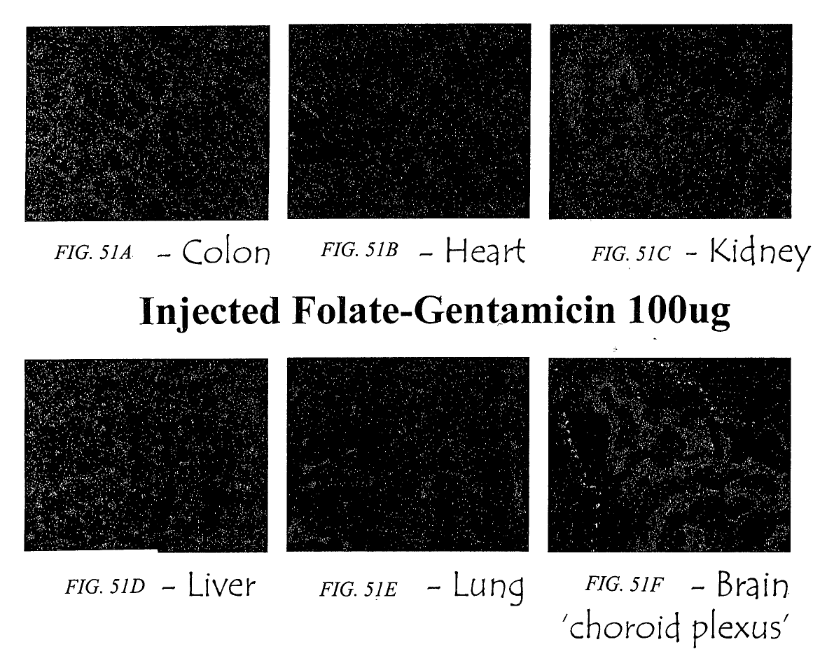

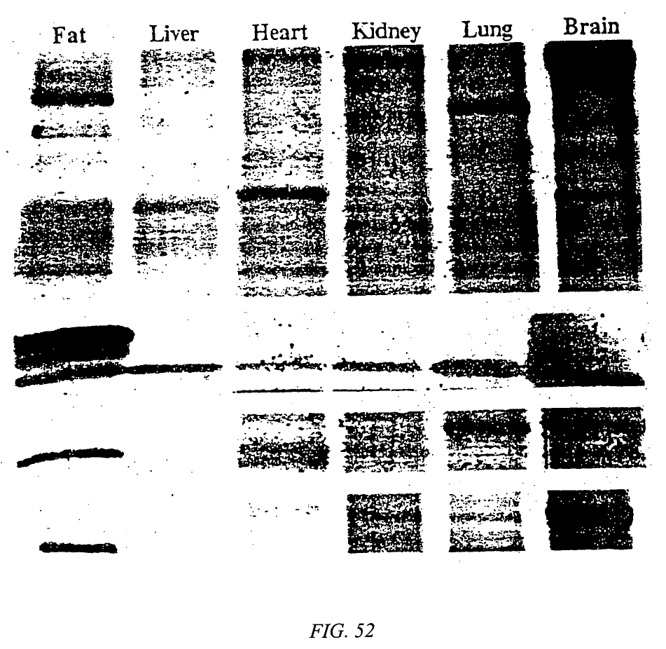

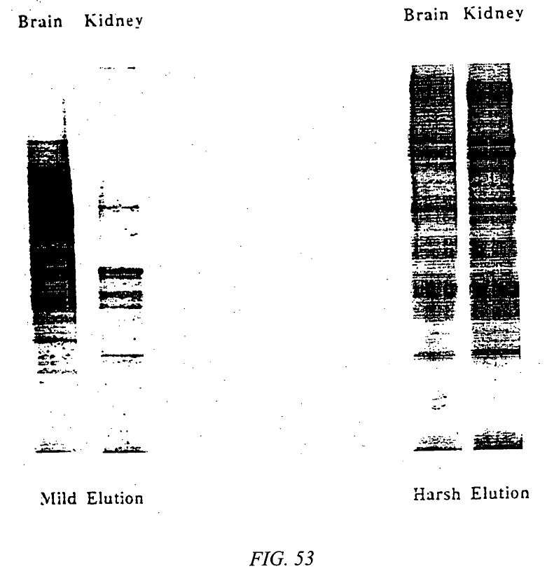

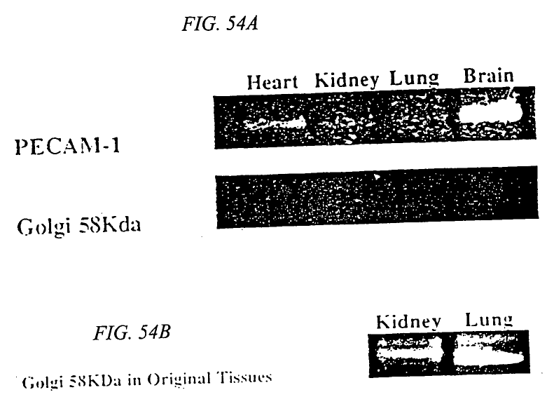

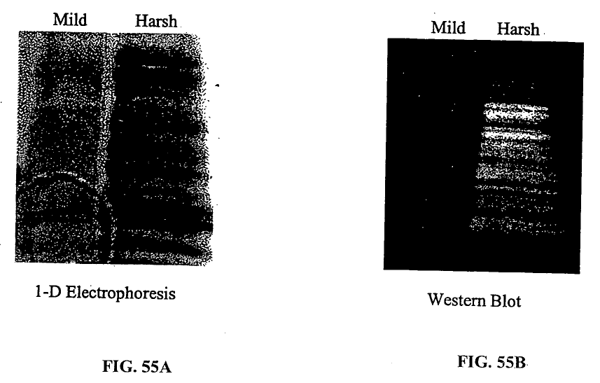

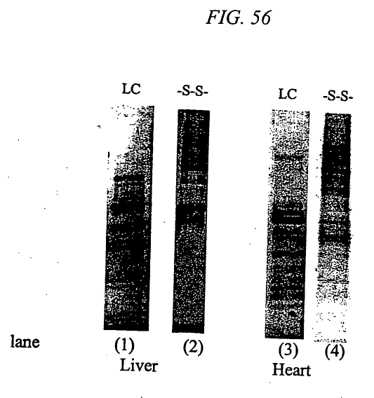

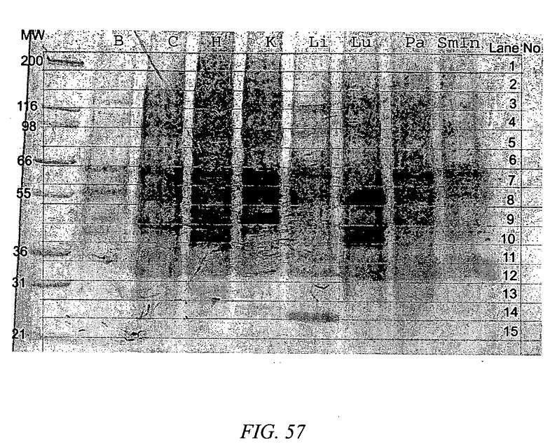

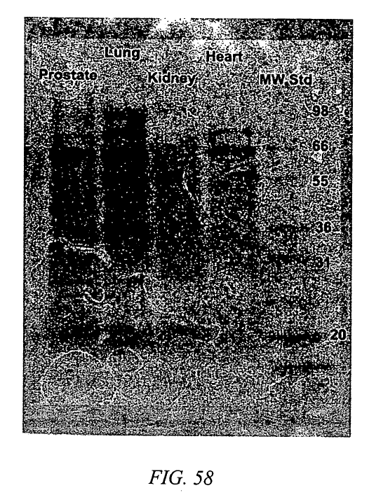

FIGS. 51A-F show that the therapeutic complex containing gentamicin localized only to the choroid plexus of the brain. No staining was observed for the other tissues examined. These results indicated that the ligand for folate binding protein FBP is useful as a tissue-specific ligand for therapeutic moieties and that the therapeutic moieties can be linked to folate without affecting its recognition of or specificity for its cell-surface target molecule. Furthermore, these results show that therapeutic moieties can be delivered across endothelial cell sheet that lines the vasculature thus permitting concentration of the therapeutic moiety in the underlying tissues.

Localization of Liposome Encgpsulated Molecules to the Brain Using an Antibody Specific for the Polypeptide Comprising SEQ ID NO.: 71 or a Homolog Thereto

The full-length cDNA which encodes the polypeptide comprising an amino acid sequence having SEQ ID NO.: 71 is used as a brain-specific target for the delivery of a liposome-encapsulated drug. The full-length cDNA which encodes the polypeptide comprising an amino acid sequence having SEQ ID NO.: 71 can be obtained using the methods described in Example 27. This cDNA is expressed, purified then used to generate polyclonal antibodies using the methods described herein. These polyclonal antibodies, which are specific for the cell-surface luminally-exposed polypeptide comprising an amino acid sequence having SEQ ID NO.: 71, are used as a ligand for the targeting of a therapeutic moiety to the brain in a tissue-specific manner.

The therapeutic moiety comprises gentamicin which is linked to the ligand via a liposomal linker. The liposomes are linked to the polyclonal antibody ligands through polyethylene glycol (PEG) molecules that are attached to phospholipids present at the surface of the liposome. To facilitate PEG-mediated antibody attachment, distearoylphosphatidylethanolamine (DSPE) is first derivatized with PEG having a molecule weight between 1000 and 5000 kDa then the free end of the attached PEG group is converted to a reactive maleimide using methods well known in the art, such as those described in U.S. Pat. No. 5,527,528. This reactive pegylated DSPE is incorporated into liposomes in about 0 to 10 mole percent. Other components of the liposome include unreactive pegylated DSPE in the range of about 0 to 10 mole percent, distearoylphosphatidylcholine (DSPC) or egg phosphatidylcholine in the range of 50 to 100 mole percent, and cholesterol in the range of about 0 to 50 mole percent.

Liposomes are formed by the reverse phase evaporation method described in U.S. Pat. No. 4,235,871. Gentamicin is entrapped in the liposomes by adding this compound in the aqueous phase during liposome formation.

It will be appreciated that liposomes can be produced by a variety of methods known in the art. For example, liposomes can be formed using the methods described in Storm et al., PSTT 1:19-31 (1998) and U.S. Pat. Nos. 4,522,803 and 4,885,172. It will also be appreciated that a variety of methods for encapsulating compounds within liposomes are known in the art. Such examples include the methods described in Mayer et al., Cancer Res. 49:59225930 and U.S. Pat. No. 4,885,172.

Gentamicin containing liposomes are linked to the polyclonal antibody specific to a polypeptide comprising an amino acid sequence having SEQ ID NO.: 71 by adding the antibody to the liposomes in a solution of phosphate buffered saline at pH 8.0 and incubating the suspension for 16 hours with gentle shaking under reducing conditions.

The liposome-linked antibodies are then intravenously administered to swine. After about 30 minutes, the animals are sacrificed and the brain, heart, and lung tissues are prepared as previously described. Gentamicin is expected to be found to accumulate only in the brain.

### Example 33

### Use of Anti-VAP-1/Doxorubicin Therapeutic Complex with an Acid Sensitive Linker for the Treatment of Lung Cancer

The following example describes the construction of an acid cleavable therapeutic complex that is formed between the anticancer agent doxorubicin and Fab2 fragments specific for VAP-1. Also described is a method of using this complex in the tissue-specific treatment of lung cancer.

Anti-VAP-1/doxorubicin therapeutic complexes can be constructed using the methods described in Example 32. Initially, a therapeutic level of a human anti-VAP1/doxorubicin complex is administered to a patient intravenously. An effective amount of the complex is delivered to the patient, preferably 1 pg to 100 mg/Kg of patient weight in saline or an intravenously acceptable delivery vehicle.

The anti-VAP-1 F(ab′)₂, which is used as the ligand, is specific for the lung tissue. As the therapeutic complex is taken up into the lung tissue, the acid sensitive linker is cleaved and the doxorubicin is free to intercalate into the DNA. Because the doxorubicin is incorporated into the DNA of cycling cells, the effect on the cancer cells which are in the process of cycling will be marked and the effect on the normal lung cancer cells much reduced. Therefore, the treatment results in a reduction of the number of cancer cells in the lung, with a minimum of side effects. Because doxorubicin generally targets dividing cells and, because of the tissue specificity, it will only affect the dividing cells of the lung, and therefore, it is envisioned that the number of cells killed due to side effects of the treatment will be minimal.

### Example 34

### Use of Anti-VAP-1/Doxocillin Therapeutic Complex for the Treatment of Lung Cancer Using a Prodrug

The following example describes a method of making an anti-VAP-1/doxocillin prodrug complex and a method of using this complex in the treatment for lung cancer.

The therapeutic complex is an anti-VAP-1/β-lactamase conjugate which includes an F(ab′)₂ specific for VAP-1 that is linked to β-lactamase via a polypeptide linker, or a covalent bond. An example of an appropriate polypeptide linker is SMCC. The therapeutic agent doxocillin does not cross the endothelium due to a number of negative charges in the structure, which makes it nontoxic for all cells and ineffective as an anticancer drug. However, doxocillin can be thought of as a pro-drug which becomes active upon cleavage of the β-lactam ring to produce doxorubicin. Doxorubicin does cross the endothelium and intercalates into the DNA of cycling cells, making it an effective chemotherapeutic agent.

Initially, a therapeutic amount of a anti-VAP-1/β-lactamase complex is administered to the patient intravenously. A therapeutic level of the therapeutic complex is administered to the patient at between about 1 μg to 100 mg/Kg of patient weight. The anti-VAP-1 F(ab′)₂ ligand, which is targeted to the lung tissue, is linked to the β-lactamase prodrug in the therapeutic complex using a linker which is not cleavale. After administration and localization of the therapeutic complex, a therapeutic level of doxocillin is administered to the patient at between about 1 μg to 100 mg/Kg of patient weight, preferably between 10 μg to 10 mg/Kg of patient weight. The doxocillin is taken up systemically, but only in the microenvironment of the lung, the doxocillin is cleaved by the β-lactamase to produce doxorubicin. Therefore, the eukaryotic cytotoxic activity of the prodrug is unmasked only at the location of the β-lactamase, that is, the lungs. The doxorubicin is taken up by the lung tissue and intercalates into the DNA. However, because the doxorubicin is incorporated into the DNA of cycling cells, the effect on the cancer cells which are in the process of cycling will be marked and the effect on the normal lung cancer cells much reduced. The treatment results in a reduction in the number of cancer cells in the lung.

### Example 35

### Use of Anti-VAP-1 Therapeutic Complex for the Treatment of Lung Infections

The following example describes the construction of a therapeutic complex comprising anti-VAP-1 linked to lipsomes containing cephalexin and a method of treating pneumonia using such a complex.

The most common bacterial pneumonia is pneumococcal pneumonia caused by Streptococcus pneumoniae. Other bacterial pneumonias may be caused by Haemophilus influenzae, and various strains of mycoplasma. Pneumococcal pneumonia is generally treated with penicillin. However, penicillin-resistant strains are becoming more common.

The present invention is used for the treatment of pneumococcal pneumonia in humans (or other mammals) as follows. A therapeutic complex is constructed by linking liposomes containing cephalexin to the F(ab′)₂ fragments of human antibodies directed to VAP-1. Polyethylene glycol (PEG) is used to join phosphotidylethanolamine (PE) in the outer lamella of the liposomes to the VAP-1 specific F(ab′)₂ fragments. The cephalexin is carried within the liposome. Such liposomes can be produced by using pegylated PE in the construction of the liposome using for example, the thin film hydration technique followed by a few freeze-thaw cycles. The cephalexin is captured within the interior of the liposome during liposome formation. The PEG on the exterior of the liposome is then activated as described above and anti-VAP-1 F(ab′)₂ fragments are linked thereto. Similar liposomal suspensions can also be prepared according to methods known to those skilled in the art.

A dispersion of the therapeutic complex is then prepared and 0.1 to 10 nmol is injected intravenously. The liposomes carrying the cephalexin are targeted to the lung by the VAP-1 specific F(ab′)₂ fragments. Upon binding to the endothelium, the liposomes are taken up and the cephalexin is taken into the lung tissue. The cephalexin can then act on the cell walls of the dividing S. pneumonia organisms. One advantage of the targeting of antibiotics to a specific region is that less antibiotic is needed for the same result, there is less likelihood of side effects, and the likelihood of contributing to the drug resistance of the microorganism is considerably reduced.

### Example 36

### Use of Anti-VAP-1 Therapeutic Complex for the Treatment of Tuberculosis

In the following example, a method is set out for the construction and use of a VAP-1/rifampin prodrug therapeutic complex to treat tuberculosis.

It can readily be envisioned that diseases such as tuberculosis, caused by the bacterium M. tuberculosis, which is often treated using rifampin or isoniazid for a very long period of time, would be more effectively treated using the therapeutic agent of the present invention. Much of the reason for the high incidence of disease and drug resistance in this microbe is the noncompliance with the extremely long course of treatment. It can be envisioned that using a method that directly targets the lungs with a high concentration of antibiotic would reduce the need for an unworkably long treatment and thus reduce the incidence of noncompliance and drug resistance.

The preferred embodiment is used for the treatment of tuberculosis in humans (or other mammals) as follows. A therapeutic complex is constructed by linking liposomes containing rifampin to the F(ab′)₂ fragments of human antibodies directed to VAP-1. PEG is used to join phosphotidylethanolamine (PE) in the outer lamella of the liposome to the VAP-1 specific F(ab′)₂ fragments. The rifampin is carried within the liposome. Such liposomes can be produced by using pegylated PE in the construction of the liposome using for example, the thin film hydration technique followed by a few freeze-thaw cycles. The cephalexin is captured within the interior of the liposome during liposome formation. The PEG on the exterior of the liposome is then activated as described above and anti-VAP-1 F(ab′)₂ fragments are linked thereto. Similar liposomal suspensions can also be prepared according to methods known to those skilled in the art.

A dispersion of the therapeutic complex is then prepared and 0.1 to 10 nmol is injected intravenously. The liposomes carrying the rifampin are targeted to the lung by the VAP-1 specific F(ab′)₂ fragments. Upon binding to the endothelium, the liposomes are taken up and the rifampin is taken into the lung tissue. The rifampin can then act on the M tuberculosis organisms.

### Example 37

### Use of Anti-VAP-1 Therapeutic Complex for the Treatment of Surfactant Deficiencies

The following example describes, a method for the synthesis and use of an anti-VAP-1/surfactant protein therapeutic complex to treat lung diseases resulting from underproduction of surfactant proteins.

A number of lung diseases, including emphysema, include, as part of the cause or effect of the disease, deficiencies of surfactant proteins. The present invention is used for the treatment of surfactant deficiencies as follows. A therapeutic complex is constructed by linking a surfactant protein, such as surfactant protein A (SP-A), to F(ab′)₂ fragments of antibodies directed to VAP-1. The bonding linking this therapeutic moiety with the ligand is a pH sensitive bond.

The therapeutic complex is then injected intravenously into a patient. The complex is targeted to the lung by the VAP-1 specific F(ab′)₂ fragments. After binding to the target, the therapeutic complex is taken up by the lung tissue and the change in pH cleaves the bond, thus releasing the surfactant protein.

### Example 38

### Use of Anti-VAP-1 Therapeutic Complex for the Treatment of Lung Transplantation Rejection

In the following example, a method is set out for the synthesis and use of a VAP-1/corticosteroid therapeutic complex to treat rejection of transplanted lung tissue.

The present invention is used for the treatment of lung transplantation rejection as follows. A therapeutic complex is constructed by linking an immunosuppressant, such as a corticosteroid or cyclosporin, to F(ab′)₂ fragments of VAP-1 specific antibodies using a pH sensitive linker.

This therapeutic complex is then injected intravenously into a patient and is targeted to the lung by the VAP-1 specific F(ab′)₂ fragments. After binding to the target, the therapeutic complex is taken up by the lung tissue and the change in pH cleaves the bond, thus releasing the immunosuppressant only in the area of the lungs. It can readily be seen that the advantage of such a treatment is that the patient is not immunosuppressed and still has a healthy active immune system during recovery from the surgery. The lung (or other transplanted organ) is the only organ which is immunosuppressed and is carefully monitored.

### Example 39

### Selective Isolation of Polypeptides Expressed in an Organ-Specific Manner on Vascular Endothelium

The following example demonstrates that the compositions and methods of the invention can be used to selectively isolate lumen-exposed molecules, such as polypeptides. In particular, this example demonstrates the selective isolation of a vascular endothelium lumen-exposed polypeptides from various organs of a rat, including brain, lungs, kidneys, hearts, liver, and omentum (fat).

In these experiments, male Fisher rats were used. Each rat was anesthetized by injection with 1.6 ml of ketamine:xylazine mixture (7.5 mg/ml ketamine: 5 mg/ml xylazine). A tracheotomy was then performed by inserting a catheter into the trachea of the rat and attaching this to a bulb to provide ventilation. The thorax of the animal was then opened and pericardium removed. 0.5 ml heparin (2000 units/ml) was injected into each of the left and right ventricles. A 14-gauge catheter was then attached to a perfusion line and inserted into the left ventricle. Although the amount of pressure was not critical, a range of between about 10 mm Hg and 80 mm Hg was used. Perfusion was at 20 mm Hg; an incision was made to the right atrium to permit flow of the perfusion buffer.

To clear the vasculature of blood, a buffer of 60 ml Ringers at pH 7.5 with nitroprusside at 0.1 mg/ml was perfused. Second, the vasculature was prepared for reaction with the cell membrane impermeant reagent by perfusion with 60 ml of borate-buffered saline at pH 9.0 (pH is not critical, a range of between about 7.5 and about 9.5 pH can be used with this particular reagent). Third, about 20 ml of this same buffer with the DTT cleavable reagent sulfosuccinimidyl-2-(biotinamido) ethyl-1,3-dithiopropioate (purchased as Sulfobiotin-X-NHS™ from Pierce Chemicals) was injected in the tissue and allowed to react for about one to two minutes (greater times and greater volumes can be successfully used). Concentrations of reagent from about 0.5 mg/ml to about 50 mg/ml can also be successfully used to label lumen-exposed-molecules.

After the reaction with reagent, 60 ml Ringers at pH 7.5 with 1.8 mg/ml glycine was perfused to remove excess biotin and to quench any remaining activated biotin. pH is not critical, a range of between about 7.5 and about 9.5 can be used. After this wash 60 ml of 0.25 M sucrose, 25 mM HEPES with 10 mg/ml of various protease inhibitors, including leupeptin, pepstatin, E64 and PMSF, to prevent proteolysis (the choice of protease inhibitors or their concentrations is not critical). Organs and tissues were then separately removed and stored at −80° C. until ready for use.

Homogenization was carried out by mincing a known weight of tissue with a razor blade. The minced tissue was placed in ten volumes (v/w) PBS at pH 7.4, 1.0 mM EDTA, 1.8 mg/ml glycine with a cocktail of protease inhibitors, including AEBSF, leupeptin, pepstatin A, bestatin, aprotinin (Sigma Cat. # P8340), E64 and PMSF. This was homogenized in a dounce homogenizer with about ten to twelve up and down strokes at approximately 1500 rpm. The homogenate was then centrifuged in about 20 ml aliquots at 500×g for ten minutes in order to remove cell debris and nuclei. The supernatant was removed and placed in a fresh tube. Each pellet was washed with about ten ml homogenization buffer and the spin repeated. Supernatants were pooled and spun at 40,000×g (or more) for about two hours to pellet the membrane fractions. Each of these pellets was resuspended in about ten ml homogenization buffer and re-homogenized as before. SDS and Triton X-100 detergents were then added to a final concentration of about 1% each to solubilize the cell membranes and release proteins.

These solubilized membrane protein fractions were aliquoted into 10 ml aliquots. Thirty of a 50% suspension of streptavidin beads (Pierce Chemicals) at 4 mg/ml binding capacity were added to each tube and this was inverted overnight at room temperature (RT). The beads were then allowed to settle into a pellet and the supernatant discarded. The pellet was washed five times with 1 ml homogenization buffer, 1% SDS, 1% Triton X-100 in order to remove non-specifically bound protein. Molecules modified with the biotin tag (i.e., the lumen-exposed vascular endothelial polypeptides bound to the membrane impermeable reagent) were specifically eluted from the beads by washing twice in (“mild conditions”) 50 ml homogenization buffer with 50 mM DTT, 1% SDS, 1% Triton-X 100; the DTT cleaved the internal disulfide domain of the membrane impermeable reagent, releasing the lumen-exposed vascular endothelial polypeptides and leaving the biotin bound to the immobilized streptavidin.

The eluted proteins were then precipitated with four volumes methanol, one volume chloroform and three volumes water, with mixing after each addition. The solution was centrifuged at 14,000 rpm for 5 minutes to separate the phases. The upper phase was removed and three volumes of methanol were added. The solution was centrifuged again to repellet the protein. The pellets were then resuspended in “sample buffer” comprising 83 mM Tris HCl, pH 6.8, 1% 2-mercaptoethanol (2-ME), 2% SDS, 10% glycerol, and boiled for 5 minutes (“harsh conditions”), after which the sample were ready for reducing polyacrylamide gel electrophoresis (PAGE).

Each preparation (pellet boiled in sample buffer) was separated by PAGE on a 4 to 20% gradient gel (Novex). The electrophoresed polypeptides were then transferred to nitrocellulose by blotting at 25 milliamp overnight. Filters were blocked with 2% BSA in TBS, 0.1% Tween-20 buffer for about one hour at RT. The primary antibody was then added in 0.2% BSA buffer for about one hour at RT. The filters were washed three times for about 10 minutes in TBS-TWEEN and then incubated with the “secondary” horseradish peroxidase (HRP)-labeled antibody. After washing three times, the blots were developed with ECL-PIUS™ (Amersham/Pharmacia) and photographed over UV light.

Histologic analysis was also performed on the tissue sections. Prior to freezing of the perfused and isolated organs and tissues, a small cube (approximately one cm cubed) was cut off for histologic analysis. While the tissue section can be prepared by any known technique, in this case the cube was placed in tissue embedding medium (HistoPrep™, Fisher), in a small plastic cube. This was then immersed for about twenty seconds in 2-methylpentane which had been pre-chilled in liquid nitrogen. The frozen cubes were kept on dry ice until they were sectioned. The tissues were sectioned at five mm slices on a cryostat, air dried overnight and fixed in acetone for 2 minutes. Fluorescent tags could be examined directed from these sections (using a fluorescent microscope).

For the in vivo localization studies, 0.5 ml biotin-labeled antibody at one mg/ml was injected into the tail vein of a rat under light anesthesia. The antibody was allowed to circulate for about 30 minutes after which time the animal was sacrificed and its organs removed individually. Sections of each were made as described above. The slides were stained with streptavidin-HRP using standard immunohistochemical techniques to detect the presence of antibody.

A rat was perfused with fluorescein-linker NHS (Pierce Chemical) at 1 mg/ml. A second rat was removed perfused with buffer only as negative control. Following perfusion, the organs were removed and tissue sections were made of each. Localization of the fluorescein to the vascular lumen without penetrating into the tissue was confirmed by fluorescence microscopy. Capillaries from kidneys from the two rats (test and control) were compared (capillaries were also viewed by phase contrast microscopy). When viewed by fluorescence microscopy, the capillary in the buffer-perfused animal is no longer visible, since there is no fluorescent label bound to endothelium. In contrast, in the animal perfused with the fluorescein-linker NHS, the capillary is readily seen because of binding of the reagent to the lumen-exposed endothelium (the NHS-moiety binds non-specifically to lumen-exposed polypeptides). Because the reagent is membrane impermeable, the fluorescein is viewed as lining the walls of the capillaries; no fluorescence is viewed in the tissue surrounding the vessel.

As described above, rats were perfused with the DTT-cleavable reagent sulfosuccinimidyl-2-(biotinamido)ethyl-1,3-dithiopropioate. The reagent had an in situ incubation time averaging about 1.5 minutes. Organs were removed, tissue homogenized, and lumen-exposed molecules were isolated as described above. The isolated lumen-exposed molecules from ten brains (14 grams total), three lungs (five grams total), four kidneys (six grams total), four hearts (six grams total), five grams of liver and five grams of omentum (fat) were analyzed/isolated on a 4 to 20% gradient PAGE (1.5 hours at 150 volts). The resulting gel was stained with Gelcode Blue™ stain (Pierce Chemical) to visualize the polypeptides from the different organs separated on the PAGE, as shown in FIG. 1. As demonstrated by the stained gel, lumen-exposed organ-specific vascular membrane polypeptides can be directly visualized on the PAGE. Vascular membrane proteins specific for a given tissue or a limited number of tissues are readily visualized, and isolated, by this technique.

To demonstrate the presence of potential contaminating naturally biotinylated proteins still bound to the immobilized binding domain ligand (in this case, immobilized streptavidin), the beads (after cleaving of the cleavable domain and elution of the cleaved half of the membrane impermeant reagent containing the lumen-exposed molecule) were eluted under “harsh conditions,” i.e., boiled in sample buffer (described above). This treatment will wash off all molecules remaining bound to the immobilized streptavidin. These samples were separated by PAGE and the gel stained (as above), the results of which are shown in FIG. 2. These results demonstrate that there are significant amount of proteins (i.e., naturally biotinylated proteins and non-specifically bound polypeptides not eluted under “mild conditions”) remaining on the beads after reduction of the membrane impermeable reagent's disulfide moiety (the “cleavage domain”) and subsequent “mild conditions” elution off of the non-immobilized fraction. These results also demonstrate that the PAGE polypeptide profile of the second “harsh” elution (including the naturally biotinylated proteins) is significantly different from the profile of the first “mild” elution fraction, i.e., the fraction comprising substantially only lumen-exposed vascular endothelial polypeptides.

These results further demonstrate that the profiles obtained under mild conditions reveal significant differences between tissues, while profiles of proteins remaining on the matrix subsequently eluted using harsh conditions are nearly identical between tissues. Thus, tissue-specific or organ-specific differences will only be revealed using mild conditions that specifically elute labeled proteins while leaving contaminants bound to the matrix (these contaminants are eluted using harsh conditions). These results also demonstrate that the methods of the invention can generate a preparation substantially free of “contamination” by naturally biotinylated polypeptides. Use of a membrane impermeable reagent lacking a cleavable domain would not allow discrimination between labeled (“tagged”) lumen-exposed vascular proteins and contaminating biotinylated proteins.

To establish the purity of the membrane preparations, Western blots were carried out for proteins known to be lumen-exposed endothelial plasma membrane associated polypeptides and for polypeptides known to be expressed on membranes elsewhere in tissues. PECAM-1 (also known as CD31, or endoCAM) was selected for analysis because it is a molecule known to be expressed on the plasma membrane of endothelial cells and exposed to the lumen of blood vessels (see, e.g., U.S. Pat. No. 5,955,4430; Wakelin (1996) J. Exp. Med. 184:229-239). It should, therefore, be labeled and isolated by the methods of the invention. In contrast, the Golgi-expressed 58K polypeptide should not be seen in any of these fractions (see, e.g., Bashour (1998) J. Biol. Chem. 273:19612-19617). Lumen-exposed polypeptides isolated using the methods of the invention (from the rat heart, kidney, lung and brain organ preparations, as described above) were separated by PAGE and stained, as described above. As is demonstrated by the Western blot represented in FIG. 3A, rat heart, kidney, lung and brain preparations contained significant amounts of PECAM-1, while the same fractions contained no Golgi-expressed 58K polypeptide. These results further demonstrate that the isolation process of the invention is specific for lumen-exposed (in this case, vascular endothelium exposed) molecules.

To demonstrate that the methods of the invention can specifically isolate a known vascular lumen-exposed polypeptide, a Western blot (containing separate, lumen-exposed protein preparations from several rat organs, as described above) was carried out using the OX-26 monoclonal antibody (Accurate), which is specific for CD71, the transferrin receptor (see, e.g., U.S. Pat. No. 6,004,814), a polypeptide known to be expressed on vascular endothelial cells in the brain. The results demonstrated that the CD71 polypeptide recognized by the OX-26 antibody is expressed only in the brain preparation and not in the heart, kidney or lung preparations. In vivo labeling studies with anti-CD71 antibody confirmed that CD71 is only expressed in brain capillaries. OX-26 and an isotype (negative) control antibody were labeled with biotin. Each antibody was injected into separate rats (0.5 ml at 1 mg/ml) and allowed to circulate for about 30 minutes. Immunohistochemical staining of tissue sections revealed that the anti-CD71 antibody had localized to (i.e., bound specifically to, above background) the brain capillaries and did not specifically bind to capillaries in other organs or tissues. The isotype control did not localize to any tissue (no binding above background). Thus, these results also demonstrate that the methods of the invention can specifically isolate a tissue-specific or organ-specific vascular lumen-exposed polypeptide.

### Example 40

### Methods of the Invention Exclude the Significant Amounts of Naturally Biotinylated Polypeptides

The following example demonstrates that the methods of the invention, by using reagents which are cleavable under mild conditions, are superior to techniques which use non-cleavable reagents. This example demonstrates the advantages of the methods of the invention, which use a cell membrane impermeable reagent comprising a domain situated between a first polypeptide-reactive domain and a second biotin-comprising domain, wherein this third domain links the first domain to the second domain by a cleavable chemical moiety that will not cleave under in vivo conditions, but can be induced to cleave under defined “mild conditions.” Thus, rather than using the harsh conditions needed to elute biotin from its ligand (avidin or streptavidin) to separate the “tagged” lumen-exposed polypeptide from the immobilized fraction, the “tagged” lumen-exposed molecules can be eluted by cleaving the reagent under “mild conditions.”

As demonstrated below, the harsh conditions needed to elute non-cleavable reagents resulted in significant amounts of “contaminating” compositions in the eluate in the form of naturally biotinylated proteins (including, significantly, those not exposed to the lumen in vivo). Thus, use of non-cleavable “tagging” reagents made it impossible to selectively identify and isolate tagged lumen-exposed compositions.

Methods which use non-cleavable cell membrane impermeable reagents, e.g., as described, e.g., by De La Fuente (1997) Amer. J. of Physiol. 272:L461-L470, must use harsh conditions to separate the biotinylated polypeptide from the immobilized avidin. De La Fuente “tagged” lumen-exposed polypeptides in lungs by perfusing the pulmonary artery with the cell membrane impermeant, non-cleavable biotinylated reagent sulfosuccinimidyl 6-biotin-amido hexanoate, which labels amine groups of polypeptides. De La Fuente incubated reagent-reacted tissue homogenates with streptavidin-agarose beads. However, because the affinity between biotin and avidin is relatively strong (e.g., about 10⁻¹⁵ M⁻¹), to elute the biotinylated polypeptides from the streptavidin beads, harsh conditions had to be used as elution conditions. This resulted in significant amounts of “contamination” (i.e., non-lumen-exposed compositions) in the eluate in the form of non-specifically binding compositions, e.g., polypeptides and other molecules. In contrast, the methods of the invention, by using cleavable cell membrane impermeant reagents, can be used to make preparations of lumen-exposed molecules with significantly less “contamination” by naturally biotinylated compositions.

Materials and Methods:

These experiments were performed using essentially the same materials, reagents and protocols as described above; male Fisher rats were also used.

Results:

Rat livers perfused with buffer only were removed and homogenized. Membranes were isolated. Streptavidin beads were added to the membrane preparation to purify naturally biotinylated proteins. Streptavidin beads were added to the membrane preparation to purify naturally biotinylated proteins. In one experiment the beads were eluted using “milder” elution conditions and the eluted fraction analyzed by one-dimensional electrophoresis (PAGE) and Western blot, as shown in the left panels of FIGS. 4A and 4B. As demonstrated by this analysis, elution under mild conditions isolated virtually no “contaminating” proteins. Similarly, when this same buffer is used to cleave the immobilized cell membrane impermeant reagent and elute the “tagged” polypeptide in the methods of the invention, substantially all of the eluted proteins with be those bound to the reagent via the first domain, with “contaminating” naturally biotinylated polypeptides remaining bound to the immobile fraction.

In contrast, under conditions required to elute biotin from avidin, significant amounts of “contaminating” naturally biotinylated polypeptides were eluted. In another experiment, the beads were treated (“eluted”) by boiling in “harsh conditions,” as described above. Analysis by PAGE and Western blot, as shown in the right panels of FIGS. 4 and 4B, demonstrated that under harsh conditions many contaminating proteins eluted from the beads. The presence of these “naturally biotinylated” proteins makes it impossible to selectively isolated lumen-exposed molecules under harsh elution conditions.

Next, experiments were performed comparing the ability of sulfosuccinimidyl 6-biotinamido hexanoate (a non-cleavable membrane impermeable reagent, designated “LC” in FIG. 5) and a cell membrane impermeable reagent used in methods within the scope of the invention (sulfosuccinimidyl-2-(biotinamido)ethyl-1,3-dithiopropioate, designated as “—S—S—” in FIG. 5 (purchased as Sulfobiotin-X-NHS™ from Pierce Chemicals), with a DTT cleavable domain) were directly compared (using essentially the same materials, reagents and protocols as described above (in Example 39). The two reagent were perfused into intact animals and membranes from liver and heart were prepared, as described above. Both membrane preparations were reacted with bead-immobilized avidin. After several washings, each batch of beads was (first) eluted under “mild conditions” comprising 50 mM DTT, 1% SDS, 1% Triton-X 100. The beads were next eluted under “harsh conditions” (83 mM Tris HCl, pH 6.8, 1% 2-mercaptoethanol (2-ME), 2% SDS, 10% glycerol, and boiled for five minutes). The eluted proteins were separated by PAGE and stained (as described above); a representation of these gels is presented as FIG. 5.

The profiles of eluted proteins isolated with the two reagents were found to be significantly different (equal protein loads were used in each PAGE lane to allow comparison of the two reagents). Elution of beads reacted with sulfosuccinimidyl-2-(biotinamido)ethyl-1,3-dithiopropioate tagged samples under mild conditions showed no staining over background (expected results because mild conditions cannot elute the high affinity bond between biotin and avidin or streptavidin and the fact that this reagent has no cleavable domain). The samples were next eluted under “harsh conditions” and the eluates analyzed; FIG. 5, lanes 1 and 3, for liver and heart, respectively.

Elution of beads reacted with the cleavable reagent Sulfobiotin-X-NHS™ under mild conditions is shown as lanes 2 and 4, for liver and heart, respectively. As can be seen, the profiles of eluted proteins isolated with the two reagents are significantly different. This can be explained by significant levels of background when using harsh elution conditions (lanes 1 and 3) due to the presence of endogenously biotinylated proteins and the fact that proteins that non-specifically interact with the matrix (i.e., immobilized avidin beads) are eluted under harsh conditions. These results further demonstrate the superiority of the methods of the invention to isolate lumen-exposed molecules.

### Example 41

### Detection and Identification of Lumen Exposed Proteins

The luminal proteins of the vasculature of an entire pig were labeled with biotin as disclosed herein. The labeled proteins were isolated from each one of the following organs: brain, colon, heart, kidney, liver, lunch, pancreas and small intestine. The isolated proteins from each organ were run on a one-dimensional SDS-PAGE gel (4-20%). After electrophoresis, proteins were transferred from the gel to a polyvinylidene difluoride membrane (PVDF). The PVDF was stained with Coomassie Brilliant Blue and fixed with a grid. The PVDF is illustrated in FIG. 6. Each individual band from the grid was excised from the PVDF and cut into ˜1 mm² pieces. The Coomassie stain was washed from the PVDF with 1 ml of 0.1% triethylamine in methanol. The PVDF was then washed 2 more times with 1 ml of methanol. The PVDF was incubated with 11 mls of 25 mM ammonium bicarbonate (pH 8), 1% zwittergent 3-16 and 15 ng/ml modified trypsin (Promega), at 37° C. overnight. After incubation, the PVDF sample was sonicated for 10 minutes, and liquid from the digest was removed and placed into a clean tube. An additional 11 mls of 25 mM ammonium bicarbonate (pH 8), 1% zwittergent 3-16 was added to the PVDF segments. The segments were then sonicated again for 10 minutes. Liquid was removed and combined with previous liquid removed for a total of 22 ml of extract.

Following the trypsin digest, tryptic peptides are isolated by reverse phase HPLC. Twenty mls of extract from a tryptic digest were injected into an Applied Biosystems 173A microbore HPLC. Tryptic peptides were separated on a C18 reverse phase column with a linear 2-60% acetonitrile gradient (0.1% Trifluoroacetic acid) applied over 80 minutes. Peptide fractions were then collected onto a PVDF using the Applied Biosystems Microblotter.

Peptide fractions (chromatograms) from all tissues in the same lane of equivalent molecular weight were compared. Tissues with a unique chromatogram were selected for Edman Sequencing using Applied Biosystems Procise 494 cLC Sequencer System. The sequences were used to identify proteins by web based database searching (e.g., Protein Prospector). Sequences were identified using human, mouse and pig databases. Table IV is a summary of each polypeptide isolated and sequenced. Table 4, column 1 identifies each polypeptide by its name according to public database NCBI or Swiss Prot Protein Databases (Swiss Prot). Table 4, column 2 identifies an amino acid sequence for the polypeptide in column 1. More than one sequence may be provided. In parenthesis is the species from which the sequence is derived. Table 4, column 3 identifies the tissue specificity or organ specificity for each polypeptide. Table 4, column 4 identifies the molecular weight of each tissue-specific or organ-specific polypeptide identified. Table 4, column 5, identifies the amino acid sequences for unique tryptic peptides sequenced. Table 4, column 6 identifies the nucleic acid sequence of the protein for each species identified in parenthesis.

TABLE 4

| | Amino Acid | | | Tryptic | |
|---|---|---|---|---|---|
| Polypeptide | Sequence | Tissue Specificity | MW (kDa) | Peptides | Nucleic Acid Sequence |
| CD98 (4F2Ag) | SEQ ID NO: 1 | Kidney | 58 | SEQ ID | SEQ ID NO: 2 (human) |
| | (human) | | | NOs: 17-19 | |
| CD108 | SEQ ID NO: 3 | Kidney | 75 | SEQ ID | SEQ ID NO: 4 (mouse); |
| (Semaphorin) | (mouse); SEQ ID | | | NOs: 20-21 | SEQ ID NO: 6 (human) |
| | NO: 5 (human) | | | | |
| CD 10 (Neutral | SEQ ID NO: 7 | Kidney | 85 | SEQ ID | SEQ ID NO: 8 (human) |
| Endopeptidase) | (human) | | | NOs: 22-23 | |
| CD13 | SEQ ID NO: 9 | Kidney | 109 | SEQ ID | SEQ ID NO: 10 (porcine); |
| (Aminopeptidase N) | (porcine); SEQ ID | | | NOs: 24-26 | SEQ ID NO: 12 (human) |
| | NO: 11 (human) | | | | |
| Similar to | SEQ ID NO: 13 | Lung | 50 | SEQ ID | SEQ ID NO: 14 (human) |
| Ectonucleotide | (human) | | | NO: 27 | |
| Pyrophosphatase/Ph | | | | | |
| osphdiesterase 5 | | | | | |
| CD 73 (Ecto 5′ | SEQ ID NO: 15 | Colon | 64 | SEQ ID | SEQ ID NO: 16 (human) |
| Nucleotidase) | (human) | | | NOs: 28-29 | |

In another study, male Fisher rats were used to identify luminal exposed tissue-specific or organ-specific proteins. The luminal proteins of rat vasculature were labeled with biotin as disclosed herein. Subsequently, labeled proteins that were isolated from homogenized prostate, lung, kidney, and heart were subjected to a 1-D SDS-PAGE polyacrylamide gel electrophoresis. The proteins were then electroblotted from the gel to a PVDF membrane and stained with Coomassie Brilliant Blue. Staining results are illustrated in FIG. 8. Protein patterns were compared between each tissue and protein bands unique to a tissue were subjected to N-terminal Edman protein sequencing. The N-terminal sequence identified using this procedure is SEQ ID NO: 30. SEQ ID NO: 30 was then compared with publicly available protein databases. SEQ ID NO: 30 was found to be homologous to Na/K ATPase beta-1 subunit, which is a 35 kDa, prostate specific protein. In particular, SEQ ID NO: 30 was homologous to SEQ ID NO: 31 of a rat database having a nucleic acid sequence of SEQ ID NO: 32. SEQ ID NO: 30 was also homologous to SEQ ID NO: 33 from a human database having a nucleic acid SEQ ID NO: 34.

Results from this study are summarized in Table 5 below:

TABLE 5

| | | | | | |
|---|---|---|---|---|---|
| Na/K ATPase beta- | SEQ ID NO: 31 | Prostate | 35 | SEQ ID NO: | SEQ ID NO: 32 (rat); SEQ |
| 1 subunit | (rat); SEQ ID NO: | | | 30 | ID NO: 34 (human) |
| | 33 (human) | | | | |

One skilled in the art will appreciate that these methods and compositions are and may be adapted to carry out the objects and obtain the ends and advantages mentioned, as well as those inherent therein. The methods, procedures, and compositions described herein are presently representative of preferred embodiments and are exemplary and are not intended as limitations on the scope of the invention. Changes therein and other uses will occur to those skilled in the art which are encompassed within the spirit of the invention and are defined by the scope of the disclosure.

Those skilled in the art recognize that the aspects and embodiments of the invention set forth herein may be practiced separate from each other or in conjunction with each other. Therefore, combinations of separate embodiments are within the scope of the invention as disclosed herein.

All patents and publications mentioned in the specification are all incorporated herein by reference.

The invention illustratively described herein suitably may be practiced in the absence of any element or elements, limitation or limitations which is not specifically disclosed herein. It is recognized that various modifications are possible within the scope of the invention disclosed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the disclosure.

Other embodiments of the invention can be envisioned within the scope of the following claims.

What is claimed is:

1. A kidney-specific therapeutic complex comprising a ligand capable of selectively binding to kidney tissue, a therapeutic moiety, and a linker which links said ligand to said therapeutic moiety.

2. The kidney-specific therapeutic complex of claim 1 wherein said ligand is capable of selectively binding to a lumen exposed molecule on said kidney tissue.

3. The kidney-specific therapeutic complex of claim 2 wherein said lumen exposed molecule comprises a polypeptide.

4. The kidney-specific therapeutic complex of claim 1 wherein said ligand is selected from the group consisting of a protein, an antibody, an oligonucleotide, a peptide nucleic acid, a small or large organic or inorganic molecule, and a polysaccharide.

5. The kidney-specific therapeutic complex of claim 4 wherein said antibody is selected from the group consisting of a polyclonal antibody, a monoclonal antibody, a humanized antibody, an antibody fragment Fab, an antibody fragment Fab′, an antibody fragment F(ab′)₂, and a single chain Fv.

6. The kidney-specific therapeutic complex of claim 2 wherein said lumen-exposed molecule is selected from the group consisting of CD98, CD108, CD10, CD13, and homologs thereof.

7. The kidney-specific therapeutic complex of claim 1 wherein said ligand is capable of selectively binding to CD98 or a homolog thereof.

8. The kidney-specific therapeutic complex of claim 1 wherein said ligand is capable of selectively binding to a polypeptide having an amino acid sequence of SEQ ID NO 1 or a homolog thereof.

9. The kidney-specific therapeutic complex of claim 1 wherein said ligand is capable of selectively binding to CD108 or a homolog thereof.

10. The kidney-specific therapeutic complex of claim 1 wherein said ligand is capable of selectively binding to a polypeptide having an amino acid sequence of SEQ ID NO 3 or a homolog thereof.

11. The kidney-specific therapeutic complex of claim 1 wherein said ligand is capable of selectively binding to a polypeptide having an amino acid sequence of SEQ ID NO 5 or a homolog thereof.

12. The kidney-specific therapeutic complex of claim 1 wherein said ligand is capable of selectively binding to CD10 or a homolog thereof.

13. The kidney-specific therapeutic complex of claim 1 wherein said ligand is capable of selectively binding to a polypeptide having an amino acid sequence of SEQ ID NO 7 or a homolog thereof.

14. The kidney-specific therapeutic complex of claim 1 wherein said ligand is capable of selectively binding to CD13 or a homolog thereof.

15. The kidney-specific therapeutic complex of claim 1 wherein said ligand is capable of selectively binding to a polypeptide having an amino acid sequence of SEQ ID NO 9 or a homolog thereof.

16. The kidney-specific therapeutic complex of claim 1 wherein said ligand is capable of selectively binding to a polypeptide having an amino acid sequence of SEQ ID NO 11 or a homolog thereof.

17. The kidney-specific therapeutic complex of claim 1 wherein said linker is selected from the group consisting of a bond, a peptide, a liposome, and a microcapsule.

18. The kidney-specific therapeutic complex of claim 1 wherein said linker is cleavable.

19. The kidney-specific therapeutic complex of claim 18 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

20. The kidney-specific therapeutic complex of claim 1 wherein said linker is non-cleavable.

21. The kidney-specific therapeutic complex of claim 20 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

22. The kidney-specific therapeutic complex of claim 1 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

23. The kidney-specific therapeutic complex of claim 22 wherein said protein is an enzyme.

24. The kidney-specific therapeutic complex of claim 23 wherein said enzyme cleaves a prodrug.

25. The kidney-specific therapeutic complex of claim 22 wherein said oligonucleotide is selected from the group consisting of an interfering RNA, an mRNA, a DNA, or an antisense nucleic acid.

26. The kidney-specific therapeutic complex of claim 1 wherein said therapeutic moiety is selected from the group consisting of methylprednisolone, chlorambucil, dipyridamole, acetylsalicylic acid, cyclophosphamide, prednisone, plasmapheresis, anti-platelet inhibitors, corticosteroids, prednisone, cyclosporine, azathioprine, and cyclophosphadmide.

27. A pharmaceutical composition comprising a kidney-specific therapeutic complex of claim 1 and a pharmaceutically acceptable carrier.

28. A method of treating a patient having a kidney condition comprising administering to said patient a therapeutically effective amount of a kidney-specific therapeutic complex wherein said therapeutic complex comprises a ligand capable of selectively binding to kidney tissue, a therapeutic moiety, and a linker that links said ligand to said therapeutic moiety.

29. The method of claim 28 wherein said ligand is capable of selectively binding to a lumen exposed molecule on said kidney tissue.

30. The method of claim 29 wherein said lumen exposed molecule is a polypeptide.

31. The method of claim 29 wherein said lumen exposed molecule is selected from the group consisting of CD98, CD 108, CD10, CD13, and homologs thereof.

32. The method of claim 29 wherein said lumen exposed molecule is a polypeptide having an amino acid sequence selected from the group consisting of SEQ ID NO 1, SEQ ID NO 3, SEQ ID NO 5, SEQ ID NO 7, SEQ ID NO 9, SEQ ID NO 11, and homologs thereof.

33. The method of claim 28 wherein said linker is non-cleavable.

34. The method of claim 33 wherein said non-cleavable linker is selected from the group consisting of sulfosuccinimidyl 6-[alpha-methyl-alpha-(2-pyridylthio) toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-(4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

35. The method of claim 28 wherein said linker is cleavable.

36. The method of claim 35 wherein said cleavable linker is selected from the group consisting of: a linker cleavable under reducing condition, a linker cleavable under acidic condition, a linker cleavable by an enzyme, a linker cleavable under basic condition, and a photocleavable linker.

37. The method of claim 28 wherein said kidney condition is selected from the group consisting of: acute renal failure, albuminuria, Alport syndrome, amyloidosis, proteinuria, analgesic-associated kidney disease, bacterial infections, Berger's disease, bile nephrosis, bladder and renal cell cancer, chronic renal failure, congenital nephrotic syndrome, cyst, cystine stones, cystitis, edema, enuresis, Ellis type II, focal and segmental hyalinosis, focal glomerulonephritis, Formad's kidney, fungal and parasitic infections, glomerulosclerosis, Goodpasture's syndrome, hypertension, hypervolemia, hypercalciuria, hyperoxaluria, IgA nephropathy, incontinence, interstitial nephritis, kidney transplant rejection, kidney cancer, lupus nephritis, membranoproliferative glomerulonephritis, membranous nephropathy, mesangial proliferative glomerulonephritis, nephrogenic diabetes insipidus, nephropathy, nephrogenic diabetes insipidus, nephrolithiasis, nephrolithiasis, nil disease, polycystic kidney disease, poststreptococcal glomerulonephritis, proteinuria, pyelonephritis, rapidly progressive glomerulonephritis, renal allograft rejection, renal artery stenosis, renal cell carcinoma, reflux nephropathy, renal cell carcinoma, renal cysts, renal osteodystrophy, renal tubular acidosis, renal vein thrombosis, struvite stone, systemic lupus erythematosus, thrombotic thrombocytopenic purpura, transitional cell cancer, uremia, urolithiasis, vasculitis, vesico-ureteric reflux, viral infections, Wegener's granulomatosis, and Wilm's tumor.

38. The method of claim 28 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

39. The method of claim 28 wherein said therapeutic moiety is selected from the group consisting of methylprednisolone, chlorambucil, dipyridamole, acetylsalicylic acid, cyclophosphamide, prednisone, plasmapheresis, anti-platelet inhibitors, corticosteroids, prednisone, cyclosporine, azathioprine, and cyclophosphadmide.

40. The method of claim 28 wherein said therapeutic complex is administered by means selected from the group consisting of orally, parenterally by inhalation, topically, rectally, ocularly nasally, buccally, vaginally, sublingually, transbuccally, liposomally, via an implanted reservoir, and via local delivery.

41. A method of determining the presence or concentration of CD98 or a homolog thereof in a tissue, organ, or cell comprising administering the therapeutic complex of claim 7 to said tissue, organ, or cell and identifying or quantifying the amount of bound therapeutic complex.

42. A method of determining the presence or concentration of CD108 or a homolog thereof in a tissue, organ, or cell comprising administering the therapeutic complex of claim 9 to said tissue, organ, or cell and identifying or quantifying the amount of bound therapeutic complex.

43. A method of determining the presence or concentration of CD10 or a homolog thereof in a tissue, organ, or cell comprising administering the therapeutic complex of claim 12 to said tissue, organ, or cell and identifying or quantifying the amount of bound therapeutic complex.

44. A method of determining the presence or concentration of CD13 or a homolog thereof in a tissue, organ, or cell comprising administering the therapeutic complex of claim 14 to said tissue, organ, or cell and identifying or quantifying the amount of bound therapeutic complex.

45. A lung-specific therapeutic complex comprising a ligand capable of selectively binding a lung specific molecule; a therapeutic moiety; and a linker that links said ligand to said therapeutic moiety.

46. The lung-specific therapeutic complex of claim 45 wherein said lung specific molecule is lumen exposed.

47. The lung-specific therapeutic complex of claim 46 wherein said lung specific molecule is a polypeptide.

48. The lung-specific therapeutic complex of claim 45 wherein said ligand is selected from the group consisting of a protein, an antibody, an oligonucleotide, a peptide nucleic acid, a small or large organic or inorganic molecule, and a polysaccharide.

49. The lung-specific therapeutic complex of claim 48 wherein said antibody is selected from the group consisting of a polyclonal antibody, a monoclonal antibody, a humanized antibody, an antibody fragment Fab, an antibody fragment Fab′, an antibody fragment F(ab′)₂, and a single chain Fv.

50. The lung-specific therapeutic complex of claim 45 wherein said lung specific molecule is similar to Ectonucleotide Pyrophosphatase/Phosphodisesterase 5 or a homolog thereof.

51. The lung-specific therapeutic complex of claim 45 wherein said lung specific molecule is a polypeptide having an amino acid sequence of SEQ ID NO 13 or a homolog thereof.

52. The lung-specific therapeutic complex of claim 45 wherein said linker is selected from the group consisting of a bond, a peptide, a liposome, and a microcapsule.

53. The lung-specific therapeutic complex of claim 45 wherein said linker is cleavable.

54. The lung-specific therapeutic complex of claim 53 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

55. The lung-specific therapeutic complex of claim 45 wherein said linker is non-cleavable.

56. The lung-specific therapeutic complex of claim 55 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

57. The lung-specific therapeutic complex of claim 45 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

58. The lung-specific therapeutic complex of claim 57 wherein said protein is an enzyme.

59. The lung-specific therapeutic complex of claim 58 wherein said enzyme cleaves a prodrug.

60. The lung-specific therapeutic complex of claim 45 wherein said therapeutic moiety is selected from the group consisting of α-adrenergic agents, theophylline, corticosteroids, cromolyn sodium, and anticholinergic agents.

61. A pharmaceutical composition comprising a lung specific therapeutic complex of claim 45 and a pharmaceutically acceptable carrier.

62. A method of treating a patient having a pulmonary condition comprising administering to said patient a therapeutically effective amount of a lung-specific therapeutic complex wherein said therapeutic complex comprises a ligand capable of selectively binding to lung tissue, a therapeutic moiety, and a linker that links said ligand to said therapeutic moiety.

63. The method of claim 62 wherein said ligand is capable of selectively binding to a lumen exposed molecule on said lung tissue.

64. The method of claim 63 wherein said lumen exposed molecule is a polypeptide.

65. The method of claim 62 wherein said ligand is capable of selectively binding to a polypeptide similar to Ectonucleotide Pyrophosphatase/Phosphodiesterase 5.

66. The method of claim 62 wherein said ligand is capable of selectively binding to a polypeptide having an amino acid sequence of SEQ ID NO 13 or a homolog thereof.

67. The method of claim 62 wherein said linker is selected from the group consisting of a bond, a peptide, a liposome, and a microcapsule.

68. The method of claim 62 wherein said linker is non-cleavable.

69. The method of claim 68 wherein said non-cleavable linker is selected from the group consisting of sulfosuccinimidyl 6-[alpha-methyl-alpha-(2-pyridylthio) toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

70. The method of claim 62 wherein said linker is cleavable.

71. The method of claim 70 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

72. The method of claim 62 wherein said pulmonary condition is selected from the group consisting of: asthma, acute respiratory disorder, acute bronchitis, atelectasis, bacterial infection, brinchiectasis, chronic obstructive pulmonary disease, cystic fibrosis, emphysema, fungal infection, parasitic infection, lung cancer, lung transplant rejection, pneumonia, pulmonary adenomatosis, pulmonary embolism, pulmonary hypertension, pulmonary thromboembolism, pulmonary edema, severe acute respiratory syndrome, and lung abscess.

73. The method of claim 62 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

74. The method of claim 62 wherein said therapeutic moiety selected from the group consisting of β-adrenergic agents, theophylline, corticosteroids, cromolyn sodium, and anticholinergic agents.

75. The method of claim 62 wherein said therapeutic complex is administered by means selected from the group consisting of orally, parenterally by inhalation, topically, rectally, ocularly nasally, buccally, vaginally, sublingually, transbuccally, liposomally, via an implanted reservoir, and via local delivery.

76. A method of determining the presence or concentration of a polypeptide similar to Ectonucleotide Pyrophosphatase/Phosphodiesterase 5 or a homolog thereof in a tissue, organ, or cell comprising administering the therapeutic complex of claim 50 to said tissue, organ, or cell and identifying or quantifying the amount of bound therapeutic complex.

77. A colon-specific therapeutic complex comprising a ligand capable of selectively binding a colon specific molecule, a therapeutic moiety, and a linker that links said ligand to said therapeutic moiety.

78. The colon-specific therapeutic complex of claim 77 wherein said colon specific molecule is lumen exposed.

79. The colon-specific therapeutic complex of claim 78 wherein said colon specific molecule is a polypeptide.

80. The colon-specific therapeutic complex of claim 77 wherein said ligand is selected from the group consisting of a protein, an antibody, an oligonucleotide, a peptide nucleic acid, a small or large organic or inorganic molecule, and a polysaccharide.

81. The colon-specific therapeutic complex of claim 80 wherein said antibody is selected from the group consisting of a polyclonal antibody, a monoclonal antibody, a humanized antibody, an antibody fragment Fab, an antibody fragment Fab′, an antibody fragment F(ab′)₂, and a single chain Fv.

82. The colon-specific therapeutic complex of claim 77 wherein said colon specific molecule is CD73 or a homolog thereof.

83. The colon-specific therapeutic complex of claim 77 wherein said colon specific molecule is a polypeptide having an amino acid sequence of SEQ ID NO 15 or a homolog thereof.

84. The colon-specific therapeutic complex of claim 77 wherein said linker is selected from the group consisting of a bond, a peptide, a liposome, and a microcapsule.

85. The colon-specific therapeutic complex of claim 77 wherein said linker is cleavable.

86. The colon-specific therapeutic complex of claim 85 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

87. The colon-specific therapeutic complex of claim 77 wherein said linker is non-cleavable.

88. The colon-specific therapeutic complex of claim 87 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

89. The colon-specific therapeutic complex of claim 77 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

90. The colon-specific therapeutic complex of claim 77 wherein said protein is an enzyme.

91. The colon-specific therapeutic complex of claim 90 wherein said enzyme cleaves a prodrug.

92. The colon-specific therapeutic complex of claim 77 wherein said therapeutic moiety is selected from the group consisting of corticosteroid therapy, anticholinergics, diphenoxylate, deodorized opium tincture, codeine, sulfasalazine, azodisalicylate, and 5-aminosalicylate, and 5-fluorouracil.

93. A pharmaceutical composition comprising a colon specific therapeutic complex of claim 77 and a pharmaceutically acceptable carrier.

94. A method of treating a patient having a colon condition comprising administering to said patient a therapeutically effective amount of a colon-specific therapeutic complex wherein said therapeutic complex comprises a ligand capable of selectively binding to lung tissue, a therapeutic moiety, and a linker that links said ligand to said therapeutic moiety.

95. The method of claim 94 wherein said ligand is capable of selectively binding to a lumen exposed molecule on said colon tissue.

96. The method of claim 95 wherein said lumen exposed molecule is a polypeptide.

97. The method of claim 94 wherein said ligand is capable of selectively binding to a CD73.

98. The method of claim 94 wherein said ligand is capable of selectively binding to a polypeptide having an amino acid sequence of SEQ ID NO 15 or a homolog thereof.

99. The method of claim 94 wherein said linker is selected from the group consisting of a bond, a peptide, a liposome, and a microcapsule.

100. The method of claim 94 wherein said linker is non-cleavable.

101. The method of claim 100 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-(4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

102. The method of claim 92 wherein said linker is cleavable.

103. The method of claim 100 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

104. The method of claim 94 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

105. The method of claim 94 wherein said therapeutic moiety is selected from the group consisting of corticosteroid therapy, anticholinergics, diphenoxylate, deodorized opium tincture, codeine, sulfasalazine, azodisalicylate, and 5-aminosalicylate, and 5-fluorouracil.

106. The method of claim 94 wherein said colon condition is selected from the group consisting of acute colitis, adenocarcinoma, cancer, carcinoid tumor of colon, collagenous colitis, colorectal cancer, Crohn's disease, cryptosporidiosis, colon cancer, diverticulosis of colon, dysentery, gastroenteritis, giardiasis, inflammatory bowel disease, intestinal parasite ascaris lumbricoides, irritable bowel syndrome, ischemic colitis, leiomyosarcoma of colon, peptic ulcer, pneumatosis intestinalis, polyposis coli, pseudomembranous colitis, squamous cell carcinoma of anus, toxic megacolon, tubulovillous adenoma, ulcerative colitis, tumors of the small intestine and villous adenoma.

107. The method of claim 94 wherein said therapeutic complex is administered by means selected from the group consisting of orally, parenterally by inhalation, topically, rectally, ocularly nasally, buccally, vaginally, sublingually, transbuccally, liposomally, via an implanted reservoir, and via local delivery.

108. A method of determining the presence or concentration of CD73 or a homolog thereof in a tissue, organ, or cell comprising administering the therapeutic complex of claim 82 to said tissue, organ, or cell and identifying or quantifying the amount of bound therapeutic complex.

109. A prostate-specific therapeutic complex comprising a ligand capable of selectively binding a prostate specific molecule, a therapeutic moiety, and a linker that links said ligand to said therapeutic moiety.

110. The prostate-specific therapeutic complex of claim 109 wherein said prostate specific molecule is lumen exposed.

111. The prostate-specific therapeutic complex of claim 110 wherein said prostate specific molecule is a polypeptide.

112. The prostate-specific therapeutic complex of claim 109 wherein said ligand is selected from the group consisting of a protein, an antibody, an oligonucleotide, a peptide nucleic acid, a small or large organic or inorganic molecule, and a polysaccharide.

113. The prostate-specific therapeutic complex of claim 112 wherein said antibody is selected from the group consisting of a polyclonal antibody, a monoclonal antibody, a humanized antibody, an antibody fragment Fab, an antibody fragment Fab′, an antibody fragment F(ab′)₂, and a single chain Fv.

114. The prostate-specific therapeutic complex of claim 109 wherein said prostate specific molecule is Na/K ATPase beta-1 subunit or a homolog thereof.

115. The prostate-specific therapeutic complex of claim 109 wherein said prostate specific molecule is a polypeptide having an amino acid sequence of SEQ ID NO 31, SEQ ID NO 33 or a homolog thereof.

116. The prostate-specific therapeutic complex of claim 109 wherein said linker is selected from the group consisting of a bond, a peptide, a liposome, and a microcapsule.

117. The prostate-specific therapeutic complex of claim 109 wherein said linker is cleavable.

118. The prostate-specific therapeutic complex of claim 117 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

119. The prostate-specific therapeutic complex of claim 109 wherein said linker is non-cleavable.

120. The prostate-specific therapeutic complex of claim 119 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

121. The prostate-specific therapeutic complex of claim 109 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

122. The prostate-specific therapeutic complex of claim 121 wherein said protein is an enzyme.

123. The prostate-specific therapeutic complex of claim 122 wherein said enzyme cleaves a prodrug.

124. The prostate-specific therapeutic complex of claim 109 wherein said therapeutic moiety is cisplatin alone or in combination with one or more other agents.

125. A pharmaceutical composition comprising a prostate specific therapeutic complex of claim 109 and a pharmaceutically acceptable carrier.

126. A method of treating a patient having a prostate condition comprising administering to said patient a therapeutically effective amount of a colon-specific therapeutic complex wherein said therapeutic complex comprises a ligand capable of selectively binding to lung tissue, a therapeutic moiety, and a linker that links said ligand to said therapeutic moiety.

127. The method of claim 126 wherein said ligand is capable of selectively binding to a lumen exposed molecule on said prostate tissue.

128. The method of claim 127 wherein said lumen exposed molecule is a polypeptide.

129. The method of claim 126 wherein said ligand is capable of selectively binding to a CD73.

130. The method of claim 126 wherein said ligand is capable of selectively binding to a polypeptide having an amino acid sequence of SEQ ID NO 15 or a homolog thereof.

131. The method of claim 126 wherein said linker is selected from the group consisting of a bond, a peptide, a liposome, and a microcapsule.

132. The method of claim 126 wherein said linker is non-cleavable.

133. The method of claim 132 wherein said non-cleavable linker is selected from the group consisting of sulfosuccinimidyl 6-[alpha-methyl-alpha-(2-pyridylthio)toluamido}hexanoate; azidobenzoyl hydrazide; N-hydroxysuccinimidyl-4-azidosalicyclic acid; sulfosuccinimidyl 2-(p-azidosalicylamido)ethyl-1,3-dithiopropionate; N-(4-[p-azidosalicylamido]butyl)-3′(2′-pyidyldithio)propionamide; bis-[beta-(4-azidosalicylamido)ethyl]disulfide; N-hydroxysuccinimidyl-4 azidobenzoate; p-azidophenyl glyoxal monohydrate; N-succimiidyl-6(4′-azido-2′-mitrophenyl-amimo)hexanoate; sulfosuccinimidyl 6-(4′-azido-2′nitrophenylamino)hexanoate; N-5-azido-2-nitrobenzyoyloxysuccinimide; sulfosuccinimidyl-2-(m-azido-o-mitrobenzamido)-ethyl-1,3′-dithiopropionate; p-nitrophenyl-2-diazo-3,3,3-trifluoropropionate; succinimidyl 4-(n-maleimidomethyl)cyclohexane-1-carboxylate; sulfosuccinimidyl 4-(N-maleimidomethyl)cyclohexane-1-carboxylate; m-maleimidobenzoyl-N-hydroxysuccinimide ester; m-maleimidobenzoyl-N-hydroxysulfosuccinimide ester; N-succinimidyl(4-iodoacetyl)aminobenzoate; N-Sulfosuccinimidyl(4-iodoacetyl)aminobenzoate; succinimidyl 4-(p-malenimidophenyl)butyrate; sulfosuccinimidyl 4-(p-malenimidophenyl)butyrate; disuccinimidyl suberate; bis(sulfosuccinimidyl) suberate; bis maleimidohexane; 1,5-difluoro-2,4-dinitrobenzene; dimethyl adipimidate 2 HCl; dimethyl p-imelimidate-2HCl; dimethyl suberimidate-2-HCl; N-succinimidyl-3-(2-pyridylthio)propionate; sulfosuccinimidyl 4-(p-azidophenyl)butyrate; sulfosuccinimidyl 4-(p-azidophenylbutyrate); 1-p-azidosalicylamido)-4-(iodoacetamido)butane; and 4-(p-azidosalicylamido)butylamine.

134. The method of claim 126 wherein said linker is cleavable.

135. The method of claim 134 wherein said cleavable linker is selected from the group consisting of: a linker cleavable under a reducing condition, a linker cleavable under an acidic condition, a linker cleavable by an enzyme or a chemical, a linker cleavable under a basic condition, and a photocleavable linker.

136. The method of claim 126 wherein said therapeutic moiety is selected from the group consisting of a protein, an antibody, an oligonucleotide, a peptide nucleic acid, a small or large organic or inorganic molecule, a polysaccharide, an immuno-modulator, an immuno-suppressor, an anesthetic, an anti-inflammatory, a vitamin, a blood pressure modulator, a chemotherapeutic agent, an anti-neoplastic agent, an antiviral agent, an antifungal agent, an anti-protozoan, a contrast agent, a steroid, an anticoagulant, a coagulant, a prodrug, a radionucleotide, a chromogenic label, a non-enzymatic label, a catalytic label, a chemiluminescent label, and a toxin.

137. The method of claim 126 wherein said therapeutic moiety is cisplatin alone or in combination with one or more other agents.

138. The method of claim 126 wherein said prostate condition is selected from the group consisting of benign prostatic hyperplasia, prostatatis and prostate cancer.

139. The method of claim 126 wherein said therapeutic complex is administered by means selected from the group consisting of orally, parenterally by inhalation, topically, rectally, ocularly nasally, buccally, vaginally, sublingually, transbuccally, liposomally, via an implanted reservoir, and via local delivery.

140. A method of determining the presence or concentration of Na/K ATPase beta-1 subunit or a homolog thereof in a tissue, organ, or cell comprising administering the therapeutic complex of claim 114 to said tissue, organ, or cell and identifying or quantifying the amount of bound therapeutic complex.
